(19) United States
(12) Patent Application Publication
(10) Pub. No.: US 2023/0212695 A1
(43) Pub. Date: Jul. 6, 2023

(54) SELECTIVE ENRICHMENT BROTH FOR DETECTION OF ONE OR MORE PATHOGENS

(71) Applicant: POLYSKOPE LABS, Oklahoma City, OK (US)

(72) Inventors: Cyrus Cody ZEGRATI, Oklahoma City, OK (US); Michael Benjamin CENTOLA, Oklahoma City, OK (US); Paul Simon SMITH, Oklahoma City, OK (US)

(73) Assignee: POLYSKOPE LABS, Oklahoma City, OK (US)

(21) Appl. No.: 17/440,659

(22) PCT Filed: Mar. 13, 2020
(86) PCT No.: PCT/US2020/022831

§ 371 Date: Sep. 17, 2021

Related U.S. Application Data
(60) Provisional application No. 62/819,417, filed on Mar. 15, 2019.

(51) Int. Cl. C12Q 1/689

(52) U.S. Cl. CPC C12Q 1/689; C12Q 1/6806

(57) ABSTRACT

Provided herein are media, methods, kits, primers and oligonucleotide probes for use in the molecular detection of pathogens. These may be used in combination for the rapid, high-throughput screening PCR-based techniques to simultaneously detect multiple pathogens. The multiplex-detection methods have improved sensitivity and specificity for the detection of multiple pathogens simultaneously. Real-time PCR assaying techniques using such primers include microarrays and multiplex arrays, the latter optionally simultaneously with oligonucleotide TaqMan probes.

### CROSS-REFERENCE

This application claims the benefit of U.S. provisional application 62/819,417 filed on Mar. 15, 2019, which is herein incorporated by reference in its entirety.

### BACKGROUND

Common pathogens in food are a major cause of food poisoning, and can cause a variety of diseases. These diseases are a serious threat to people's health. Rapid and accurate detection of foodborne pathogens are an effective tool to combat such diseases.

With the development of molecular biology, food inspection, quarantine work and pathogen identification methods have used technological developments such as PCR, and oligonucleotide hybridizing probes, but it remains difficult with current detection methods in molecular biology to simultaneously detect the presence and/or absence and or screen one or more (plurality) pathogens. The object of the present invention is to provide methods, kits, and compositions for multiple pathogen detection that can detect contaminating pathogens including but not limited to pathogenic Escherichia coli STEC, Salmonella species, and Listeria species by using multiplex PCR with high sensitivity and repeatability.

### SUMMARY OF THE DISCLOSURE

Provided herein are methods for detecting the presence and/or absence of the one or more pathogens.

In some aspects, disclosed herein are methods for detecting the presence or absence of two or more pathogens in a sample. In some aspects, the method can comprise performing an amplification of a selective enrichment media contacted sample. In some aspects, the method can comprise detecting the presence or absence of the two or more pathogens. In some aspects, the two or more pathogens can comprise Escherichia. In some aspects, the two or more pathogens can comprise Salmonella. In some aspects, the two or more pathogens can comprise Listeria species. In aspects, Listeria species can comprise one or more of L. aquatica, L. booriae, L. cornellensis, L. costaricensis, L. goaensis, L. fleischmannii, L. floridensis, L. grandensis, L. grayi, L. innocua, L. ivanovii, L. marthii, L. newyorkensis, L. riparia, L. rocourtiae, L. seeligeri, L. thailandensis, L. weihenstephanensis, or L. welshimeri. In some aspects, the amplification can be performed with primers pairs. In some aspects, the primer pairs can comprise sequences of at least 15 contiguous bases that are at least 70% homologous with AAGCTCATTTCACATCGTCCATCTT sense, ATCCACCATTCCCAAGCTAAACCT antisense. In some aspects, the primer pairs can comprise sequences of at least 15 contiguous bases that are at least 70% homologous with AGTTGGMTTYGGTCGYGTATAAT sense, ACATMDWGCACCRTCTTTCATYAAGT antisense. In some aspects, the primer pairs can comprise sequences of at least 15 contiguous bases that are at least 70% homologous with AGGCGGCCAGATTCAGCATAGT sense, AGCTACCACCTTGCACATAAGCT antisense. In some aspects, the primer pairs can comprise sequences of at least 15 contiguous bases that are at least 70% homologous with ACAGTGCCCGGTGTGACAACT sense, AGACACGTTGCAGAGTGGTATAACT antisense. In some aspects, the primer pairs can comprise sequences of at least 15 contiguous bases that are at least 70% homologous with ACGGGCATACCATCCAGAGAAT sense, ACACCGTGGTCCAGTTTATCGTT antisense. In some aspects, the primer pairs can comprise sequences of at least 15 contiguous bases that are at least 70% homologous with ATGGCGGGACTATTCTGAATGAGT sense, ACATCTCGCTGCTGTCTTTCTTCT antisense. In some aspects, the primer can pairs comprise sequences of at least 15 contiguous bases that are at least 70% homologous with AATGCAGATAAATCGCCATTCGTTGAT sense, AACATCGCTCTTGCCACAGACTGT antisense. In some aspects, the primer pairs can comprise sequences of at least 15 contiguous bases that are at least 70% homologous with ATCGCCATTCGTTGACTACTTCT sense AACATCGCTCTTGCCACAGACTT antisense. In some aspects, the primer pairs can comprise sequences of at least 15 contiguous bases that are at least 70% homologous with ACGGGCATACCATCCAGAGAAT sense, ACACCGTGGTCCAGTTTATCGTT antisense. In some aspects, the primer pairs can comprise sequences of at least 15 contiguous bases that are at least 70% homologous with ACCCACAAAGCAGAAGCAAAAGT sense, ACAGGAACGCCATATTTGACAGT antisense. In some aspects, the method can be performed within a positive total time of about 28 hrs. In some aspects, the selective enrichment media can comprise, per 1 L of water: between about 0 g/L and about 8.0 g/L beef heart solids; between about 0 g/L and about 10.0 g/L calf brain solids; between about 0 g/L and about 35.0 g/L calf brain-beef heart infusion; between about 0 g/L and about 16.0 g/L casein peptone; between about 0 g/L and about 10.0 g/L dextrose; between about 0 g/L and about 7.0 g/L dipotassium phosphate; between about 0 g/L and about 20.0 g/L disodium phosphate; between about 0 g/L and about 8.0 g/L enzymatic digest of soy; between about 0 g/L and about 3.0 g/L esculin; between about 0 g/L and about 10 g/L ferric ammonium citrate; between about 0 g/L and about 8.0 g/L meat peptone; between about 0 g/L and about 10 g/L sodium chloride; between about 0 g/L and about 35.0 g/L pancreatic digest of casein; between about 0 g/L and about 10.0 g/L peptic digest of animal tissue; between about 0 g/L and about 12 g/L porcine brain heart infusion; between about 0 g/L and about 5.0 g/L potassium phosphate; between about 0 g/L and about 4.0 g/L sodium pyruvate; between about 0 g/L and about 14.0 g/L yeast extract; between about 0 g/L and about 15.0 g/L acriflavine hydrochloride; between about 0 g/L and about 0.3 g/L cycloheximide; between about 0 g/L and about 10.0 g/L lithium chloride; or between about 0 g/L and about 0.1 g/L nalidixic acid. In some aspects, the sample can be suspended in the selective enrichment media such that the two or more pathogens are isolated from the sample. In some aspects, the two or more pathogens can be isolated from the sample by stomaching. In some aspects, the sample can be stomached for at least about 30 seconds. In some aspects, the sample can be incubated for a positive amount of time less than or equal to about 24 hours following stomaching. In some aspects, the sample is lysed by incubating the sample with a lysis buffer. In some aspects, the lysis buffer can comprise a buffering component; In some aspects, the lysis buffer can comprise a metal chelating agent; a surfactant; a precipitant; and/or at least two lysing moieties. In some aspects, the buffering component can comprise tris (hydroxymethyl) aminomethane (TRIS). In some aspects, tris (hydroxymethyl) aminomethane (TRIS) can be present at a concentration in the range of about 60 mM to about 100 mM. In some aspects, the metal chelating agent can comprise ethylenediaminetetraacetic acid (EDTA). In some aspects, ethylenediaminetetraacetic acid (EDTA) can be present at a concentration in the range of about 1 mM to about 18 mM. In some aspects, the surfactant can comprise polyethylene glycol p-(1, 1, 3, 3-tetramethylbutyl)-phenyl ether (Triton-X-100). In some aspects, the polyethylene glycol p-(1, 1, 3, 3-tetramethylbutyl)-phenyl ether (Triton-X-100) can be present at a concentration in the range of about 0.1% to about 10%. In some aspects, the precipitant can comprise proteinase K. In some aspects, proteinase K can be present at a concentration in the range of about 17.5% to about 37.5%. In some aspects, the lysing moiety can comprise a lysis bead. In some aspects, the lysis bead can comprise 100 μm zirconium lysis beads. In some aspects, the 100 μm zirconium lysis beads can be present at a concentration in the range of about 0.1 grams/ml to about 2.88 grams/ml. In some aspects, the lysing moiety can comprise lysozyme. In some aspects, the lysozyme can be present at a concentration in the range of about 10 mg/ml to about 30 mg/ml. In some aspects, the one or more pathogens can comprise Escherichia, Salmonella or Listeria species. In some embodiments, a method disclosed herein can be performed without extracting nucleic acids from the one or more pathogens. In some embodiments, nucleic acids can comprise DNA, RNA or a combination thereof.

In some aspects, disclosed herein are methods for enriching a sample. In some aspects, the method comprises conducting a first sample lysis and a second sample lysis on an enriched sample or a portion thereof. In some aspects, the enriched sample was enriched in a selective enrichment media. In some aspects, the second sample lysis can be performed at a temperature higher than a temperature of the first sample lysis, thereby forming a lysed sample. In some aspects, the method comprises conducting amplification with a set of amplification primers on the lysed sample. In some aspects, the amplification primers comprise one or more primer pairs. In some aspects, a first primer of the one or more primer pairs can hybridize to a target nucleic acid sequence of one or more pathogens. In some aspects, a second primer of the one or more primer pairs can hybridize to a sequence complimentary to the target nucleic acid. In some aspects, the method can comprise detecting a presence or absence of the one or more pathogens. In some aspects, the one or more pathogens can comprise Escherichia, Salmonella, or Listeria species. In aspects, Listeria species can comprise one or more of L. aquatica, L. booriae, L. cornellensis, L. costaricensis, L. goaensis, L. fleischmannii, L. floridensis, L. grandensis, L. grayi, L. innocua, L. ivanovii, L. marthii, L. newyorkensis, L. riparia, L. rocourtiae, L. seeligeri, L. thailandensis, L. weihenstephanensis, or L. welshimeri In some aspects, the amplification can be performed with primers pairs. In some aspects, the primer pairs can comprise sequences of at least 15 contiguous bases that are at least 70% homologous with AAGCTCATTTCACATCGTCCATCTT sense, ATCCACCATTCCCAAGCTAAACCT antisense. In some aspects, the primer pairs can comprise sequences of at least 15 contiguous bases that are at least 70% homologous with AGTTGGMTTYGGTCGYGTATAAT sense, ACATMDWGCACCRTCTTTCATYAAGT antisense. In some aspects, the primer pairs can comprise sequences of at least 15 contiguous bases that are at least 70% homologous with AGGCGGCCAGATTCAGCATAGT sense, AGCTACCACCTTGCACATAAGCT antisense. In some aspects, the primer pairs can comprise sequences of at least 15 contiguous bases that are at least 70% homologous with ACAGTGCCCGGTGTGACAACT sense, AGACACGTTGCAGAGTGGTATAACT antisense. In some aspects, the primer pairs can comprise sequences of at least 15 contiguous bases that are at least 70% homologous with ACGGGCATACCATCCAGAGAAT sense, ACACCGTGGTCCAGTTTATCGTT antisense. In some aspects, the primer pairs can comprise sequences of at least 15 contiguous bases that are at least 70% homologous with ATGGCGGGACTATTCTGAATGAGT sense, ACATCTCGCTGCTGTCTTTCTTCT antisense. In some aspects, the primer can pairs comprise sequences of at least 15 contiguous bases that are at least 70% homologous with AATGCAGATAAATCGCCATTCGTTGAT sense, AACATCGCTCTTGCCACAGACTGT antisense. In some aspects, the primer pairs can comprise sequences of at least 15 contiguous bases that are at least 70% homologous with ATCGCCATTCGTTGACTACTTCT sense AACATCGCTCTTGCCACAGACTT antisense. In some aspects, the primer pairs can comprise sequences of at least 15 contiguous bases that are at least 70% homologous with ACGGGCATACCATCCAGAGAAT sense, ACACCGTGGTCCAGTTTATCGTT antisense. In some aspects, the primer pairs can comprise sequences of at least 15 contiguous bases that are at least 70% homologous with ACCCACAAAGCAGAAGCAAAAGT sense, ACAGGAACGCCATATTTGACAGT antisense. In some aspects, the method can be performed within a positive total time of about 28 hrs. In some aspects, the selective enrichment media can comprise, per 1 L of water: between about 0 g/L and about 8.0 g/L beef heart solids; between about 0 g/L and about 10.0 g/L calf brain solids; between about 0 g/L and about 35.0 g/L calf brain-beef heart infusion; between about 0 g/L and about 16.0 g/L casein peptone; between about 0 g/L and about 10.0 g/L dextrose; between about 0 g/L and about 7.0 g/L dipotassium phosphate; between about 0 g/L and about 20.0 g/L disodium phosphate; between about 0 g/L and about 8.0 g/L enzymatic digest of soy; between about 0 g/L and about 3.0 g/L esculin; between about 0 g/L and about 10 g/L ferric ammonium citrate; between about 0 g/L and about 8.0 g/L meat peptone; between about 0 g/L and about 10 g/L sodium chloride; between about 0 g/L and about 35.0 g/L pancreatic digest of casein; between about 0 g/L and about 10.0 g/L peptic digest of animal tissue; between about 0 g/L and about 12 g/L porcine brain heart infusion; between about 0 g/L and about 5.0 g/L potassium phosphate; between about 0 g/L and about 4.0 g/L sodium pyruvate; between about 0 g/L and about 14.0 g/L yeast extract; between about 0 g/L and about 15.0 g/L acriflavine hydrochloride; between about 0 g/L and about 0.3 g/L cycloheximide; between about 0 g/L and about 10.0 g/L lithium chloride; or between about 0 g/L and about 0.1 g/L nalidixic acid. In some aspects, the two or more pathogens can be isolated from the sample by stomaching. In some aspects, the sample can be stomached for at least about 30 seconds. In some aspects, the sample can be suspended in the selective enrichment media such that the one or more pathogens are isolated from the sample. In some aspects, the one or more pathogens can be isolated from the sample by stomaching. In some aspects, the sample can be stomached for at least about 30 seconds. In some aspects, the sample can be enriched at a temperature in the range of about 30° C. to about 45° C. In some aspects, the sample can be incubated for a positive amount of time less than or equal to about 24 hours following stomaching. In some aspects, the sample can be lysed by incubating the sample with a lysis buffer. In some aspects, the lysis buffer can comprise a buffering component; In some aspects, the lysis buffer can comprise a metal chelating agent; a surfactant; a precipitant; and/or at least two lysing moieties. In some aspects, the buffering component can comprise tris (hydroxymethyl) aminomethane (TRIS). In some aspects, tris (hydroxymethyl) aminomethane (TRIS) can be present at a concentration in the range of about 60 mM to about 100 mM. In some aspects, the metal chelating agent can comprise ethylenediaminetetraacetic acid (EDTA). In some aspects, ethylenediaminetetraacetic acid (EDTA) can be present at a concentration in the range of about 1 mM to about 18 mM. In some aspects, the surfactant can comprise polyethylene glycol p-(1, 1, 3, 3-tetramethylbutyl)-phenyl ether (Triton-X-100). In some aspects, the polyethylene glycol p-(1, 1, 3, 3-tetramethylbutyl)-phenyl ether (Triton-X-100) can be present at a concentration in the range of about 0.1% to about 10%. In some aspects, the precipitant can comprise proteinase K. In some aspects, proteinase K can be present at a concentration in the range of about 17.5% to about 37.5%. In some aspects, the lysing moiety can comprise a lysis bead. In some aspects, the lysis bead can comprise 100 μm zirconium lysis beads. In some aspects, the 100 μm zirconium lysis beads can be present at a concentration in the range of about 0.1 grams/ml to about 2.88 grams/ml. In some aspects, the lysing moiety can comprise lysozyme. In some aspects, the lysozyme can be present at a concentration in the range of about 10 mg/ml to about 30 mg/ml. In some aspects, the method can comprise hybridization of an internal oligonucleotide probe to a sequence within a target sequence or a complement thereof. In some aspects, the internal oligonucleotide probe does not hybridize to the amplification primers. In some aspects, the hybridization of the internal oligonucleotide probe to a sequence within the target sequence or a complement thereof can be indicative of the presence of the one or more pathogen in the sample. In some aspects, the internal oligonucleotide probe can be labeled at its 5′end with an energy transfer donor fluorophore and labeled at its 3′end with an energy transfer acceptor fluorophore. In some aspects, the detecting can be reported by a communication medium. In some aspects, the one or more pathogens can comprise Escherichia, Salmonella and or Listeria species. In some embodiments, a method disclosed herein can be performed without extracting nucleic acids from the one or more pathogens. In some embodiments, nucleic acids can comprise DNA, RNA or a combination thereof.

Disclosed herein are compositions. In some aspects, the composition can be configured after contacting at least two pathogens to grow the at least two pathogens. In some aspects, the least two pathogens can comprise Escherichia. In some aspects, the least two pathogens can comprise Salmonella. In some aspects, the least two pathogens can comprise Listeria species. In aspects, Listeria species can comprise one or more of L. aquatica, L. booriae, L. cornellensis, L. costaricensis, L. goaensis, L. fleischmannii, L. floridensis, L. grandensis, L. grayi, L. innocua, L. ivanovii, L. marthii, L. newyorkensis, L. riparia, L. rocourtiae, L. seeligeri, L. thailandensis, L. weihenstephanensis, or L. welshimeri. In some aspects, the at least two pathogens can comprise Escherichia, Salmonella and Listeria species. In some aspects, the composition can comprise, per 1 L of water: between about 0 g/L and about 8.0 g/L beef heart solids; between about 0 g/L and about 10.0 g/L calf brain solids; between about 0 g/L and about 35.0 g/L calf brain-beef heart infusion; between about 0 g/L and about 16.0 g/L casein peptone; between about 0 g/L and about 10.0 g/L dextrose; between about 0 g/L and about 7.0 g/L dipotassium phosphate; between about 0 g/L and about 20.0 g/L disodium phosphate; between about 0 g/L and about 8.0 g/L enzymatic digest of soy; between about 0 g/L and about 3.0 g/L esculin; between about 0 g/L and about 10 g/L ferric ammonium citrate; between about 0 g/L and about 8.0 g/L meat peptone; between about 0 g/L and about 10 g/L sodium chloride; between about 0 g/L and about 35.0 g/L pancreatic digest of casein; between about 0 g/L and about 10.0 g/L peptic digest of animal tissue; between about 0 g/L and about 12 g/L porcine brain heart infusion; between about 0 g/L and about 5.0 g/L potassium phosphate; between about 0 g/L and about 4.0 g/L sodium pyruvate; or between about 0 g/L and about 14.0 g/L yeast extract. In some aspects, the composition can comprise a selective agent. In some aspects, the selective agent can comprise Acriflavine hydrochloride, Cycloheximide, Lithium Chloride or Nalidixic. In some aspects, the selective agent can comprise the Acriflavine hydrochloride, wherein the Acriflavine hydrochloride can be present at 0-0.5 g/L. In some aspects, the selective agent can comprises Cycloheximide, wherein the Cycloheximide can be present at 0-0.8 g/L. In some aspects, the selective agent can comprise the Lithium Chloride, wherein the Lithium Chloride can be present at 0-10 g/L. In some aspects, the selective agent can comprise the Nalidixic, wherein the Nalidixic can be present at 0-0.9 g/L.

In some aspects, the methods of the present invention may achieve these and other objects via the use of primers in combination for the rapid, high-throughput screening PCR-based techniques to simultaneously detect multiple pathogens. The multiplex-detection methods performed in some aspects of the present invention have improved sensitivity and specificity for the detection of multiple pathogens simultaneously.

In some aspects, the methods described herein comprises primers that detect with high specificity and sensitivity to certain pathogens, and thus the primers described herein may be used in reliable detection techniques as described herein to identify pathogens in the human food supply before the pathogens reach the consumer. Various aspects of the present invention utilize amplifiable PCR product sizes, allowing the methods to also be useful in the identification of pathogens and their closely related variants for the purpose of classifying and tracing the origin of contamination.

In some aspects, organisms for which the method can detect include but is not limited to relevant species, subspecies, serovars, and/or strains of for example, Escherichia coli O157:H7, Shigella dysenteriae, Salmonella enterica ssp. enterica (including serovars Typhi, Typhimurium, and Saintpaul) Francisella tularensis ssp. tularensis, Francisella tularensis ssp. novicida, Vibrio cholerae, Vibrio parahaemolyticus, Shigella sonnei, Yersinia pestis, Listeria monocytogenes and Yersinia pseudotuberculosis. In some aspects, the methods described herein identifies PCR conditions that are suitable for the amplification of all pathogens under the same reaction conditions, thus making the primers thus identified suitable for combined use under those reaction conditions in multiple simultaneous PCR to detect and identify those food pathogens.

In some aspects, sets of multiplex PCR primers and TaqMan probes may be designed using commercial software and genomic DNA sequences. In some aspects, specificity of resulting sequences may be assessed in silico against the nr database using Blast. In some aspects, optimal PCR conditions may be identified for each of the multiplex sets. In some aspects, selection of a final set of primers and probes may be done in a step-wise manner. In some aspects, compatibility, sensitivity, and specificity may be assessed using purified genomic DNA from target organisms and with non-target bacteria DNA. In some aspects, sets of primers and probes with optimal performance in with non-target bacteria DNA may then be further tested using DNA prepared from cultured bacteria. In some aspects, sets of primers and probes may be tested using DNA prepared from bacteria cultured in the presence of various food matrices.

In some aspects, the methods described herein can identify primers for pathogens that may be readily combined into common assays for the rapid and accurate detection of pathogens, wherein the assays are capable of discriminating a broad range of pathogens or related bacteria.

In some aspects, the methods described herein can identify various primers may be used alone to detect and identify a selected pathogen, or may be used in combination and/or tandem to detect and identify whether any of a plurality of pathogens are present in a sample.

In some aspects, when used in tandem or combination, the primers and or oligonucleotide probes described herein may comprise using primer pairs or oligonucleotide probes designed for detecting two or more different pathogens in a common PCR-microplate array or, alternatively, in a multiplex PCR. In some aspects, the various different primer pairs and or oligonucleotide probes are selected such that all utilized pairs can operate under the same conditions (e.g., melting temperatures) such that the PCR process can be run in simultaneously on the microarray or one-tube array, or together in an assay. In some aspects, the microarrays and/or multiplex arrays contain primer pairs and or oligonucleotide probes sufficient to detect and identify two, three, four, five, six or more pathogens simultaneously. In some aspects, particularly with respect to multiplex PCR, such embodiments can optionally use different probes specific to the target gene containing different dyes of different emission capacity to assist in multiplex detection.

Further disclosed herein include system and device for detecting one or more pathogens. The device and system can be a computer system. The device and system can comprise a memory that stores executable instructions and a processor to execute the executable instructions to perform any methods for detecting one or more pathogens. In some cases, the device and system can detect one or more pathogens in a sample using the oligonucleotide probes, primers, and lysis buffers in the kits disclosed herein. In some aspects, the device and system can detect the presence or absence of one or more pathogens.

### INCORPORATION BY REFERENCE

All publications, patents, and patent applications mentioned in this specification are herein incorporated by reference in their entirety for all purposes, to the same extent as if each individual publication, patent, or patent application is specifically and individually indicated to be incorporated by reference in their entireties.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 depicts a graph showing 1 CFU/25 g—Fresh Spinach—STX-1 and STX-2 Targets

FIG. 2 depicts a graph showing 1 CFU/25 g—Fresh Spinach—E. coli EAE Targets

FIG. 3 depicts a graph showing 1 CFU/25 g—Fresh Spinach—L. monocytogenes Target

FIG. 4 depicts a graph showing 1 CFU/25 g—Fresh Spinach—Salmonella enterica Target

FIG. 5 depicts a graph showing 1 CFU/25 g—Fresh Spinach—All Targets

FIG. 6 depicts a chart showing 1 CFU/25 g—Fresh Spinach—Table of Results

FIG. 7 depicts a graph showing 5 CFU/25 g—Fresh Spinach—STX-1 and STX-2 Targets

FIG. 8 depicts a graph showing 5 CFU/25 g—Fresh Spinach—E. coli EAE Targets

FIG. 9 depicts a graph showing 5 CFU/25 g—Fresh Spinach—L. monocytogenes Target

FIG. 10 depicts a graph showing 5 CFU/25 g—Fresh Spinach—Salmonella enterica Target

FIG. 11 depicts a graph showing 5 CFU/25 g—Fresh Spinach—All Targets

FIG. 12 depicts a chart showing 5 CFU/25 g—Fresh Spinach—Table of Results

FIG. 13 depicts a graph showing 1 CFU/25 g—Raw Ground Beef—STX-1 and STX-2 Targets

FIG. 14 depicts a graph showing 1 CFU/25 g—Raw Ground Beef—E. coli EAE Targets

FIG. 15 depicts a graph showing 1 CFU/25 g—Raw Ground Beef—L. monocytogenes Target

FIG. 16 depicts a graph showing 1 CFU/25 g—Raw Ground Beef—Salmonella enterica Target

FIG. 17 depicts a graph showing 1 CFU/25 g—Raw Ground Beef—All Targets

FIG. 18 depicts a chart showing 1 CFU/25 g—Raw Ground Beef—Table of Results

FIG. 19 depicts a graph showing 5 CFU/25 g—Raw Ground Beef—STX-1 and STX-2 Targets

FIG. 20 depicts a graph showing 5 CFU/25 g—Raw Ground Beef—E. coli EAE

FIG. 21 depicts a graph showing 5 CFU/25 g—Raw Ground Beef—L. monocytogenes Target

FIG. 22 depicts a graph showing 5 CFU/25 g—Raw Ground Beef—Salmonella enterica Target

FIG. 23 depicts a graph showing 5 CFU/25 g—Raw Ground Beef—All Targets

FIG. 24 depicts a chart showing 5 CFU/25 g—Raw Ground Beef—Table of Results

FIG. 25 depicts a graph showing 15 CFU/25 g—Milk—All Listeria Targets

FIG. 26 depicts a graph showing 15 CFU/25 g—Milk—All Listeria Targets

FIG. 27 depicts a graph showing 2 CFU/25 g—Milk—Listeria ivanovii Target

FIG. 28 depicts a graph showing 2 CFU/25 g—Milk—Listeria ivanovii Target

FIG. 29 depicts a graph showing 2 CFU/25 g—Milk—Listeria monocytogenes Target

FIG. 30 depicts a graph showing 2 CFU/25 g—Milk—Listeria monocytogenes Target

FIG. 31 depicts a graph showing 2 CFU/25 g—Milk—Listeria seeligeri Target

FIG. 32 depicts a graph showing 2 CFU/25 g—Milk—Listeria seeligeri Target

FIG. 33 depicts a graph showing 2 CFU/25 g—Milk—Listeria welshimeri Target

FIG. 34 depicts a graph showing 2 CFU/25 g—Milk—Listeria welshimeri Target

FIG. 35 depicts a graph showing 2 CFU/25 g—Cheddar—Listeria ivanovii Target

FIG. 36 depicts a graph showing 2 CFU/25 g—Cheddar—Listeria ivanovii Target

FIG. 37 depicts a graph showing 2 CFU/25 g—Cheddar—S. enterica Target

FIG. 38 depicts a graph showing 2 CFU/25 g—Cheddar—E. coli EAE Target

FIG. 39 depicts a graph showing 2 CFU/25 g—Cheddar—L. welshimeri Target

FIG. 40 depicts a graph showing 2 CFU/25 g—Cheddar—L. welshimeri Target

FIG. 41 depicts a graph showing 2 CFU/25 g—Cheddar—L. welshimeri Target

FIG. 42 depicts a graph showing 2 CFU/25 g—Cheddar—L. welshimeri Target

FIG. 43 depicts a graph showing 2 CFU/25 g—Cheddar—L. monocytogenes Target

FIG. 44 depicts a graph showing 2 CFU/25 g—Cheddar—L. monocytogenes Target

FIG. 45 depicts a graph showing 2 CFU/25 g—Cheddar—L. monocytogenes Target

FIG. 46 depicts a graph showing 2 CFU/25 g—Cheddar—L. ivanovii Target

FIG. 47 depicts a graph showing 2 CFU/25 g—Ricotta—L. ivanovii Target

FIG. 48 depicts a graph showing 2 CFU/25 g—Ricotta—L. ivanovii Target

FIG. 49 depicts a graph showing 2 CFU/25 g—Ricotta—L. ivanovii Target

FIG. 50 depicts a graph showing 2 CFU/25 g—Ricotta—L. ivanovii Target

FIG. 51 depicts a graph showing 2 CFU/25 g—Ricotta—L. welshimeri Target

FIG. 52 depicts a graph showing 2 CFU/25 g—Ricotta—L. welshimeri Target

FIG. 53 depicts a graph showing 2 CFU/25 g—Ricotta—L. welshimeri Target

FIG. 54 depicts a graph showing 2 CFU/25 g—Ricotta—L. welshimeri Target

FIG. 55 depicts a graph showing 2 CFU/25 g—Ricotta—L. monocytogenes Target

FIG. 56 depicts a graph showing 2 CFU/25 g—Ricotta—L. monocytogenes Target

FIG. 57 depicts a graph showing 2 CFU/25 g—Ricotta—L. monocytogenes Target

FIG. 58 depicts a graph showing 2 CFU/25 g—Ricotta—L. monocytogenes Target

FIG. 59 depicts a graph showing 2 CFU/25 g—Deli Turkey—L. innocua Target

FIG. 60 depicts a graph showing 2 CFU/25 g—Ricotta—L. innocua Target

FIG. 61 depicts a graph showing 2 CFU/25 g—Deli Turkey—L. welshimeri Target

FIG. 62 depicts a graph showing 2 CFU/25 g—Ricotta—L. welshimeri Target

FIG. 63 depicts a chart showing 2 CFU/25 g—Ricotta and Deli Turkey—Table of Results

FIG. 64 depicts a graph showing 1 CFU/25 g—Deli Turkey—E. Coli STX-1 and STX-2 Target

FIG. 65 depicts a graph showing 1 CFU/25 g—Deli Turkey—E. Coli STX-1 and STX-2 Target

FIG. 66 depicts a graph showing 1 CFU/25 g—Deli Turkey—E. Coli STX-1 and STX-2

FIG. 67 depicts a graph showing 1 CFU/25 g—Deli Turkey—E. Coli STEC EAE Target

FIG. 68 depicts a graph showing 1 CFU/25 g—Deli Turkey—S. enterica Target

FIG. 69 depicts a graph showing 1 CFU/25 g—Deli Turkey—S. enterica Target

FIG. 70 depicts a graph showing 1 CFU/25 g—Deli Turkey—Listeria spp. Target

FIG. 71 depicts a graph showing 1 CFU/25 g—Deli Turkey—Listeria spp. Target

FIG. 72 depicts a graph showing 1 CFU/25 g—Deli Turkey—All Targets

FIG. 73 depicts a graph showing 1 CFU/25 g—Deli Turkey—All Targets

FIG. 74 depicts a graph showing 5 CFU/25 g—Deli Turkey—E. Coli STX-1 and STX-2 Target

FIG. 75 depicts a graph showing 5 CFU/25 g—Deli Turkey—E. Coli STX-1 and STX-2 Target

FIG. 76 depicts a graph showing 5 CFU/25 g—Deli Turkey—E. Coli STEC EAE Target

FIG. 77 depicts a graph showing 5 CFU/25 g—Deli Turkey—E. Coli STEC EAE Target

FIG. 78 depicts a graph showing 5 CFU/25 g—Deli Turkey—S. enterica Target

FIG. 79 depicts a graph showing 5 CFU/25 g—Deli Turkey—S. enterica Target

FIG. 80 depicts a graph showing 5 CFU/25 g—Deli Turkey—Listeria spp. Target

FIG. 81 depicts a graph showing 5 CFU/25 g—Deli Turkey—Listeria spp. Target

FIG. 82 depicts a graph showing 5 CFU/25 g—Deli Turkey—All Targets

FIG. 83 depicts a graph showing 5 CFU/25 g—Deli Turkey—All Targets

FIG. 84 depicts a graph showing 2 CFU/25 g—Hemp—E. Coli STX-1 and STX-2 Target

FIG. 85 depicts a graph showing 2 CFU/25 g—Hemp—E. Coli STX-1 and STX-2 Target

FIG. 86 depicts a graph showing 2 CFU/25 g—Hemp—E. Coli STEC EAE Target

FIG. 87 depicts a graph showing 2 CFU/25 g—Hemp—E. Coli STEC EAE Target

FIG. 88 depicts a graph showing 2 CFU/25 g—Hemp—S. enterica Target

FIG. 89 depicts a graph showing 2 CFU/25 g—Hemp—S. enterica Target

FIG. 90 depicts a graph showing 2 CFU/25 g—Hemp—All Targets

FIG. 91 depicts a graph showing 2 CFU/25 g—Hemp—All Targets

FIG. 92 depicts a graph showing 15 CFU/25 g—Hemp—E. Coli STX-1 and STX-2 Target

FIG. 93 depicts a graph showing 15 CFU/25 g—Hemp—E. Coli STX-1 and STX-2 Target

FIG. 94 depicts a graph showing 15 CFU/25 g—Hemp—E. Coli STEC EAE Target

FIG. 95 depicts a graph showing 15 CFU/25 g—Hemp—E. Coli STEC EAE Target

FIG. 96 depicts a graph showing 15 CFU/25 g—Hemp—S. enterica Target

FIG. 97 depicts a graph showing 15 CFU/25 g—Hemp—S. enterica Target

FIG. 98 depicts a graph showing 15 CFU/25 g—Hemp—All Targets

FIG. 99 depicts a graph showing 15 CFU/25 g—Hemp—All Targets

FIG. 100 depicts a graph showing 15 CFU/25 g—Hemp—Table of Results

FIG. 101 depicts a graph showing 1 CFU/25 g—Sponge—L. grayi ATCC 19120

FIG. 102 depicts a graph showing 1 CFU/25 g—Sponge—L. grayi ATCC 19120

FIG. 103 depicts a graph showing 1 CFU/25 g—Sponge—L. ivanovii ATCC 19119

FIG. 104 depicts a graph showing 1 CFU/25 g—Sponge—L. ivanovii ATCC 19119

FIG. 105 depicts a graph showing 1 CFU/25 g—Sponge—L. ivanovii ATCC 700402

FIG. 106 depicts a graph showing 1 CFU/25 g—Sponge—L. ivanovii ATCC 700402

FIG. 107 depicts a graph showing 1 CFU/25 g—Sponge—L. innocua ATCC 33090

FIG. 108 depicts a graph showing 1 CFU/25 g—Sponge—L. innocua ATCC 33090

FIG. 109 depicts a graph showing 1 CFU/25 g—Sponge—L. marthii BPBAA 1595

FIG. 110 depicts a graph showing 1 CFU/25 g—Sponge—L. marthii BPBAA 1595

FIG. 111 depicts a graph showing 1 CFU/25 g—Sponge—L. seeligeri ATCC 35967

FIG. 112 depicts a graph showing 1 CFU/25 g—Sponge—L. seeligeri ATCC 35967

FIG. 113 depicts a graph showing 1 CFU/25 g—Sponge—L. welshimeri ATCC 35897

FIG. 114 depicts a graph showing 1 CFU/25 g—Sponge—L. welshimeri ATCC 35897

FIG. 115 depicts a graph showing 1 CFU/25 g—Sponge—Listeria spp. on ABI 7500

FIG. 116 depicts a chart showing a table summarizing the results for Liquid handling robot and Technician run for samples of 1 CFU/25 g Pork Sausage on QuantStudio 5 and ABI 7500 Fast

FIG. 117 depicts a chart showing results for Liquid handling robot Validation.

FIG. 118 depicts a graph showing Liquid handling robot Validation—1 CFU Pork Sausage E. coli O157:H7 ABI 7500 Fast

FIG. 119 depicts a graph showing Liquid handling robot Validation—1 CFU Pork Sausage E. coli O157:H7 ABI 7500 Fast

FIG. 120 depicts a graph showing Liquid handling robot Validation—1 CFU Pork Sausage L. innocua target ABI 7500 Fast.

FIG. 121 depicts a graph showing Liquid handling robot Validation—1 CFU Pork Sausage S. enterica target ABI 7500 Fast.

FIG. 122 depicts a graph showing Liquid handling robot Validation—1 CFU Pork Sausage All Targets Present Quantstudio ABI 7500 Fast

FIG. 123 depicts a table of Results for Liquid handling robot and Technician Run Samples.

FIG. 124 depicts a table of results for Liquid handling robot Validation.

FIG. 125 depicts a graph showing results for Liquid handling robot Validation—5 CFU Pork Sausage E. coli O157:H7 on ABI 7500 Fast.

FIG. 126 depicts a graph showing Liquid handling robot Validation—5 CFU Pork Sausage E. coli O157:H7 ABI 7500 Fast.

FIG. 127 depicts a graph showing Liquid handling robot Validation—5 CFU Pork Sausage L. innocua target ABI 7500 Fast.

FIG. 128 depicts a graph showing Liquid handling robot Validation—5 CFU Pork Sausage S. enterica target ABI 7500 Fast.

FIG. 129 depicts a graph showing Liquid handling robot Validation—5 CFU Pork Sausage All Targets Present Quantstudio ABI 7500 Fast.

FIG. 130 depicts a graph showing Results for Liquid handling robot and Technician Run Samples 1 CFU/sponge on ABI 7500 Fast 5 CFU/25 g Pork Sausage on ABI 7500 Fast.

FIG. 131 depicts a chart showing a table of results for Liquid handling robot Validation.

FIG. 132 depicts a graph showing Liquid handling robot Validation—1 CFU Sponge L. innocua target ABI 7500 Fast.

FIG. 133 depicts a graph showing Liquid handling robot Validation—1 CFU Sponge S. enterica target.

FIG. 134 depicts a graph showing Liquid handling robot Validation—1 CFU Sponge All Targets Present Quantstudio ABI 7500 Fast.

FIG. 135 depicts a Table showing Results for Liquid handling robot and Technician Run Samples 5 CFU/sponge on ABI 7500 Fast.

FIG. 136 depicts a table of results for Liquid handling robot Validation.

FIG. 137 depicts a graph showing Liquid handling robot Validation—5 CFU Sponge L. innocua target ABI 7500 Fast. The Liquid handling robot and technician-run samples both detected the Listeria spp. target in 3/3 replicates (100% recovery).

FIG. 138 depicts a graph showing Liquid handling robot Validation—5 CFU Sponge S. enterica target ABI 7500 Fast. The Liquid handling robot and technician-run samples both detected the S. enterica target in 3/3 replicates (100% recovery).

FIG. 139 depicts a graph showing Liquid handling robot Validation—5 CFU Sponge All Targets Present Quantstudio ABI 7500 Fast. All targets were present in a single reaction.

FIG. 140 depicts a table showing Results for Liquid handling robot and Technician Run Samples 1 CFU/25 g Pork Sausage on Quantstudio 5 and ABI 7500 Fast.

FIG. 141 depicts a table of results for the liquid handling robot validation.

FIG. 142 depicts a graph showing Liquid handling robot Validation—1 CFU Pork Sausage E. coli O157:H7 ABI 7500 Fast.

FIG. 143 depicts a graph showing Liquid handling robot Validation—1 CFU Pork Sausage E. coli O157:H7 ABI 7500 Fast.

FIG. 144 depicts a graph showing Liquid handling robot Validation—1 CFU Pork Sausage L. innocua target ABI 7500 Fast.

FIG. 145 depicts a graph showing Liquid handling robot Validation—1 CFU Pork Sausage S. enterica target ABI 7500 Fast.

FIG. 146 depicts a graph showing Liquid handling robot Validation—1 CFU Pork Sausage All Targets Present Quantstudio ABI 7500 Fast.

FIG. 147 depicts a table showing results for Liquid handling robot and Technician Run Samples 5 CFU/25 g Pork Sausage on ABI 7500 Fast.

FIG. 148 depicts a table showing Liquid handling robot Validation Table of Results.

FIG. 149 depicts a graph showing Liquid handling robot Validation—5 CFU Pork Sausage E. coli O157:H7 ABI 7500 Fast.

FIG. 150 depicts a graph showing Liquid handling robot Validation—5 CFU Pork Sausage E. coli O157:H7 ABI 7500 Fast.

FIG. 151 depicts a graph showing Liquid handling robot Validation—5 CFU Pork Sausage L. innocua target ABI 7500 Fast.

FIG. 152 depicts a graph showing Liquid handling robot Validation—5 CFU Pork Sausage S. enterica target ABI 7500 Fast.

FIG. 153 depicts a graph showing Liquid handling robot Validation—5 CFU Pork Sausage All Targets Present Quantstudio ABI 7500 Fast.

FIG. 154 depicts a table showing Results for Liquid handling robot and Technician Run Samples 1 CFU/sponge on ABI 7500 Fast.

FIG. 155 depicts a table showing a table of results for Liquid handling robot Validation.

FIG. 156 depicts a graph showing Liquid handling robot Validation—1 CFU Sponge L. innocua target ABI 7500 Fast.

FIG. 157 depicts a graph showing Liquid handling robot Validation—1 CFU Sponge S. enterica target.

FIG. 158 depicts a graph showing Liquid handling robot Validation—1 CFU Sponge All Targets Present Quantstudio ABI 7500 Fast.

FIG. 159 depicts a table showing Results for Liquid handling robot and Technician Run Samples 5 CFU/sponge on ABI 7500 Fast.

FIG. 160 depicts a table showing a table of results for Liquid handling robot Validation.

FIG. 161 depicts a graph showing Liquid handling robot Validation—5 CFU Sponge L. innocua target ABI 7500 Fast.

FIG. 162 depicts a graph showing Liquid handling robot Validation—5 CFU Sponge S. enterica target ABI 7500 Fast.

FIG. 163 depicts a graph showing Liquid handling robot Validation—5 CFU Sponge All Targets Present Quantstudio ABI 7500 Fast.

FIG. 164 depicts a table showing PCR and Method Comparison Results 1 CFU/sponge.

FIG. 165 depicts a table showing Sponges—1 CFU Quantstudio 5 and ABI 7500 Fast at 18 Hours.

FIG. 166 depicts a table showing Sponges—1 CFU Quantstudio 5 and ABI 7500 Fast at 24 Hours.

FIG. 167 depicts a table showing Sponges—1 CFU AOAC BAMIMLG Method Comparison Results at 18 and 24 Hours.

FIG. 168 depicts a table showing a table of results for AOAC Method Comparison.

FIG. 169 depicts a graph showing AOAC Method Comparison Validation—1 CFU L. innocua target Quantstudio 5 at 18 Hours.

FIG. 170 depicts a graph showing AOAC Method Comparison Validation—1 CFU L. innocua target ABI 7500 Fast at 18 Hours.

FIG. 171 depicts a graph showing AOAC Method Comparison Validation—1 CFU S. enterica target Quantstudio 5 at 18 Hours.

FIG. 172 depicts a graph showing AOAC Method Comparison Validation—1 CFU S. enterica target ABI 7500 Fast at 18 Hours.

FIG. 173 depicts a graph showing AOAC Method Comparison Validation—1 CFU Both Targets present on Quantstudio 5 at 18 Hours.

FIG. 174 depicts a graph showing AOAC Method Comparison Validation—1 CFU Both Targets present on ABI 7500 Fast at 18 Hours.

FIG. 175 depicts a graph showing AOAC Method Comparison Validation—1 CFU L. innocua target Quantstudio 5 at 24 Hours.

FIG. 176 depicts a graph showing AOAC Method Comparison Validation—1 CFU L. innocua target ABI 7500 Fast at 24 Hours.

FIG. 177 depicts a graph showing AOAC Method Comparison Validation—1 CFU S. enterica target Quantstudio 5 at 24 Hours.

FIG. 178 depicts a graph showing AOAC Method Comparison Validation—1 CFU S. enterica target ABI 7500 Fast at 24 Hours.

FIG. 179 depicts a graph showing AOAC Method Comparison Validation—1 CFU Both Targets present on Quantstudio 5 at 24 Hours.

FIG. 180 depicts a table showing AOAC Method Comparison Validation—1 CFU Both Targets present on ABI 7500 Fast at 24 Hours.

FIG. 181 depicts a table showing PCR and Method Comparison Results* 5 CFU/sponge.

FIG. 182 depicts a table showing Environmental Sponge 5 CFU—QuantStudio 5 and ABI 7500 Fast Results at 18 Hours.

FIG. 183 depicts a table showing Environmental Sponge 5 CFU—QuantStudio 5 and ABI 7500 Fast Results at 24 Hours.

FIG. 184 depicts a table showing Sponges—5 CFU AOAC BAM/MLG Method Comparison Results at 18 and 24 Hours.

FIG. 185 depicts a table showing a table of results for AOAC Method Comparison.

FIG. 186 depicts a graph showing AOAC Method Comparison Validation—5 CFU L. innocua target Quantstudio 5 at 18 Hours.

FIG. 187 depicts a graph showing AOAC Method Comparison Validation—5 CFU L. innocua target ABI 7500 Fast at 18 Hours.

FIG. 188 depicts a graph showing AOAC Method Comparison Validation—5 CFU S. enterica target Quantstudio 5 at 18 Hours.

FIG. 189 depicts a graph showing AOAC Method Comparison Validation—5 CFU S. enterica target ABI 7500 Fast at 18 Hours.

FIG. 190 depicts a graph showing AOAC Method Comparison Validation—5 CFU Both Targets present on Quantstudio 5 at 18 Hours.

FIG. 191 depicts a graph showing AOAC Method Comparison Validation—5 CFU Both Targets present on ABI 7500 Fast at 18 Hours.

FIG. 192 depicts a graph showing AOAC Method Comparison Validation—5 CFU L. innocua target Quantstudio 5 at 24 Hours.

FIG. 193 depicts a graph showing AOAC Method Comparison Validation—5 CFU L. innocua target ABI 7500 Fast at 24 Hours.

FIG. 194 depicts a graph showing AOAC Method Comparison Validation—5 CFU S. enterica target Quantstudio 5 at 24 Hours.

FIG. 195 depicts a graph showing AOAC Method Comparison Validation—5 CFU S. enterica target ABI 7500 Fast at 24 Hours.

FIG. 196 depicts a graph showing AOAC Method Comparison Validation—5 CFU Both Targets present on Quantstudio 5 at 24 Hours.

FIG. 197 depicts a graph showing AOAC Method Comparison Validation—5 CFU Both Targets present on ABI 7500 Fast at 24 Hours.

FIG. 198 depicts a graph showing QuantStudio5, Hemp—2 CFU E. coli O157:H7 STEC STX-1 and STX-2.

FIG. 199 depicts a graph showing QuantStudio5, Hemp—2 CFU E. coli O157:H7 STEC EAE target.

FIG. 200 depicts a graph showing ABI QuantStudio5 Hemp—2 CFU S. enterica target.

FIG. 201 depicts a graph showing QuantStudio5 All Targets in a Single Reaction—2 CFU.

FIG. 202 depicts a graph showing QuantStudio5, Hemp—15 CFU E. coli O157:H7 STEC STX-1 and STX-2.

FIG. 203 depicts a graph showing QuantStudio5, Hemp—15 CFU E. coli O157:H7 STEC EAE target.

FIG. 204 depicts a graph showing QuantStudio5, Hemp—15 CFU S. enterica target.

FIG. 205 depicts a graph showing QuantStudio5, Hemp 15 CFU All Targets Present.

FIG. 206 depicts a table showing Results for Liquid handling robot and Technician Run Samples 1 CFU/25 g Pork Sausage on QuantStudio 5 and ABI 7500 Fast.

FIG. 207 depicts a table showing a table of results for Liquid handling robot Validation.

FIG. 208 depicts a table showing Results for Liquid handling robot and Technician Run Samples 1 CFU/25 g Pork Sausage on QuantStudio 5 and ABI 7500 Fast.

FIG. 209 depicts a graph showing Liquid handling robot Validation—1 CFU Pork Sausage E. coli O157:H7 Quantstudio 5.

FIG. 210 depicts a graph showing Liquid handling robot Validation—1 CFU Pork Sausage E. coli O157:H7 Quantstudio 5.

FIG. 211 depicts a graph showing Liquid handling robot Validation—1 CFU Pork Sausage L. innocua target Quantstudio 5.

FIG. 212 depicts a graph showing Liquid handling robot Validation—1 CFU Pork Sausage S. enterica target Quantstudio 5.

FIG. 213 depicts a graph showing Liquid handling robot Validation—1 CFU Pork Sausage All Targets Present Quantstudio 5.

FIG. 214 depicts a table showing Results for Liquid handling robot and Technician Run Samples 5 CFU/25 g Pork Sausage.

FIG. 215 depicts a table showing Liquid handling robot Validation—Table of Results.

FIG. 216 depicts a graph showing Liquid handling robot Validation—5 CFU Pork Sausage E. coli O157:H7 Quantstudio 5.

FIG. 217 depicts a graph showing Liquid handling robot Validation—5 CFU Pork Sausage E. coli O157:H7 Quantstudio 5.

FIG. 218 depicts a graph showing Liquid handling robot Validation—5 CFU Pork Sausage L. innocua target Quantstudio 5.

FIG. 219 depicts a graph showing Liquid handling robot Validation—5 CFU Pork Sausage S. enterica target Quantstudio 5.

FIG. 220 depicts a graph showing Liquid handling robot Validation—5 CFU Pork Sausage All Targets Present Quantstudio 5.

FIG. 221 depicts a table showing Results for Liquid handling robot and Technician Run Samples 1 CFU/sponge on QuantStudio 5.

FIG. 222 depicts a table showing a table of results for Liquid handling robot Validation.

FIG. 223 depicts a graph showing Liquid handling robot Validation—1 CFU Sponge L. innocua target Quantstudio 5.

FIG. 224 depicts a graph showing Liquid handling robot Validation—1 CFU Sponge S. enterica target.

FIG. 225 depicts a graph showing Liquid handling robot Validation—1 CFU Sponge All Targets Present Quantstudio 5.

FIG. 226 depicts a table showing Results for Liquid handling robot and Technician Run Samples 5 CFU/sponge on QuantStudio 5.

FIG. 227 depicts a table showing a table of results for Liquid handling robot Validation.

FIG. 228 depicts a graph showing Liquid handling robot Validation—5 CFU Sponge L. innocua target Quantstudio 5.

FIG. 229 depicts a graph showing Liquid handling robot Validation—5 CFU Sponge S. enterica target Quantstudio 5.

FIG. 230 depicts a graph showing Liquid handling robot Validation—5 CFU Sponge All Targets Present Quantstudio 5.

### DETAILED DESCRIPTION OF THE DISCLOSURE

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

### Definitions

In this disclosure the term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

In this disclosure the term “media”, “medium”, “broth”, “culture broth” and the like all can refer to a nutrient mixture suitable to culture a desired pathogen which may be a bacteria or microbe strain or species, or virus or infectious agent or biological agent that causes disease or illness to its host.

In this disclosure the term “pathogen” and the like all can refer to bacteria, or microbe strain, or species, or virus, or infectious agents, or biological agents that can cause disease or illness to its host.

In this disclosure the term “microorganism” can be encompassed in the term “pathogen”.

In this disclosure “detecting” a microorganism or pathogen, can mean any process of observing the presence of a pathogen, or a change in the presence of a pathogen, in a sample, whether or not the pathogen or the change in the pathogen is actually detected.

In this disclosure the term “enriched media,” “enrichment media” “rich media” and the like all can refer to media that have been supplemented with highly nutritious materials such as but not limited to blood, serum or yeast extract for the purpose of cultivating fastidious organisms.

In this disclosure “enrichment” of a media can refer to the addition of selected components to promote the growth or other characteristics of one or more desired pathogen. An “enrichment solution” refers to a solution comprising these additional components.

In this disclosure the term “selective agent” can refer to a chemical or culture condition which serves to favor the growth of a desired pathogen or to inhibit the growth of an undesired pathogen.

In this disclosure “selective media” can refer to a media which supports the growth of particular organisms of interest but inhibits the growth of other organisms.

In this disclosure the term “non-selective media” and the like all can refer to media that is substantially free, or free of antibiotics.

In this disclosure the term “selective enrichment supplement” is equivalent to the term “selective agent”.

In this disclosure the term “hybridizing probe” or “internal oligonucleotide probe” can be equivalent to the term “oligonucleotide probe”.

In this disclosure a “supplement” for a culture media can refer to a solution, liquid, solid or other material for addition to a culture medium.

In this disclosure “substantially free” can refer to less than about 10% by weight, or less than about 9% by weight, or less than about 8% by weight, or less than about 7% by weight, or less than about 6% by weight, or less than about 5% by weight, or less than about 4% by weight, or less than about 3% by weight, or less than about 2% by weight, or less than about 1.5% by weight, such as less than about 1% by weight of the ingredient to which it refers.

In this disclosure “amplicon” can refer to the amplified product of a nucleic acid amplification reaction, e.g., the product of amplification of a sequence.

In this disclosure the terms “sample” and “biological sample” can have the same and broadest possible meaning consistent with their context and refer generally and without limitation to anything desired to be tested for the presence of one or more pathogens of interest, and include all such subject matter whether or not it actually contains any pathogens, or any pathogens of interest and whether or not it contains Noroviruses (Norwalk-like viruses), Campylobacter species, Giardia lamblia, Salmonella, Shigella, Cryptosporidium parvum, Clostridium species, Toxoplasma gondii, Staphylococcus aureus, Shiga toxin-producing Escherichia coli (STEC), Yersinia enterocolitica, Bacillus cereus, Bacillus anthracis, Cyclospora cayetanensis, Listeria monocytogenes, Vibrio parahemolyticus V. vulnificus, L. aquatica, L. booriae, L. cornellensis, L. costaricensis, L. goaensis, L. fleischmannii, L. floridensis, L. grandensis, L. grayi, L. innocua, L. ivanovii, L. marthii, L. newyorkensis, L. riparia, L. rocourtiae, L. seeligeri, L. thailandensis, L. weihenstephanensis, or L. welshimeri.

In this disclosure the term “chelating agent” can refer to a “polydentate ligand”. The terms “chelating agent”, “chelator”, “chelant”, and “sequestering agent” are used interchangeably. The chelating agent can be capable of forming multiple bindings to a single atom such as a metal ion, e.g., Mg²⁺ or Ca²⁺.

The term “detergent” as used herein can mean “surfactant”.

In this disclosure the term “beads” can refer to particles, which are of a size in the range of 50 μm to 2 mm, in some aspects 100 μm to 800 μm.

In this disclosure the terms “polynucleotide” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells. In some embodiments, RNA or DNA can be cell-free.

In this disclosure the terms “oligonucleotide” can refer to a relatively short polynucleotide, including, without limitation, single-stranded deoxyribonucleotides, single- or double-stranded ribonucleotides, RNA, DNA hybrids and double-stranded DNAs. Oligonucleotides, such as single-stranded DNA oligonucleotide probes, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

In this disclosure the term “primary label” can refer to a label that can be directly detected, such as a fluorophore.

In this disclosure the “secondary label” can refer to a label that is indirectly detected.

Briefly, and as described in more detail below, disclosed and claimed herein are kits, compositions and methods for determining the presence and/or absence of one or more pathogens in a sample.

Pathogen

In some aspects, the organisms for which the method can detect include but is not limited to relevant species, subspecies, serovars, and/or strains of for example, Escherichia coli O157:H7, Shigella dysenteriae, Salmonella enterica ssp. enterica (including serovars Typhi, Typhimurium, and Saintpaul) Francisella tularensis ssp. tularensis, Francisella tularensis ssp. novicida, Vibrio cholerae, Vibrio parahaemolyticus, Shigella sonnei, Yersinia pestis, Listeria species, L. aquatica, L. booriae, L. cornellensis, L. costaricensis, L. goaensis, L. fleischmannii, L. floridensis, L. grandensis, L. grayi, L. innocua, L. ivanovii, L. marthii, L. newyorkensis, L. riparia, L. rocourtiae, L. seeligeri, L. thailandensis, L. weihenstephanensis, or L. welshimeri, L Monocytogenes and Yersinia pseudotuberculosis.

In some aspects, the invention concerns rapid and accurate methods for detecting foodborne pathogens, including without limitation relevant species, subspecies, serovars, and/or strains of for example, parasites and their eggs, Noroviruses (Norwalk-like viruses), Campylobacter species, Giardia lamblia, Salmonella, Shigella, Cryptosporidium parvum, Clostridium species, Toxoplasma gondii, Staphylococcus aureus, Shiga toxin-producing Escherichia coli (STEC), Yersinia enterocolitica, Bacillus cereus, Bacillus anthracis, Cyclospora cayetanensis, Listeria species, Listeria monocytogenes, Vibrio parahemolyticus and V. vulnificus, Helicobactor, Mycobacterium, Streptococcus, Pseudomonas, Aeromonas hydrophila; Citrobacter freundi, Enterobacter cloacae, Enter o. faecalis, E. coli non-VTEC, Hafnia alvei, Klebsiella pneumoniae, Proteus vulgaris, Pseudomonas aeroginosa.

Pathogenic viruses may be detected in combination with a pathogen detection method as disclosed herein. Examples of pathogenic virus families include, but are not limited to, Adenoviridae, Picornaviridae, Herpesviridae, Hepadnaviridae, Flaviviridae, Retroviridae, Orthomyxoviridae, Paramyxoviridae, Papovaviridae, Rhabdoviridae, and Togaviridae. The term “microorganism” as used in this disclosure includes a virus, bacterium, parasite or parasite's egg.

Time

In some aspects, the one or more pathogens are detected within a positive total time of about 28 hrs or less. In some aspects, the disclosure provides detecting one or more pathogens within a positive total time of about 1 hrs, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, about 21 hrs, about 22 hrs, about 23 hrs, about 24 hrs, about 25 hrs, about 26 hrs, about 27 hrs, about 28 hrs, about 29 hrs, about 30 hrs, about 31 hrs, about 32 hrs, about 33 hrs, about 34 hrs, about 35 hrs, about 36 hrs, about 37 hrs, about 38 hrs, about 39 hrs, about 40 hrs, about 41 hrs, about 42 hrs, about 43 hrs, about 44 hrs, about 45 hrs, about 46 hrs, about 47 hrs, about 48 hrs, about 49-72 hrs, or about 72-96 hrs. In some aspects, the disclosure provides detecting 2 to 10 pathogens. In some embodiments, the disclosure provides detecting 1, 2, 3, 4, 5, 6, 7, 8, 9, 10, 11, 12, 13, 14, 15, 16, 17, 18, 19, or 20 pathogens. In some aspects, the disclosure provides detecting simultaneously 2, 3, 4, 5, 6, 7, 8, 9, 10, 11, 12, 13, 14, 15, 16, 17, 18, 19, 20 pathogens simultaneously. In some aspects, the disclosure provides detecting simultaneously 20 or more pathogens.

Sample

In some embodiments, as will be appreciated by those of skill in the art, the sample may comprise any number of things, including, but not limited to, bodily fluids (including, but not limited to, blood, nasopharyngeal secretions, urine, serum, lymph, saliva, milk, anal and vaginal secretions, and semen) of virtually any organism, with mammalian samples, including livestock, (e.g. sheep, cow, horse, pig, goat, lama, emu, ostrich or donkey), poultry (e.g. chicken, turkey, goose, duck, or game bird), fish (e.g. salmon or sturgeon), laboratory animal (e.g. rabbit, guinea pig, rat or mouse) companion animal (e.g. dog or cat) or a wild animal in captive or free state, environmental samples (including, but not limited to, air, agricultural, water and soil samples); biological warfare agent samples; research samples; purified samples, such as purified genomic DNA, RNA, cell-free nucleci acids, circulating cell-free nucleic acids, proteins, etc.; and raw samples (bacteria, virus, genomic DNA, etc.).

In some embodiments, the sample may be a food, comprising, meats, poultry, fish, seafood, fruits, and vegetables. In some embodiments, the disclosure provides a sample comprising raw food products, cooled or frozen food products, or products that are generally heated prior to consumption. In some embodiments, the sample is not a food product. In some embodiments, the sample may not be a food product In some embodiments, the food products are raw. In some embodiments, the food product could be partially cooked. In some embodiments, the food product could be cooked but may require additional heating prior to consumption. In some embodiments, food products may include meats (beef, pork, lamb, rabbit and/or goat), poultry, wild game (pheasant, partridge, boar and/or bison), fish, vegetables (veggie-patties, veggie hamburgers), combinations of vegetables and meat, egg products (quiches, custards, cheesecakes) and/or baked goods (batters, doughs, cakes, breads, muffins, biscuits, cupcakes, pancakes and the like whether baked, raw or partially baked). In some embodiments, a sample can comprise hemp, CBD oil, cannabis, tetrahydrocannabinol or any derivatives thereof. In some embodiments, a sample can comprise hemp oil, wax, resin, hemp seed food, animal feed, or cloth.

In some embodiments, the sample may be obtained by taking a piece or portion, or by use of a swab, wipe, filter, smear, or any other suitable method, all of which will be readily understood and implemented and selected among by those skilled in the art. In some embodiments, a sample is or comprises food material or is or comprises plant or animal material or is or comprises meat, seafood, fish, vegetables, fruit, salads, premade meals, eggs, dairy produce, combined and uncombined food materials, canned goods, or any other form of fresh, raw, cooked, uncooked, frozen, refrigerated, ground, chopped, canned, heat treated, dried, preserved, refined, or preserved foodstuffs whatsoever. In some embodiments a sample may be taken from an environment, surface, container or location wherein it is desired to determine whether a pathogen of interest is present, for example and without limitation kitchen surfaces, cooking surfaces, food storage containers, eating utensils, refrigerators, freezers, display containers, wrapping materials, live plants and animals and any other environment, location, surface, or material whatsoever that may be of interest to a user. In some embodiments, the sample may be wash solutions of food samples, drinking water, ocean/river water, environment water, mud, or soil. Those skilled in the art will understand and implement suitable methods for selecting, obtaining and handling any sample for use in embodiments. In selected embodiments samples may comprise meat, fish, seafood, vegetables, eggs or dairy produce.

In some embodiments, the sample may be less than or equal to about 25 grams by weight. In some aspects, the sample is about 1 gram, about 2 grams, about 3 grams, about 4 grams, about 5 grams, about 6 grams, about 7 grams, about 8 grams, about 9 grams, about 10 grams, about 11 grams, about 12 grams, about 13 grams, about 14 grams, about 15 grams, about 16 grams, about 17 grams, about 18 grams, about 19 grams, about 20 grams, about 21 grams, about 22 grams, about 23 grams, about 24 grams, or about 25 grams. In some embodiments, the sample may be less than 1 gram.

In some embodiments, the sample may be greater than or equal to about 25 grams by weight. In some embodiments the sample is about 26 grams, about 27 grams, about 28 grams, about 29 grams, about 30 grams, about 31 grams, about 32 grams, about 33 grams, about 34 grams, about 35 grams, about 36 grams, about 37 grams, about 38 grams, about 39 grams, about 40 grams, about 41 grams, about 42 grams, about 43 grams, about 44 grams, about 45 grams, about 46 grams, about 47 grams, about 48 grams, about 49 grams, about 50 grams, about 51 grams, about 52 grams, about 53 grams, about 54 grams, or about 55 grams. In some aspects, the sample is greater than 55 grams by weight.

Enrichment

In some aspects, the sample may be enriched. In some aspects, the sample may be enriched in media. In some aspects, enrichment comprises suspending the sample in the media. In some aspects, the media is a nonselective media, selective media, selective enrichment media, non-selective enrichment media, rich and non-selective media, rich and selective media, or a combination thereof. In some aspects, selective media may contain combinations of selective agents such as antibiotic to inhibit growth of competing microorganisms. In some embodiments, a selective agent can comprise acriflavine hydrochloride, cycloheximide, lithium chloride or nalidiixic acid. In some aspects, selectivity of selective media may be controlled by concentration of the selective agents. In some embodiments, the sample is suspended in a rich and nonselective media. In some embodiments, the sample is suspended in Buffered Listeria Enrichment Broth Base (no supplements). In some aspects, the media may comprise one or more of water, agar, proteins or peptides, growth factors, amino acids, caesein hydrolysate, salts, lipids, carbohydrates, minerals, vitamins, and pH buffers, and may contain extracts such as meat extract, yeast extract, tryptone, phytone, peptone, and malt extract, and may comprise luria bertani (LB) medium. In some aspects, the media may contain extracts such as meat extract, yeast extract, tryptone, phytone, peptone, or malt extract. In some aspects, the media may comprise luria bertani (LB) medium. In some aspects, the media may be simple, complex or defined media and may be enriched media and may be supplemented in a wide variety of ways, all of which will be readily understood by those skilled in the art. In some aspects, the media may comprise MOPS buffer, an Iron (III) salt such as ferric citrate, a magnesium salt such as Magnesium sulphate, a lithium salt such as lithium chloride, and may contain pyruvate. In some embodiments, the media may comprise or consist of any core media as defined herein. In some embodiments, the media may comprise one or more of beef heart solids, calf brain solids, calf brain-beef heart infusion, casein peptone, dextrose, dipotassium phosphate, disodium phosphate, enzymatic digest of soy, esculin, ferric ammonium citrate, meat peptone, sodium chloride, pancreatic digest of casein, peptic digest of animal tissue, porcine brain heart infusion, potassium phosphate, sodium pyruvate, or yeast extract.

In some aspects, media may contain a pH buffer which may be a non-Magnesium chelating buffer. In some aspects, the pH buffer is a mixture of MOPS sodium salt and MOPS free acid, but a range of other buffers such as Carbonate and Phosphate buffers may be useable in alternative embodiments and will be readily selected amongst and implemented by those skilled in the art, to achieve a desired pH for the medium.

In some aspects, the media may be provided in the form of a powder or concentrate, also generally referred to as “powdered medium”, “medium powder”, “medium concentrate”, “concentrated medium” or the like, comprising a plurality of components and suitable to be combined with a predetermined volume of water to provide a liquid medium with desired concentrations of the particular components. Such a powdered medium or concentrated medium may be complete, meaning that it need only be dissolved in suitable water, normally sterile water, before use. Alternatively, in some aspects, a powdered or concentrated medium may be partial, meaning that additional components need to be added to provide a complete medium suitable for use. In embodiments a powdered or concentrated medium also includes medium that is at least partly hydrated in concentrated form suitable for dilution to produce the medium for actual use in culturing. It will be understood that term “medium” or “media” as used herein, unless otherwise required by the context, includes both the final media having components at concentrations suitable for culturing pathogens, and powdered or concentrated media suitable for dilution.

In some aspects, the components included in an enrichment solution include one or more of MOPS, Fe(111) salt, Lithium salt, pyruvate. In some aspects a selective enrichment supplement comprises one or more selective agents such as nalidixic acid, cycloheximide, and acriflavine hydrochloride. In some aspects, the enriched broth contains one or more of Magnesium sulphate, Lithium Chloride, Ferric Citrate, Sodium pyruvate and enrichment supplement.

In some aspects, the media may comprise one or more of Brain Heart Infusion Broth, Tryptic Soy Broth, Brucella Agar, Buffered Listeria Enrichment Broth Base, Carbohydrate Consumption Broth, Fraser Broth, Base, Fraser secondary enrichment broth base, HiCrome™ Listeria Agar Base, LPM Agar, Listeria Enrichment Broth according to FDA/IDF-FIL, Listeria Motility Medium, Listeria Selective Agar, Listeria mono Confirmatory Agar (Base), Listeria mono Differential Agar (Base), Nutrient Agar, Nutrient Broth No. 1, Nutrient Broth No. 2, Nutrient Broth No. 4, Oxford Agar, PALCAM Listeria Selective Agar, PALCAM Listeria Selective Enrichment Broth, Plate Count Agar, Plate Count Agar, Plate Count MUG Agar, Plate Count Skim Milk Agar, Rhamnose Broth, Tryptone Soya Yeast Extract Agar, UVM Listeria Selective Enrichment Broth, Universal Pre-Enrichment or a combination thereof.

In some aspects, the media may comprise one or more of Andrade Peptone Water, Andrade peptone water, Blood Agar (Base), Bromcresol Purple Broth, China Blue Lactose Agar, Christensen's Urea Agar, CLED Agar, Decarboxylase Broth Base, Moeller, DEV Lactose Broth, DEV Lactose Peptone Broth, DEV Tryptophan Broth, Glucose Bromcresol Purple Agar, HiCrome™ ECC Agar, HiCrome™ MM Agar, HiCrome™ UTI Agar, modified, Kligler Agar, Lactose Broth, Lactose Broth, Lactose Broth, Vegitone, Lysine Iron Agar, Malonate Broth, Methyl Red Voges Proskauer Broth, Methyl Red Voges Proskauer Saline Broth, Mineral-modified Glutamate Broth (Base), Motility Test Medium, Mucate Broth, MUG Tryptone Soya Agar, Nitrate Broth, OF Test Nutrient Agar, Simmons Citrate Agar, Triple Sugar Iron Agar, Tryptone Medium, Tryptone Water, Tryptone Water, Vegitone, Urea Broth Selective media for differentiation, BRILA MUG Broth, DEV ENDO Agar, ECD MUG Agar, EMB Agar, Endo Agar, ENDO Agar (Base), Gassner Agar, HiCrome™ Coliform Agar, HiCrome™ E. coli Agar B, HiCrome™ ECC Selective Agar, HiCrome™ ECD Agar with MUG Selective media for differentiation, HiCrome™ Mac Conkey Sorbitol Agar, HiCrome™ M-TEC Agar, HiCrome™ Rapid Coliform Broth, Lactose TTC Agar with Tergitol®-7, Levine EMB Agar, LST-MUG Broth, Mac Conkey Agar No. 1, Mac Conkey Agar No. 1, Vegitone, MacConkey Agar with Crystal Violet, Sodium Chloride and 0.15% Bile Salts, MacConkey Agar with Crystal Violet, Sodium Chloride and 0.15% Bile Salts, MacConkey Broth, MacConkey Broth purple, MacConkey MUG Agar, MacConkey-Agar (without salt), MacConkey-Sorbitol Agar, Membrane Lactose Glucuronide Agar, m-Endo Agar LES, M-FC Agar, m-FC Agar Plates (55 mm diameter), M-FC Agar, Vegitone, M-Lauryl Sulphate Broth, MUG EC Broth, TBX Agar, Tergitor-7 Agar, Violet Red Bile Agar, Violet Red Bile Agar, Vegitone, Violet Red Bile Glucose Agar, Violet Red Bile Glucose Agar without Lactose, Violet Red Bile Glucose Agar without Lactose, Vegitone, Violet Red Bile Lactose Dextrose Agar, VRB MUG Agar, WL Differential Agar, XLT4 Agar (Base) Selective media, A1 Broth, Brilliant Green Bile Lactose Broth, EC Broth, ECD Agar, Lauryl sulphate Broth, Lauryl sulphate Broth, M Endo Broth, M HD Endo Broth with Brilliant Green, M-Lauryl Sulphate Broth, Vegitone, Mossel Broth or a combination thereof.

In some aspects, the media comprises one or more of Bismuth sulfite Agar, BPL Agar, Brilliant Green Agar, modified, Brilliant Green Phenol Red Lactose Sucrose Agar, Purple Broth, DCLS Agar, DCLS Agar No. 2, Deoxycholate Citrate Agar, Glucose Hektoen Enteric Agar, Kligler Agar Fluka, Leifson Agar, Lysine Decarboxylase Broth, Muller-Kauffmann Tetrathionate Broth, Base (ISO), Pril® Mannitol Agar, Rappaport Vassiliadis Broth, Rappaport Vassiliadis Broth, modified, Rappaport Vassiliadis Medium, Rappaport Vassiliadis medium (base), modified, semi-solid, Salmonella Agar, Salmonella Enrichment Broth, Selenite Broth (Base), Selenite Cystine Broth, SIM Medium, SS-Agar, TBG Broth, Tetrathionate Broth, Tetrathionate Enrichment Broth, Triple Sugar Iron Agar, Urea Broth, XLD Agar or a combination thereof.

In some aspects, the media may comprise an oxygen scavenger. In some aspects, the oxygen scavenger may be selected from at least one of a pyruvate salt, catalase, a thioglycolate salt, cysteine, Oxyrase™, Na₂S, or FeS. In some aspects, the media may comprise from about 1.0 to about 20.0 g/L sodium pyruvate. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25 or more than about 25.0 g/L oxygen scavenger. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25 or more than about 25.0 g/L sodium pyruvate. In some aspects, the media may further comprise carbohydrate such as dextrose, esculin, maltose, amygdalin, cellobiose, fructose, mannose, salicin, dextrin, (x-methyl-D-glucoside and mixtures thereof. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30 or more than about 30.0 g/L carbohydrate. In some aspects, the media may comprise from about 1.0 to about 20.0 g/L dextrose. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 0.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 or more than about 20.0 g/L dextrose. In some aspects, the media may comprise Yeast Extract. In some aspects, the media may comprise from about 1.0 to about 30.0 g/L Yeast Extract. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30 or more than about 30.0 g/L Yeast Extract. In some aspects, the media may comprise salts such as sodium, potassium, or calcium salts of chloride. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4.7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 1.10, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 1.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30 or more than about 30.0 g/L salts. In some aspects, the media may comprise from about 1.0 to about 30.0 g/L sodium chloride. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30 or more than about 30.0 g/L sodium chloride. In some aspects, the media further comprises a protein, which may be provided from a variety of sources. For example, the protein may be provided from sources such as Tryptone, Tryptose, Soytone, Peptone, Pantone, Bitone, Proteose Peptone, pancreatic digest of gelatin, pancreatic digest of casein, enzymatic digest of soy and mixtures thereof. In some aspects, the media comprises from about 1.0 to about 70.0 g/L protein. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 1.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30, 31, 32, 33, 34, 35, 36, 37, 38, 39, 40, 45, 50, 60, 70 or more than about 70.0 g/L protein. In some aspects, the media comprises from about 1.0 to about 60.0 g/L pancreatic digest of casein. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30, 31, 32, 33, 34, 35, 36, 37, 38, 39, 40, 45, 50, 60 or more than about 60.0 g/L pancreatic digest of casein. In some aspects, the media comprises from about 1.0 to about 40.0 g/L enzymatic digest of soy. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5:1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30, 31, 32, 33, 34, 35, 36, 37, 38, 39, 40, or more than about 40.0 g/L pancreatic digest of casein. In some aspects, may further comprise buffers, which are effective for maintaining the pH in a desired range. For example, buffers that may be used include buffers such as potassium phosphate monobasic, potassium phosphate dibasic, sodium phosphate dibasic, and mixtures thereof. In some aspects, the media comprises from about 1.0 to about 50.0 g/L buffer. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 29, 30, 31, 32, 33, 34, 35, 36, 37, 38, 39, 40, 45, 50, or more than about 50.0 g/L buffers. In some aspects, the media may comprise from about 1 to about 20 g/L potassium phosphate. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0 or more than about 20.0 WI, potassium phosphate. In some aspects, the media comprises from about 1 to about 40 g/L di sodium phosphate. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 121, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0, 20.5, 21.0, 21.5, 22.0, 22.5, 23.0, 24.0, 25, 26, 27, 28, 2, 30, 31, 32, 33, 34, 35, 36, 37, 38, 39, 40, or more than about 40.0 g/L disodium phosphate. In some aspects, the media comprises from about 1 to about 20 g/L dipotassium phosphate. In some aspects, the media comprises about 0.0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1.0, 1 0.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.5, 14.0, 14.5, 15.0, 15.5, 16.0, 16.5, 17.0, 17.5, 18.0, 18.5, 19.0, 19.5 20.0 or more than about 20.0 g/L: dipotassium phosphate.

In some aspects, the media may comprise essential ions such as magnesium and/or iron. In some embodiments, magnesium may be selected from the group of magnesium sulfate, magnesium chloride, and mixtures thereof. In some embodiments, iron may be selected from the group of ferric ammonium citrate, ferrous sulfate, ferric sulfate, ferric citrate, ferrous ammonium sulfate, ferric chloride, and mixtures thereof.

In this disclosure the term “pyruvate salt” means and includes all salts of pyruvic acid (also known as 2-oxo-propanoic acid) and any compounds comprising a pyruvate anion, and any biologically effective isomers or substituted forms thereof. In embodiments the pyruvate salt is sodium or potassium pyruvate. Those skilled in the art will readily identify and avoid salts which are not biologically effective or desirable, for example due to toxicity. In embodiments a salt is soluble and may be organic or inorganic, and by way of example may be chloride, phosphate, nitrate, hydrogen carbonate, pyruvate, ethanoate.

In some embodiments, the yeast extract may be yeast autolysate or yeast hydrolysate. For example, the yeast extract may include the water-soluble compounds of yeast autolysate. In this regard autolysis of the yeast cells may be carefully controlled to preserve natural vitamin B complexes. The yeast extract may be obtainable by growing Saccharomyces spp. in carbohydrate-rich plant media. The yeast may be harvested, washed and resuspended in the water, and then self-digested with its own enzymes (“autolysis”) in the water. The autolytic activities of the enzymes may be lost by heating. The resulting yeast extract is filtered until it becomes clear, and the filtrate is spray-dried into powder form. The yeast extract may supply vitamins, nitrogen, amino acids, and carbon to the medium. The yeast extract may be commercially available from, for example, DIFCO™ Laboratories Inc., and ACUMEDIA™ Inc.

In some embodiments, the media may comprise suitable carbon sources. Among the suitable carbon sources are, for example, glucose, fructose, xylose, sucrose, maltose, lactose, mannitol, sorbitol, glycerol, corn syrup and corn syrup solids. Examples of suitable nitrogen sources include organic and inorganic nitrogen-containing substances such as peptone, corn steep liquor, meat extract, yeast extract, casein, urea, amino acids, ammonium salts, nitrates, enzymatic digest of soy, and mixtures thereof.

Those skilled in the art will readily understand that the growth of a desired microorganism will be best promoted at selected temperatures suited to the microorganism in question. In particular embodiments culturing may be carried out at about 39° C. and the media to be used may be pre-warmed to this temperature. In embodiments disclosed herein, enrichment may be carried out at any temperature between 33° C. and 43° C. and may be carried out at about 33° C., 34° C., 35° C., 36° C., 37° C., 38° C., or 39° C., or 40° C., or 41° C. or 42° C. or 43° C. or between 33° C. and 34° C., 34° C. and 35° C. 35° C. and 36° C., 36° C. and 37° C., 37° C. and 38° C., 38° C. and 39° C., 39° C. and 40° C., 40° C. and 41° C., 41° C. and 42° C., or 42° C. and 43° C. or at a temperature of between 34° C. and 43° C., or between 35° C. and 42° C., or between 36° C. and 42° C., 38° C. and 42° C. or between 39° C. and 41° C. or between 39° C. and 40° C. or between 38° C. and 39° C., or between 39° C. and 40° C., or between 40° C. and 41° C., or between 41° C. and 42° C. or between 42° C. and 43° C. In some embodiments, the kits described herein can comprise a media disclose herein. In some embodiments, the kits disclosed herein can comprise Media A or a derivative thereof. In some embodiments, the kits disclosed herein can comprise Media B or a derivative thereof. In some embodiments, Media A comprise: yeast extract 6 g/L, pancreatic digest of casein 17 g/L, enzymatic digest of soy 3 g/L, dextrose 2.5 g/L, NaCl 5 g/L, dipotassium phosphate 2.5 G/L, potassium phosphate 1.35 g/L, disodium phosphate 9.6 g/L, sodium pyruvate 1.1 g/L. In some embodiments, Media A can have a pH within the range of 7.2-7.4. In some embodiments, Media B comprise: yeast extract 12 g/L, pancreatic digest of casein 34 g/L, enzymatic digest of soy 6 g/L, dextrose 5 g/L, NaCl 10 g/L, dipotassium phosphate 5 g/L, potassium phosphate 2.7 g/L, disodium phosphate 19.2 g/L, sodium pyruvate 2.2 g/L. In some embodiments, Media B can have a pH was within the range of 7.2-7.4. In some embodiments, a media can comprise of beef heart solids 0-8 g/L, calf brain solids 0-10 g/L, calf brain-beef heart infusion 0-35 g/L, casein peptone 0-16 g/L, dextrose 0-10 g/L, dipotassium phosphate 0-7 g/L, disodium phosphate 0-20 g/L, enzymatic digest of soy 0-8 g/L, esculin 0.5-3 g/L, ferric ammonium citrate 0-10 g/L, meat peptone 0-8 g/L, sodium chloride 0-10 g/L, pancreatic digest of casein 0-35 g/L, peptic digest of animal tissue 0-10 g/L, porcine brain heart infusion 0-12 g/L, potassium phosphate 0-5 g/L, sodium pyruvate 0-4 g/L, or yeast extract 0-14 g/L. In some embodiments the media can have selective agents acriflavine hydrochloride 0-1 g/L, cycloheximide 0-1 g/L, lithium chloride 0-10 g/L or nalidixic acid 0-1 g/L.

Supplement

In some embodiments, the media comprises supplements.

In some embodiments, a supplement comprises one or more of a magnesium salt, a lithium salt, an iron(lll) salt, a pyruvate and a selective agent, or comprises precursors or modified forms that may be readily converted or metabolized to form any of the foregoing. In some embodiments, a supplement is a supplement for promoting the growth of one or more pathogen. In some embodiments, a supplement is a supplement for promoting the growth of Listeria spp. In some embodiments, selective agents include antibiotics, sulphanamides or antiseptics. In some embodiments, a selective agent is or comprises one, two or all three of nalidixic acid, cycloheximide and acriflavine hydrochloride or includes suitable equivalents or alternatives thereto. In some embodiments, the working concentration of cycloheximide is about 33.75 mg per liter of culture medium, and in alternative embodiments is between 15 and 50 mg/liter of culture medium, or may be greater than 5, 0, 15, 20, 25, 30, 35, 40, 45, 50, 55, 60, 65, 70, 75, 80, 85, 90, 95, 100 or more mg/liter of culture medium. In some embodiments the working concentration of nalidixic acid is about 27 mg per liter of culture medium, or is between 10 and 50 mg/liter of culture medium or is greater than 5, 10, 15, 20, 25, 30, 35, 40, 45, 50, 55, 60, 65, 70, 75, 80, 85, 90, 95, 100 or more mg/liter of culture medium. In some embodiments, the working concentration of acriflavine hydrochloride is about 10, 25 mg/liter, or is between 6000 and 15,000 mg/liter, or is greater than 1000, 2000, 3000, 4000, 5000, 6000, 7000, 8000, 9000, 10000, 11000, 12000, 13000, 14000, 15000, 16000, 17000, 18000, 19000, 20000 or more mg/liter of culture medium. Those skilled in the art will, however, recognize that a wide variety of concentrations of selective agents may be employed and will make suitable adjustments for particular purposes.

In some embodiments, the media is free of supplements.

In some embodiments, the media is substantially free of supplements.

pH

In some aspects, the pH of culture medium is generally set at between 7 and 8 and for example in particular embodiments may be about 7.0, 7.1, 7.2, 7.3, 7.4, 7.5, 7.6, 7.7, 7.8, 7.9 or 8.0 or is in a range delimited by any two of the foregoing values. It will be understood that a pH outside of the range pH7-8 may still be useable in embodiments, but that the efficiency and selectivity of the culture may be adversely affected. Therefore in some embodiments, the pH can be 1-7 or 8-14.

Volume

In some embodiments, the sample may be enriched by suspension in media at a volume in the range of about 10 ml to about 1000 ml. In some embodiments, the sample maybe enriched by suspension in media at a volume of about 10 ml, 20 ml, 30 ml, 40 ml, 50 ml, 60 ml, 70 ml, 80 ml, 90 ml, 100 ml, 110 ml, 120 ml, 130 ml, 140 ml, 150 ml, 160 ml, 170 ml, 180 ml, 190 ml, 200 ml, 210 ml, 220 ml, 225 ml, 230 ml, 240 ml, 250 ml, 300 ml, 350 ml, 400 ml, 450 ml, 500 ml, 550 ml, 600 ml, 650 ml, 700 ml, 800 ml, 900 ml, or about 1000 ml, in some embodiments a volume above 50 ml, in some embodiments the volume is about 225±10 ml, in some embodiments about 225 ml.

Homogenizing

In some embodiments, the sample may be homogenized or otherwise finely divided in order to separate the pathogen from the sample by techniques know to one of skill in the art. For example, stirring, mixing, agitating, blending, or vortexing. In some embodiments, samples may be homogenized by hand mixing, stomaching, or blending. In some embodiments, the sample is stomached. A stomaching device can be used that mixes a source and diluents in a bag through the use of two paddles in a kneading-type action. See, for example, U.S. Pat. No. 3,819,158. An oscillating device known as the PULSIFIER® is described in U.S. Pat. No. 6,273,600, which employs a bag placed inside an agitating metal ring. Another technique, vortexing for analyte suspension, has been described in U.S. Pat. No. 6,273,600. See also U.S. Patent.

In some embodiments, the sample may be homogenized for about 1, 2, 3, 4, 5, 6, 7, 8, 9, 10, 15, 20, 25, 26, 27, 28, 29, 30, 31, 32, 33, 34, 35, 40, 45, 50, 55, 60, 70, 80, 90, 100, 110, 120, 130, 140, 150, 160, 170, 180, 190, 200, 210, 220, 225, 230, 240, 250, 300, 350, 400, 450, 500, 550, 600, 650, 700, 800, 900, or 1000 seconds, in some embodiments above 15 seconds, in some embodiments 30±5 seconds, in some embodiments about 30 seconds.

Following homogenization, in some embodiments, the sample may be incubated. For example, incubation following homogenization may occur at a temperature of about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80° C., or to any other temperature above 80° C. In some embodiments, the incubation temperature is in the range of about 25 to about 80° C., in some embodiments about 25 to about 45° C., in some embodiments the temperature is about 37±5° C. In some embodiments, incubation following homogenization can be for a time period in the range of about 1 minute to about 48 hours, e.g., 1, 5, 10, 15, 20, 25, 30, 40, 50, 60, 70, 80, 90, 100, 110, 120, 130, 140, 150, 160, 170, 180, 190, 200, 210, 220, 230, 240, 250, 260, 270, 280, 290, 300, 400, 500, 600, 700, 800, 900, 1000, 1500, 2000, 2500, 3000 minutes, in some embodiments 60 minutes. In some embodiments, the sample is incubated following homogenization while being agitated. In some embodiments, the sample is incubated following homogenization and is agitated at a speed in the range of 20 to 3500 rpm, e.g., 20, 50, 100, 150, 200, 300, 400, 500, 700, 1000, 1100, 1200, 1300, 1350, 1400, 1450, 1500, 1550, 1600, 1750, 2000, 2250, 2500, 2750, 3000, 3250, 3500 rpm. In some embodiments, the sample can be incubated following homogenization while substantially free of being agitated. In some embodiments, the sample can be incubated following homogenization free of being agitated.

Lysis

Cell lysis is a process of releasing materials in a cell by disrupting the cell membrane, and in particular, a process of extracting intracellular materials from a cell to isolate DNA or RNA before amplification, such as a polymerase chain reaction (PCR). In some embodiments, cell lysis may be performed to isolate DNA or RNA before amplification, such as a polymerase chain reaction (PCR).

In some aspects, lysis may be by mechanical methods include ultrasonication, disruption using a homogenizer, pressing mechanism, for example, a French press, etc., decompression, pulverization, etc. Non-mechanical lysis methods include chemical methods, thermal methods, enzymatic methods, etc.

In some aspects, nucleic acid from the sample may be isolated using known techniques. For example, the sample may be treated to lyse the cells, using known lysis buffers, sonication, electroporation, etc., with purification occurring as needed, as will be appreciated by those in the art. In addition, the reactions outlined herein may be accomplished in a variety of ways, as will be appreciated by those in the art. Components of the lysis reaction may be added simultaneously, or sequentially, in any order, with some embodiments outlined below. In some aspects, the lysis reaction may include a variety of other reagents that may be included in assays to be performed following cell lysis. In some aspects, these reagents include salts, buffers, neutral proteins, e.g. albumin, detergents, etc., which may be used to facilitate optimal hybridization and detection, and/or reduce non-specific or background interactions. In some aspects, reagents that otherwise improve the efficiency of an assay, such as protease inhibitors, nuclease inhibitors, anti-microbial agents, etc., may be used, depending on the sample preparation methods and purity of the target.

In some aspects, lysis may be by a lysis buffer. In some aspects, the lysis buffer has a pH that is approximately neutral. In some aspects, the lysis buffer has a pH in the range of 5.5 to 8, i.e., a pH of 5.5, 6, 6.5, 7, 7.5, or 8, in some aspects a pH of about 7. It will be understood that a pH outside of the range pH7-8 may still be useable in embodiments. Therefore in some aspects, the pH can be about 1-7 or about 8-14.

Recovery of DNA and/or RNA utilizing a lysis buffer of the present invention may proceed by combining the lysis buffer sample, agitating the mixture of the cells and lysis buffer to provide a mixture including a supernatant including DNA and/or RNA to be recovered and a solids fraction, and recovering the DNA-containing supernatant.

In some aspects, a portion of the sample may be combined with the lysis buffer and forms a sample/lysis buffer mixture. In some aspects, formation of the sample/lysis buffer mixture includes dilution of the lysis buffer with an aqueous medium (e.g., deionized water). In some aspects, an aqueous medium is combined with the lysis buffer at a volumetric ratio (aqueous medium:lysis buffer) of about 1:1, 1:2, 1:3, 1:4, 1:5, 1:6, 1:10, 1:20, 20:1, 10:1, 6:1, 5:1, 4:1, 3:1, or about 2:1 for dilution. After the sample and lysis buffer have been combined, the mixture is treated to provide breakdown of the sample cell walls and release of DNA and/or RNA. In some aspects, this treatment includes agitation of the sample/lysis buffer mixture, which generally includes placing samples of the mixture into a suitable container (e.g., a multi-well plate, deep-well block) and shaking of the samples.

In some aspects, the agitation for breakdown of cell walls and release of DNA and/or RNA includes contacting the sample with particulate matter for facilitating breakdown of the cell walls. In some aspects, this contact generally includes placing suitable particulate matter in each well of the multi-well plate/deep-well block so that the particulate matter and sample come into mutually abrading contact during agitation (e.g., shaking) of the sample/lysis buffer mixture. The particulate matter is generally spherical and constructed of suitable material (e.g., stainless steel). In some aspects, the particulate matter may not be spherical.

In some aspects, after a suitable period of agitation of the sample/lysis buffer mixture, the resulting mixture generally comprises a lysed sample mixture including a solids fraction and a supernatant comprising nucleic acid to be recovered. In some aspects, the lysed sample may be treated for purposes of separating the solids fraction and supernatant. In some aspects, this treatment generally comprises centrifuging the samples (i.e., the multi-well plate, deep-well block) under suitable conditions. Typically, the samples are subjected to treatment by centrifuging at from about 1000 to about 3500 revolutions per minute (rpm) for from about 5 to about 10 minutes.

In some aspects, prior to agitation of the sample/lysis buffer mixture, the mixture may be subjected to an incubation period. In some aspects, the incubation period proceeds for at least about 5 minutes, at least about 10 minutes, or at least about 15 minutes. In some aspects, during the incubation period, the sample/lysis mixture may be subjected to temperatures of room temperature, or even higher. In some aspects, the sample/lysis mixture may be subjected to temperatures of up to about 25° C., up to about 35° C., or up to about 45° C., or up to about 55° C., or up to about 65° C., or up to about 75° C. The precise combination of time/temperature incubation conditions is not narrowly critical, however, in various embodiments, the incubation proceeds for a up to about 15 minutes while the sample/lysis buffer mixture is subjected to a temperature of from about 20° C. to about 30° C. (e.g, about 25° C.).

In some aspects, separation of the lysed sample mixture (e.g., by centrifuging) forms a lysed sample mixture including a nucleic acid supernatant that is then recovered from the lysed sample mixture. In some aspects, the nucleic acid is then subjected to analysis by any method known in the art, including but not limited to those listed below. In some aspects, the DNA content of the lysed sample mixture and/or the nucleic acid supernatant is at least about 50%, at least about 60%, at least about 70%, at least about 80%, at least about 90%, at least about 95%, or at least about 99% of the DNA present in the sample prior to lysing the sample.

In some aspects, the lysis buffer comprises a buffering component. In some aspects, the lysis buffer according to the present invention may comprise buffering components that may, for example, be used to adjust the pH of the lysis buffer. In some aspects, buffering components include, for example, 3-{[tris(hydroxymethyl)methyl]amino}propanesulfonic acid (TAPS), N,N-bis(2-hydroxy-ethly)glycine (Bicine), tris(hydroxymethyl)methylamine (TRIS), N-tris(hydroxylmethyl)-methylglyxine (Tricine), 4-2-hydroxyethyl-1-piperazineethanesulfonic acid (HEPES), 2-{[tris(hydroxymethyl)methyl] amino}ethanesulfonic acid (TES), 3-(N-morpholino)propane-sulfonic acid (MOPS), piperazine-N,N′-bis(2-ethanesulfonic acid) (PIPES), dimethylarsinic acid (Cacodylate), saline sodium citrate (SSC), 2-(N-morpholino)ethanesulfonic acid (MES), and combination thereof. In some aspects, the lysis buffer according to the present invention may comprise buffering components present at a concentration in the range of about 0.01 to about 300 mM. In some aspects, the buffering components can be present at about 0.01 mM, 0.05 mM, 0.1 mM, 0.5 mM 1.0 mM, 5.0 mM, 10 mM, 20 mM, 30 mM, 40 mM, 50 mM, 60 mM, 70 mM, 80 mM, 90 mM, 110 mM, 120 mM, 130 mM, 140 mM, 150 mM, 160 mM, 170 mM, 180 mM, 190 mM, or 200 mM, 250 mM, or about 300 mM. In some aspects, the buffering components can be present at a concentration above about 20 mM. In some aspects, the concentration is above 20 mM, in some aspects, the concentration is about 80±10 mM, in some aspects, the concentration is about 80 mM. In some aspects, the lysis buffer is substantially free of a buffering component.

In some aspects, the lysis buffer according to the present invention may comprise chelating agents. In some aspects, the chelating agents include, for example, acetylacetone, ethylenediamine, diethylenetriamine, iminodiacetate, triethylenetetramine, triaminotriethylamine, nitrilotriacetate, ethylenediaminotriacetate, ethylenediaminotetraacetic acid (EDTA), ethylene glycol tetraacetic acid (EGTA), diethylene triamine pentaacetic acid (DTP A), 1,4,7,10-tetraazacyclododecane-1,4,7,10-tetraacetic acid (DOTA), and combination thereof. In some embodiments, the lysis buffer according to the present invention, contain one or more chelating agents, for example, one or more of the above chelating agents. In some aspects, the lysis buffer may contain one or more chelating agents in a concentration in the range of about 0.5 to about 100 mM, in some aspects, about 5 to about 10 mM, such as in a concentration of about 1, 2, 5, 6, 7, 8, 9, 10, 15, 20, 25, 30, 35, 40, 45, 50, 60, 70, 80, 90, or 100 mM. In some aspects, the lysis buffer is substantially free of a metal chelating agent.

In some aspects, the lysis buffer may comprise a surfactant. In some aspects, the surfactants include, for example, alkyl sulfate salts, such as sodium dodecyl sulfate (SDS) or ammonium lauryl sulfate, non-ionic surfactants, such as Triton X-100, octyl glucoside, Genapol X-100, or polysorbates, e.g., Tween 20 or Tween 80, and sarkosyl (N-lauroyl-sarcosine) and combinations thereof. In some aspects, surfactant of the present invention may also include nonyl phenoxypolyoxylethanol (NP-40). In some aspects, the lysis buffer according to the present invention may contain one or more chelating agents, for example, one or more of the above surfactants. In some aspects, the lysis buffer according to the present invention may contain one or more surfactants in a concentration in the range of about 0.2% to about 20% (w/v), in some aspects, about 0.5% to 10% (w/v), such as about 0.5, 1.0, 1.5, 2.0, 2.5, 3.0, 3.5, 4.0, 4.5, 4.8, 5.0, 5.5, 6.0, 6.5, 7.0, 7.5, 8.0, 8.5, 9.0, 9.5, or about 10% (w/v). In some aspects, the lysis buffer according to the present invention is substantially free of surfactants.

In some aspects, the lysis buffer may comprise a precipitant. In some aspects, precipitants include, for example, glycerol, dimethyl sulfoxide (DMSO), acetonitrile (ACN), bovine serum albumin (BSA), proteinase K, acetate salts, and combinations thereof. In some aspects, the lysis buffer can comprise proteinase K. In some aspects, the lysis buffer according to the present invention may contain one or more precipitants in a concentration in the range of about 2% to about 50% (w/v), in some aspects, about 15% to about 35% (w/v), such as about 2, 5, 10, 15, 20, 21, 22, 23, 24, 25, 26, 27, 28, 29, 30, 35, 40, 45, 48, 50, 55, or about 60% (w/v). In some aspects, the lysis buffer according to the present invention is substantially free of precipitants.

In some aspects, the lysis buffer may comprise a lysing moiety. In some aspects, the lysis buffer comprises at least two lysing moieties. In some aspects, the lysing moiety is a bead. In some aspects, the beads may exhibit any shape, for example, the bead may be ball-shaped, cube-shaped, triangular-shaped, or they may exhibit any irregular shape. In some aspects, the beads are made of a solid inert material. In some aspects, the beads exhibit a firm consistency and do not react chemically with biological substances such as proteins or nucleic acids to a significant extent. In some aspects, the beads do not bind nucleic acids to a significant extent. In some aspects, the beads are made of glass, ceramics, plastics, or metal such as steel. In some aspects, bead surfaces may be made to a variety of bead types including, but not limited to, beads made with silica (e.g., manufactured as fused quartz, crystal, fumed silica or pyrogenic silica, colloidal silica, silica gel, aerogel, glass, fiber (e.g., optical fiber), cement and ceramics (e.g., earthenware, stoneware, and porcelain), zirconium, zirconium silica, zirconium yttrium, and all other related glass oxide and mixtures of glasses and oxides. In some aspects, the term “beads” does not refer beads used for nucleic acid isolation. In some aspects the term “beads” as described herein and are present in the lysis reaction mixture in a concentration in the range of about 0.50 to about 1.5 g/ml, in some aspects, in the range of about 0.100 to about 0.900 g/ml, in some aspects, in the range of about 0.150 to about 0.950 g/ml, in some aspects, in the range of about 0.250 to about 0.950 g/ml. In some embodiments, beads may be present in the lysis buffer mixture in a concentration of about 0.50, 0.100, 0.150, 0.200, 0.250, 0.300, 0.350, 0.400, 0.450, 0.500, 0.600, 0.700, 0.800, 0.850, 0.88, 0.900, 1, 1.1, 1.2, 1.3, 1.4, or about 1.5 g/ml, in some aspects, in a concentration of about 0.8±0.1 g/ml, in some aspects, in a concentration of about 0.88±0.05 g/ml.

In some aspects, the lysing moiety may be a lysing enzyme. In some aspects, the lysing moiety may be β-glucuronidase, Mutanolysin, lysozyme, Achromopeptidase, Lysostaphin, Labiase, combination thereof and/or other lytic enzymes know by one of skill in the art. In some aspect, the lysing moiety may be lysozyme. In some aspects, lysozyme as described herein are present in the lysis buffer in a concentration in the range of about 5 to about 150 mg/ml, in some aspects, in the range of about 15 to about 25 mg/ml, in some aspects, in the range of about 18 to about 25 mg/ml, in some aspects, in the range of about 20 to about 25 mg/ml. In some aspects, lysozyme may be present in the lysis buffer in a concentration of about 5, 10, 15, 20, 0.25, 30, 35, 40, 45, 50, 60, 70, 80, 85, 90, 100, 110, 120, 130, 140, or about 150 mg/ml, in some aspects, in a concentration of about 20±3 mg/ml, in some aspects, in a concentration of about 20±0.05 mg/ml.

In some aspects, the lysis buffer further comprises a mineral salt selected from the group consisting of sodium chloride (NaCl), potassium chloride (KCl), diammonium sulfate (NH₄SO₄), and combinations thereof.

In some aspects, the lysis buffer may further comprise sodium chloride (NaCl). In some aspects, the lysis buffer may further comprise potassium chloride (KCl). In some aspects, the lysis buffer may further comprise diammonium sulfate (NH₄SO₄).

In some aspects, the lysis buffer may further comprise an alkali metal hydroxide selected from the group consisting of sodium hydroxide, potassium hydroxide, and combinations thereof. In some aspects, the lysis buffer may further comprise sodium hydroxide. In some aspects, the lysis buffer may further comprise potassium hydroxide.

In some aspects, cell lysis can occur in one step.

In some aspects, cell lysis may occur in two or more steps. In some aspects, cell lysis can occur in two steps. In some aspects, the first sample lysis may comprise combining the sample and the lysis buffer composition thereby forming a sample/lysis buffer mixture, agitating the sample/lysis buffer mixture, thereby lysing the sample and forming a lysed sample mixture. In some aspects, a second sample lysis comprising continuing to agitate the lysed sample mixture is performed at a temperature higher than the temperature of the first sample lysis. In some aspects, the first sample lysis may be performed at a first temperature, for example, the sample/lysis buffer mixture is heated from room temperature to about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80° C., or to any other temperature above about 80° C. In some aspects, the first temperature is in the range of about 25 to about 80° C., in some aspects, about 40 to about 70° C., in some aspects, the first temperature is about 65±5° C. In some aspects, the second sample lysis may be performed wherein the sample/lysis buffer mixture/lysed sample mixture is heated to a second temperature. In some aspects, the second temperature is higher than the first temperature. In some aspects, the second temperature is in the range of about 50 to about 120° C., in some aspects, about 60 to about 100° C., in some aspects, about 80 to about 100° C. In some aspects, the second temperature may be about 80, 81, 82, 83, 84, 85, 86, 87, 88, 89, 90, 91, 92, 93, 94, 95, 96, 97, 98, 99, or about 100° C., in some aspects, about 95±5° C. In some aspects, the difference between the first temperature and the second temperature may be about 1, 5, 10, 15, 20, 25, 30, 35, 40, 45, 50, 60, 70, 80, 90, or 100° C. In some aspects, the difference between the first temperature and the second temperature may be in the range of about 1-5, 5-10, 10-15, 15-20, 20-25, 25-30, 30-35, 35-40, 40-45, 45-50, 50-60, 60-70, 70-80, 80-90, or 90-100° C. In some aspects, following the second sample lysis, the temperature is reduced to about room temperature.

In some aspects, the first sample lysis may occur for a time period in the range of about 1 minute to about 1 hour, e.g., about 1, 5, 10, 15, 20, 25, 30, 40, 50, or about 60 minutes, in some aspects, about 15 minutes. In some aspects, the second sample lysis may occur for a time period in the range of about 1 minute to about 1 hour, e.g., about 1, 5, 10, 15, 20, 25, 30, 40, 50, or about 60 minutes, in some aspects, about 10 minutes. In some aspects, the first sample lysis and the second sample lysis is agitated at a speed in the range of about 1000 to about 3500 rpm, e.g., about 1000, 1100, 1200, 1300, 1350, 1400, 1450, 1500, 1550, 1600, 1750, 2000, 2250, 2500, 2750, 3000, 3250, or about 3500 rpm, in some aspects, at a speed of about 1350±100 rpm.

Reverse Transcription

In some embodiments, nucleic acid recovered following sample lysis may be DNA or RNA. In some embodiments, a method disclosed herein can be performed without extracting/isolating nucleic acids from the one or more pathogens. In some embodiments, a method disclosed herein can be performed without extracting/isolating nucleic acids from the one or more pathogens following lysis.

In some embodiments, nucleic acid is prepared from RNA by reverse transcription. In some embodiments, the nucleic acids are prepared from DNA by primer extension, such as using a polymerase.

In some embodiments, the methods described herein can be used in coupled reverse transcription-PCR (reverse transcription-PCR). In some embodiments, reverse transcription and PCR can be carried out in two distinct steps. In some embodiments, a cDNA copy of the sample mRNA can be synthesized using either a polynucleotide dT primer, a sequence specific primer, a universal primer, or any primer described herein.

In some embodiments, reverse transcription and PCR can be carried out in a single closed vessel reaction. For example, three primers can be employed, one for reverse transcription and two for PCR. In some embodiments, the primer for reverse transcription can bind to the mRNA 3′ to the position of the PCR amplicon. In some embodiments, the reverse transcription primer can include RNA residues or modified analogs such as 2′-O-methyl RNA bases, which will not form a substrate for RNase H when hybridized to the mRNA.

The temperature to carry out the reverse transcription reaction depends on the reverse transcriptase being used. In some embodiments, a thermostable reverse transcriptase is used and the reverse transcription reaction is carried out at about 37° C. to about 75° C., at about 37° C. to about 50° C., at about 37° C. to about 55° C., at about 37° C. to about 60° C., at about 55° C. to about 75° C., at about 55° C. to about 60° C., at about 37° C., or at about 60° C. In some embodiments, a reverse transcriptase that transfers 3 or more non-template terminal nucleotides to an end of the transcribed product is used.

In some embodiments, a reverse transcription reaction and the PCR reaction described herein can be carried out in various formats known in the art, such as in tubes, microtiter plates, microfluidic devices, or droplets.

In some embodiments, a reverse transcription reaction can be carried out in volumes ranging from about 5 μL to 500 μL, or in 10 μL to about 20 μL reaction volumes. In droplets, reaction volumes can range from about 1 pL to 100 nL, or 10 pL to about 1 nL. In some embodiments, the reverse transcription reaction is carried out in a droplet having a volume that is about or less than 1 nL.

In some embodiments, target polynucleotides, such as RNA, can be reverse transcribed into cDNA using one or more reverse transcription primers. In some embodiments, one or more reverse transcription primers can comprise a region complementary to a region of the RNA. In some embodiments, the reverse transcription primers can comprise a first reverse transcription primer with a region complementary to a region of a first RNA, and a second reverse transcription primer with a region complementary to a region of a second RNA. In some embodiments, the reverse transcription primers can comprise a first reverse transcription primer with a region complementary to a region of a first RNA, and one or more reverse transcription primers with a region complementary to a region of one or more RNAs, respectively.

In some embodiments, reverse transcription primers can further comprise a region that is not complementary to a region of the RNA. In some embodiments, the region that is not complementary to a region of the RNA is 5′ to a region of the primers that is complementary to the RNA. In some embodiments, the region that is not complementary to a region of the RNA is 3′ to a region of the primers that is complementary to the RNA. In some embodiments, the region that is not complementary to a region of the RNA is a 5′ overhang region. In some embodiments, the region that is not complementary to a region of the RNA comprises a priming site for amplification and/or a sequencing reaction. Using the one or more primers described herein, the RNA molecules are reverse transcribed using suitable reagents known in the art.

In some embodiments, the forward/reverse primers in the plurality of forward/reverse primers may further comprise a region that is not complementary to a region of the RNA. In some embodiments, the region that is not complementary to a region of the RNA is 5′ to a region of the forward/reverse primers that is complementary to the RNA. In some embodiments, the region that is not complementary to a region of the RNA is 3′ to a region of the forward/reverse primers that is complementary to the RNA. In some embodiments, the region that is not complementary to a region of the RNA is a 5′ overhang region. In some embodiments, the region that is not complementary to a region of the RNA comprises a priming site for amplification and/or a second sequencing reaction. In some embodiments, the region that is not complementary to a region of the RNA comprises a priming site for amplification and/or a third sequencing reaction. In some embodiments, the region that is not complementary to a region of the RNA comprises a priming site for a second and a third sequencing reaction. In some embodiments, the sequence of the priming site for the second and the third sequencing reaction are the same. In some embodiments, using the one or more forward/reverse primers and a reverse primer as described herein, the cDNA molecules are amplified using suitable reagents known in the art. In some embodiments, the primers comprises about 80%, 81%, 82%, 83%, 84%, 85%, 86%, 88%, 89, 90%, 91%, 92%, 93%, 94%, 95%, 96%, 97%, 98%, or at least about 99% sequence identity to the primers of Table 1.

Amplification

In some aspects, the nucleic acid recovered following cell lysis or the sample containing the one or more pathogens comprises fragments thereof, which can be amplified. In some aspects, the average length of the mRNA, or fragments thereof, can be less than about 100, 200, 300, 400, 500, or about 800 base, or less than about 5, 10, 20, 30, 40, 50, 60, 70, 80, 90, 100, 110, 120, 130, 140, 150, 160, 170, 180, 190, or 200 nucleotides, or less than about 1, 2, 5, 10, 20, 30, 40, 50, 60, 70, 80, 90, about 100 kilobases. In some aspects, a target sequence may be from a relative short template, such as a sample containing a template that is about 40, 45, 50, 55, 60, 65, 70, 75, 80, 85, 90, 95, or about 100 bases, is amplified.

In some aspects, an amplification reaction can comprise one or more additives. In some aspects, the one or more additives are dimethyl sulfoxide (DMSO), glycerol, betaine (mono)hydrate (N,N,N-trimethylglycine=[caroxy-methyl] trimethylammonium), trehalose, 7-Deaza-2′-deoxyguanosine triphosphate (dC7GTP or 7-deaza-2′-dGTP), BSA (bovine serum albumin), formamide (methanamide), tettrmethylammonium chloride (TMAC), other tetraalkylammonium derivatives (e.g., tetraethyammonium chloride (TEA-Cl) and tetrapropylammonium chloride (TPrA-Cl), non-ionic detergent (e.g., Triton X-100, Tween 20, Nonidet P-40 (NP-40)), or PREXCEL-Q. In some aspects, an amplification reaction can comprise 0, 1, 2, 3, 4, 5, 6, 7, 8, 9, or 10 different additives. In some aspects, an amplification reaction can comprise 10 or more different additives.

In some embodiments, thermocycling reactions can be performed on samples contained in reaction volumes.

In some aspects, the nucleic acid recovered following cell lysis or the sample containing the one or more pathogens can comprise cDNA, DNA, or fragments thereof, which can be amplified. In some aspects, the average length of the DNA, cDNA, or fragments thereof, can be less than about 100, 200, 300, 400, 500, or about 800 base pairs, or less than about 5, 10, 20, 30, 40, 50, 60, 70, 80, 90, 100, 110, 120, 130, 140, 150, 160, 170, 180, 190, or about 200 nucleotides, or less than about 1, 2, 5, 10, 20, 30, 40, 50, 60, 70, 80, 90, or about 100 kilobases. In some cases, a target sequence from a relative short template, such as a sample containing a template that is about 40, 45, 50, 55, 60, 65, 70, 75, 80, 85, 90, 95, or about 100 bases, is amplified.

In some aspects, any DNA polymerase that catalyzes primer extension can be used, including but not limited to E. coli DNA polymerase, Klenow fragment of E. coli DNA polymerase 1, T7 DNA polymerase, T4 DNA polymerase, Taq polymerase, Pfu DNA polymerase, Vent DNA polymerase, bacteriophage 29, REDTaq™, Genomic DNA polymerase, or sequenase. In some aspects, a thermostable DNA polymerase is used. In some aspects, a hot start PCR can also be performed wherein the reaction is heated to about 95° C. for two minutes prior to addition of the polymerase or the polymerase can be kept inactive until the first heating step in cycle 1. In some aspects, hot start PCR can be used to minimize nonspecific amplification. In some aspects, any number of PCR cycles can be used to amplify the DNA, e.g., about, more than about, or less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 100 cycles. The number of amplification cycles can be about 1-45, 10-45, 20-45, 30-45, 35-45, 10-40, 10-30, 10-25, 10-20, 10-15, 20-35, 25-35, 30-35, or about 35-40.

In some aspects, amplification of target nucleic acids can be performed by any means known in the art. In some aspects, target nucleic acids can be amplified by polymerase chain reaction (PCR) or isothermal DNA amplification. In some aspects, the amplification technique may be PCR. The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference.

In some aspects, examples of PCR techniques that can be used include, but are not limited to, quantitative PCR, quantitative fluorescent PCR (QF-PCR), multiplex PCR, multiplex fluorescent PCR (MF-PCR), real time PCR (reverse transcription-PCR), single cell PCR, restriction fragment length polymorphism PCR (PCR-RFLP), PCR-RFLP/reverse transcription-PCR-RFLP, hot start PCR, nested PCR, nested multiplex PCR, in situ polony PCR, in situ rolling circle amplification (RCA), digital PCR (dPCR), droplet digital PCR (ddPCR), bridge PCR, picotiter PCR and emulsion PCR. Other suitable amplification methods include the ligase chain reaction (LCR), transcription amplification, molecular inversion probe (MIP) PCR, self-sustained sequence replication, selective amplification of target polynucleotide sequences, consensus sequence primed polymerase chain reaction (CP-PCR), arbitrarily primed polymerase chain reaction (AP-PCR), degenerate polynucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938, as well as include Q beta replicase mediated RNA amplification.

In some aspects, amplification can be isothermal amplification, e.g., isothermal linear amplification.

In some aspects, examples of PCR that can be use in the invention include, but is not limited to “quantitative competitive PCR” or “QC-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”, “panhandle PCR”, and “PCR select cDNA subtraction”, “allele-specific PCR”, among others. In some aspects, the amplification technique is signal amplification. See generally Sylvanen et al., Genomics 8:684-692 (1990); U.S. Pat. Nos. 5,846,710 and 5,888,819; Pastinen et al., Genomics Res. 7(6):606-614 (1997); all of which are expressly incorporated herein by reference. See generally U.S. Pat. Nos. 5,185,243, 5,679,524 and 5,573,907; EP 0 320 308 B1; EP 0 336 731 B1; EP 0 439 182 B1; WO 90/01069; WO 89/12696; WO 97/31256; and WO 89/09835, and U.S. Ser. Nos. 60/078,102 and 60/073,011, all of which are incorporated by reference.

In some aspects, examples of PCR that can be used in the invention include, but is not limited to, nucleic acid sequence-based amplification (NASBA), strand displacement amplification (SDA), multiple displacement amplification (MDA), Q-beta replicase amplification, and loop-mediated isothermal amplification are used for amplification.

In some aspects, the amplification method may be specific for a certain nucleic acid such as a specific gene or a fragment thereof, or may be universal such that all or a specific type of a nucleic acid, such as mRNA, is amplified universally. In some aspect, the skilled person may design oligonucleotide primers which specifically hybridize to the nucleic acid of interest and use these primers in a PCR experiment.

In some aspects, the amplification method may use a master mix. In some aspects, the master mix may be a premixed, ready-to-use solution that may contain DNA polymerase, dNTPs, MgCl₂ and reaction buffers at optimal concentrations for efficient amplification of DNA templates. In some aspects, the master mix may contain DNA polymerase. In some aspects, the DNA polymerase may be a Taq DNA polymerase. In some aspects, the Taq DNA polymerase may be modified. In some aspects, the DNA polymerase may display no enzymatic activity at ambient temperature. In some aspects, the DNA polymerase may not form misprimed products and/or primer dimers prior to the first denaturation step. In some aspects, the DNA polymerase may be activated during the first denaturation step. In some aspects, the DNA polymerase may be activated after about 1 second to about 15 minutes during the first denaturation step. In some aspects, the DNA polymerase may be activated after about 5, 10, 15, 20, 25, 30, 40, 50, 60, 90, 120, 150, 180, 210, 240, 300, 350, 400, 500, 600, 700, 800, or about 900 seconds. In some aspects, the DNA polymerase may have 5′-3′ polymerase activity. In some aspects, the DNA polymerase may have 5′-3′ endonuclease activity. In some aspects, the DNA polymerase may have 3′-5′ exonuclease activity. In some aspects, the DNA polymerase may not have 3′-5′ exonuclease activity. In some aspects, the DNA polymerase may have 5′-3′ polymerase activity and 5′-3′ endonuclease activity, but no 3′-5′ exonuclease activity. In some aspects, the DNA polymerase may have an error rate of approximately 1 error per 2.2×10⁵ nucleotides incorporated. In some aspects, the DNA polymerase may have an error rate of in the range of about 1 error per 2.2×10² to about 1 error per 2.2×10¹⁵ nucleotides incorporated.

In some aspects, the master mix may contain one or more dyes. In some aspects, the one or more dyes may be fluorescent. In some aspects, the one or more dyes may be a reference dye. In some aspects, the reference dye may be a passive reference dye. In some aspects, the reference dye may be a ROX reference dye. In some aspects, the master mix may contain MgCl₂. In some aspects, the master mix may not contain MgCl₂. In some aspects, the master mix may contain dNTPs. In some aspects, the master mix may contain stabilizers. In some aspects, the master mix may be free of contaminating DNase and/or RNase. In some aspects, the master mix may be added at a final concentration in a range of about 0.1 mM to about 50 mM. In some embodiments the master mix may be added at a concentration of about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100 5, 10, 15, 20, 25, 30, 35, 40, 45, or about 50 mM. In some aspects, the master mix may be added at a final concentration greater than about 50 mM. In some aspects, the master mix may be added at a final concentration less than about 0.1 mM.

Detection

The act of testing a sample for a pathogen or a change in the level of a pathogen, is a “detection” even if the microorganism is determined to be not present or below the level of sensitivity. Detection may be a quantitative, semi-quantitative or non-quantitative observation and may be based on a comparison with one or more control samples. Detection may be applied to any sample wherein the presence or absence of the pathogen is to be assessed. in some aspects, and without limitation, the step of detecting a pathogen may comprise using PCR, real-time PCR, lectins, simple diffusion, lateral diffusion, immunological detection, lateral flow, or flow through methods to detect the presence of the pathogen in a culture. By way of illustration and not limitation, in particular embodiments possible detecting methods include or use the subject matter disclosed in any of U.S. Pat. Nos. 6,483,303; 6,597,176; 6,607,922; 6,927,570; and US7323139.

In some aspects, pathogens may be detected individually. In some aspects, multiple pathogens may be detected simultaneously. In some aspects, pathogen detection may be by a detection assay such as multiplex PCR, multiplex ELISA, DNA microarray, protein microarray or bead based assays such as a Luminex assay. In some aspects, luminex assays may use microspheres.

In some aspects, the present invention is to any detection method that allows for detecting one or more pathogens. In some aspects, the present primers, oligonucleotides probes, methods, materials, compositions, kits, and components allow for the detecting of one or more pathogens. In some aspects, the one or more pathogens may be alive. In some aspects, the one or more pathogens may be dead. In some aspects, the one or more pathogens may be alive and/or dead. In some aspects, alive pathogens may be detected to avoid high false positive results.

In some aspects, in the context of the present invention is any method that allows for detection and/or identification of a specific nucleic acid or a polypeptide, wherein the term “detection” also comprises the quantitative determination of a nucleic acid. In some aspects, the detection and/or identification may be based on specific amplification, for example, by the amplification of a specific DNA fragment using oligonucleotide primers specific for said DNA fragment in the polymerase chain reaction (PCR). In some aspects, the detection and/or identification may be based immunoassays.

In some aspects, detection may be a quantitative, semi-quantitative or non-quantitative observation and may be based on a comparison with one or more control samples. In some embodiments and without limitation, the step of detecting a microorganism comprises using PCR, real-time PCR, lectins, multiplex PCR, PCR methods disclosed herein, simple diffusion, lateral diffusion, immunological detection, lateral flow, or flow through methods to detect the presence of the microorganism in a culture. By way of illustration and not limitation, in particular embodiments possible detecting methods include or use the subject matter disclosed in any of U.S. Pat. Nos. 6,483,303; 6,597,176; 6,607,922; 6,927,570; and 7,323,139.

The skilled person is well aware of how to design oligonucleotide primers which specifically hybridize to the nucleic acid of interest. In some aspects, the detection and/or identification may also be achieved without amplification, for example, by sequencing the nucleic acid to be analyzed or by sequence specific hybridization, for example, in the context of a microarray experiment. Sequencing techniques and microarray based analysis are well known procedures in the field. In some aspects, detection after PCR can be performed by, for example, electrophoresis, fluorescent probe method, capillary electrophoresis method, or quantitative PCR method.

In some aspects, the detection described herein comprises a detection capacity that meets and/or exceeds regulatory requirements. In some aspects, the invention described herein may detect at least 0.5 colony forming unit (CFU) in a standard overnight culture. In some aspects, a standard overnight culture can be 25 g food+225 ml media. In some aspects, the invention described herein may have a sensitivity threshold of detecting at least about 0.05 CFU/25 g. In some aspects, the invention described herein may have a sensitivity threshold of detecting at least about 0.005 CFU/25 g. In some aspects, the invention described herein may have a sensitivity threshold of detecting at least about 0.0005, 0.005, 0.05, 0.1, 0.2, 0.3, 0.4, 0.48, 0.5, 0.6, 0.7, 0.8, 0.9, 1, 1.1, 1.2, 1.5, 2, 2.5, 3 or at least 5 CFU/25 g. In some aspects, the invention described herein may have a sensitivity threshold of detecting less than about 0.05 CFU/25 g. In some aspects, the invention described herein may have a sensitivity threshold of detecting less than about 0.005 CFU/25 g.

Sequencing

Any high-throughput technique for sequencing nucleic acids can be used in the method of the invention. In some aspects, DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing, nanopore sequencing. Sequencing of the separated molecules has more recently been demonstrated by sequential or single extension reactions using polymerases or ligases as well as by single or sequential differential hybridizations with libraries of probes.

Detecting

As described herein, in some embodiments, the kits and method described herein utilize detection of target sequences by detection of amplicons. In some embodiments, either direct or indirect detection of amplicon can be performed. In some embodiments, direct detection involves the incorporation of a label into the amplicon via, e.g., a labeled primer. In some embodiments, indirect detection involves incorporation of a label into, e.g., a hybridization probe. In some embodiments, for direct detection, the label(s) may be incorporated in at least four ways: (1) the primers comprise the label(s), for example attached to the base, a ribose, a phosphate, or to analogous structures in a nucleic acid analog; (2) modified nucleosides that are modified at either the base or the ribose (or to analogous structures in a nucleic acid analog) with the label(s); these label-modified nucleosides are then converted to the triphosphate form and are incorporated into the newly synthesized strand by a polymerase; (3) modified nucleotides are used that comprise a functional group that can be used to add a detectable label; or (4) modified primers are used that comprise a functional group that can be used to add a detectable label. In some embodiments, any of these methods result in a newly synthesized strand that comprises labels that can be directly detected.

In some embodiment, for indirect detection, the label may be incorporated into a hybridization probe using methods well known to one of skill in the art. In some embodiments, the label can be incorporated by attaching the label to a base, ribose, phosphate, or to analogous structures in a nucleic acid analog, or by synthesizing the hybridization probe using a modified nucleoside. In some embodiments, a modified strand of the amplicon or the hybridization probe can include a detection label. By “detection label” or “detectable label” herein is meant a moiety that allows detection. This may be a primary label or a secondary label.

In some embodiments, the detection label is a primary label. In general, labels fall into three classes: a) isotopic labels, which may be radioactive or heavy isotopes; b) magnetic, electrical, thermal labels; and c) colored or luminescent dyes. In some embodiments, labels can also include enzymes (horseradish peroxidase, etc.) and magnetic particles. In some embodiments, labels include chromophores or phosphors but in some embodiments are fluorescent dyes. In some embodiments, suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue™, Texas Red, alexa dyes, phycoerythrin, bodipy, and others described in the 6th Edition of the Molecular Probes Handbook by Richard P. Haugland, hereby expressly incorporated by reference.

In some embodiments, a secondary detectable label is used, for example, a secondary label can bind or react with a primary label for detection, or may allow the separation of the compound comprising the secondary label from unlabeled materials, etc. In some embodiments, secondary labels include, but are not limited to, one of a binding partner pair; chemically modifiable moieties; nuclease inhibitors, etc. In some embodiments, the secondary label may comprise a binding partner pair, for example, the label may be a hapten or antigen, which will bind its binding partner. In some embodiments, the binding partner can be attached to a solid support to allow separation of extended and non-extended primers, for example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. In some embodiments, nucleic acid-nucleic acid binding proteins pairs are also useful. In some embodiments, the binding partner pair comprises biotin or imino-biotin and streptavidin Imino-biotin is particularly preferred as imino-biotin disassociates from streptavidin in pH 4.0 buffer while biotin requires harsh denaturants (e.g. 6 M guanidinium HCl, pH 1.5 or 90% formamide at 95° C.). In some embodiment, the binding partner pair comprises a primary detection label and an antibody that will specifically bind to the primary detection label.

Probes

In some aspects, detection can be performed in a PCR mixture by using fluorescently labeled probes, each one corresponding to a unique DNA sequence, which when amplified by a DNA polymerase, emit a fluorescence signal at its specified spectral wavelength. In some aspects, the spectral frequency discrimination between different fluorophores, or reporters, attached to each probe sequence enables detection of amplicon sequences, one for each fluorescent color that can be identified.

In some aspects, in the detection method of the present invention, a process comprising the steps of mixing the above DNA and/or RNA and one or more primers specific to the target pathogens to be detected, to perform multiplex PCR, is indispensable. In some aspects, the primers used are specific to target pathogens to be detected and/or an internal control. In some aspects, the primers have similar melting temperature that do not mutually produce primer dimmer, or wherein their identification bands do not interfere or overlap each other. In some aspects, primer sets include, for example, primer set specific for pathogenic Salmonella Invasion Gene (Inv), SEQ ID NOs 17 and 18, 25 and 26; LSP IAD, SEQ ID NOs 11 and 12; LG IAD, SEQ ID NOs 27 and 28; Listeria monocytogenes gene Listeriolysin 0 (HlyA), SEQ ID NOs 9 and 10; and Shiga Toxin-Producing Escherichia coli genes Shiga toxin 1 (stx1), SEQ ID NOs 21 and 22, 23 and 24; Shiga toxin 2 (stx2), SEQ ID NOs 15 and 16; encoding intimin (eae), SEQ ID NOs 13 and 14; and an internal control, SEQ ID NOs 19 and 20 can be exemplified respectively, and these can be used in combinations.

In some aspects, amplification is performed with the addition of labeled and or unlabeled probes. In some aspects, oligonucleotide probes include, for example, oligonucleotide probes specific for pathogenic Salmonella Invasion Gene (Inv), SEQ ID NOs 6; Listeria monocytogenes gene Listeriolysin 0 (HlyA), SEQ ID NOs 1; LSP IAD, SEQ ID NOs 2; LG IAD SEQ ID NOs 7; Shiga Toxin-Producing Escherichia coli genes Shiga toxin 1 (stx1), SEQ ID NOs 4; Shiga toxin 2 (stx2), SEQ ID NOs 5; encoding intimin (eaeA), SEQ ID NOs 3; and an internal control, SEQ ID NO 9 can be exemplified respectively, and these can be used in combinations.

In some aspects, amplification is performed with the addition of probes and primer sequences. For example, as for primer set and probe specific for pathogenic Salmonella Invasion Gene A (InvA), Primer SEQ ID NOS 9 and 10, Probe SEQ ID NOS 15; Listeria monocytogenes gene Listeriolysin 0 (HlyA), Primer SEQ ID NOS 1 and 2, Probe SEQ ID NOS 11; and Shiga Toxin-Producing Escherichia coli genes Shiga toxin 1 (stx1), Primer SEQ ID NOS 5 and 6, Probe SEQ ID NOS 13; Shiga toxin 2 (stx2), Primer SEQ ID NOS 7 and 8, Probe SEQ ID NOS 14; encoding intimin (eaeA), Primer SEQ ID NOS 2 and 3, Probe SEQ ID NOS 12; and an internal control, SEQ ID NO 18 can be exemplified respectively, and these can be used in combinations.

In some embodiments the quantity of primer per reaction may be at least about 0.001 nmol. In some embodiments the quantity of primer per reaction may be at least about 0.001, 0.01, 0.1, 0.3, 1, 3, 4, 10, 30, 40, 60, 100, 250, 300, 350, 400, 500, 750, 1000, 1500, 2500, or at least about 5000 nmol.

In some embodiments the quantity of probe per reaction may be at least about 0.001 nmol. In some embodiments the quantity of probe per reaction may be at least about 0.001 0.1, 0.3, 1, 3, 4, 10, 30, 40, 60, 100, 250, 300, 350, 400, 500, 750, 1000, 1500, 2500, or at least about 5000 nmol.

In some embodiments, the quantity of internal control may be at least 25 copies of internal control reference gene per reaction. In some embodiments the quantity of internal control may be at least about 25, 500, 1000, 2000, 3000, 5000, 10000, 15000, 20000, 25000, 30000, 35000, 40000, 50000, 80000, 100000, 150000, or at least about 200,000, or at least about 300,000 copies of internal control reference gene per reaction.

In some aspects, the oligonucleotide probes are TaqMan probes. TaqMan probes are hydrolysis probes that are designed to increase the specificity of PCR assays. A standard TaqMan probe comprises a fluorophore covalently attached to the 5′-end of an oligonucleotide probe and a quencher at the 3′-end. In some aspects, during PCR amplification, the primers and fluorescently tagged probes anneal to the DNA template, and as the polymerase extends the primer sequences, the fluorescent label is cleaved from the probe strand, thereby increasing its distance from the quencher and allowing the fluorophore to emit fluorescence with greater intensity.

In some aspects, several different fluorophores (e.g. 6-carboxyfluorescein, acronym: FAM, or tetrachlorofluorescein, acronym: TET, or 6-carboxy-4′,5′-dichloro-2′,7′-dimethoxyfluorescein, acronym: JOE) and quenchers (e.g. tetramethylrhodamine, acronym: TAMRA or Black Hole Quencher™ 1 (BHQ1 Acronym: BHQ1) are available. Several fluorophore-quencher pairs are described in the art. See, e.g. Pesce et al, editors, Fluorescence Spectroscopy, Marcel Dekker, New York, (1971); White et al, Fluorescence Analysis: A Practical Approach, Marcel Dekker, New York, (1970); and the like. The literature also includes references providing exhaustive lists of fluorescent and non-fluorescent molecules and their relevant optical properties, e.g. Berlman, Handbook of Fluorescence Sprectra of Aromatic Molecules, 2nd Edition, Academic Press, New York, (1971), herein incorporated by reference. Further, there is extensive guidance in the literature for derivatizing reporter and quencher molecules for covalent attachment via common reactive groups that can be added to an oligonucleotide. See, e.g. U.S. Pat. Nos. 3,996,345; and 4,351,760. Exemplary fluorophore-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl-6-naphthalene sulfonate. Other dyes include 3-phenyl-7-isocyanatocoumarin, acridines, such as 9-isothiocyanatoacridine and acridine orange; N-(p-(2-benzoxazolyl)phenyl)maleimide; benzoxadiazoles, stilbenes, pyrenes, and the like. In some aspects, fluorophore and quencher molecules are selected from fluorescein and rhodamine dyes. These dyes and appropriate linking methodologies for attachment to oligonucleotides are known in the art. See, e.g. Marshall, Histochemical J. 7: 299-303 (1975); and U.S. Pat. No. 5,188,934, herein incorporated by reference.

In some aspects, multiplex PCR may be possible using non TaqMan probe reporters such as intercalating dyes by encoding intensity levels to distinguish concentration limited primer pairs alone. In some aspects, intercalating dyes, bind to double-stranded DNA sequences, and an increase in DNA product during PCR therefore leads to an increase in fluorescence intensity. In some aspects, intercalating dyes include but not limited to SYBR or PicoGreen which bind to amplified double stranded DNA. In some aspects, detection by intercalating dyes is performed by adding multiple unique primer pairs at different limiting concentrations to yield varying end point fluorescence intensities.

In some aspects, the fluorescences emitted are detected (e.g., via digital filters) and identified, and the DNA sequences corresponding to the emitted fluorescences may be similarly identified based on their correspondence.

Detecting Non-Amplified Nucleic Acid

In some embodiments, the detecting step can include lysing microorganisms in the sample, hybridizing a nucleic acid probe to a target nucleic acid sequence of the target microorganism to form a probe/target complex, wherein the probe includes a label that is stabilized by the complex, selectively degrading the label present in unhybridized probe, and detecting the presence or amount of stabilized label as a measure of the presence or amount of the target nucleic acid sequence in the sample. In some embodiments, the probe may be labeled with an acridinium ester. In some embodiments, the probe may hybridize to ribosomal RNA of the target microorganism.

In some embodiments, pathogens can be detected using, for example, a hybridization protection assay (HPA). In some embodiments, pathogens can be lysed to release nucleic acid, and an oligonucleotide probe can be hybridized to a target nucleic acid sequence of the target microorganism to form a probe/target complex wherein the probe is detected.

In some embodiments, nucleic acid analogs can be modified at the base moiety, sugar moiety, or phosphate backbone to improve, for example, stability, hybridization, or solubility of a nucleic acid. Modifications at the base moiety include substitution of deoxyuridine for deoxythymidine, and 5-methyl-2′-deoxycytidine and 5-bromo-2′-deoxycytidine for deoxycytidine. In some embodiments, examples of nucleobases that can be substituted for a natural base include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808, herein incorporated by reference.

In some embodiments, modifications of the sugar moiety can include modification of the 2′ hydroxyl of the ribose sugar to form 2′-O-methyl or 2′-O-allyl sugars. In some embodiments, the deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone (e.g., an aminoethylglycine backbone) and the four bases are retained. See, for example, Summerton and Weller (1997) Antisense Nucleic Acid Drug Dev. 7:187-195; and Hyrup et al. (1996) Bioorgan. Med. Chem. 4:5-23, all of which are hereby incorporated by reference. In some embodiments, the deoxyphosphate backbone can be replaced with, for example, a phosphorothioate or phosphorodithioate backbone, a phosphoroamidite, or an alkyl phosphotriester backbone. See, for example, U.S. Pat. Nos. 4,469,863, 5,235,033, 5,750,666, and 5,596,086 for methods of preparing oligonucleotides with modified backbones. In some embodiments, oligonucleotide probe can hybridize with any portion of a nucleic acid from the target microorganism, for example, an oligonucleotide can hybridize with a nucleic acid encoding a cell-wall protein or an internal cell component, such as a membrane protein, transport protein, or enzyme. In some embodiments, the oligonucleotide hybridizes with ribosomal RNA (rRNA) or a mRNA of a target microorganism. See, for example, U.S. Pat. No. 4,851,330, which is hereby incorporated by reference. For example, the oligonucleotide can hybridize with a 16S, 23S, or 5S rRNA. In some embodiments, hybridization to rRNA can increase the sensitivity of the assay as most microorganisms contain thousands of copies of each rRNA.

In some embodiments, the oligonucleotide probe is labeled with a molecule that is stabilized by the probe/target. In some embodiments, oligonucleotide probes can be between 10 and 75 (e.g., 10-14, 15-30, 25-50, 30-45, 33-40, 20-30, 31-40, 41-50, or 51-75) nucleotides in length. In some embodiments, the oligonucleotide need not be 100% complementary to that of its target nucleic acid in order for hybridization to occur. In some embodiments, the oligonucleotide has at least 80% (e.g., at least 85%, 90%, 95%, 99%, or 100%) sequence identity to the complement of its target sequence. In some embodiments, hybridization of the oligonucleotide to its target can be detected based on the chemiluminescence observed after adjusting the pH to mildly alkaline conditions. In some embodiments, if hybridization occurs, chemiluminescence will be observed. In some embodiments, if hybridization does not occur, the ester bond of the AE molecule will be hydrolyzed and chemiluminescence will not be observed or will be measurably reduced.

Methods for synthesizing oligonucleotides are known.

In some embodiments, the presence, absence, or amount of unmodified label can be assessed using a luminometer (e.g., LEADER® luminometer from Gen-Probe Incorporated, San Diego, Calif. or the BacLite3 luminometer from 3M, St. Paul, Minn., or the LUMIstar Galaxy luminometer from BMG, Durham, N.C.). Luminometers such as the BacLite3 luminometer and LUMIstar Galaxy luminometer have reagent dispensing capability and temperature control are particularly useful for automating the methods disclosed herein. Such luminometers can be programmed to dispense, in a predetermined order, reagents for lysing, hybridization, and detection, and allow for incubation. Automated reagent dispensing minimizes contamination issues encountered within a moist environment such as a water bath in addition to enhancing the user friendliness of the test system. It is understood that the present method is not limited by the device used to detect the label on the oligonucleotide probe.

Primer and Probe Design

In some aspects, literature and Blast searches may be performed to identify sets of genes with the potential to uniquely identify pathogenic target organisms in the context of a 5-color multiplex TaqMan-based PCR reaction. In some aspects, the genes chosen from these searches may be: Salmonella Invasion Gene A (InvA), Listeria monocytogenes gene Listeriolysin 0 (HlyA), and Shiga Toxin-Producing Escherichia coli genes Shiga toxin 1 (stx1), Shiga toxin 2 (stx2), and encoding intimin (eaeA).

In some embodiments, sets of multiplex PCR primers and TaqMan probes may be designed using commercial software and genomic DNA sequences. In some aspects, specificity of resulting sequences may be assessed in silico against the nr database using Blast. In some aspects, optimal PCR conditions may be identified for each of the multiplex sets. In some aspects, selection of a final set may be done in a step-wise manner. In some aspects, compatibility, sensitivity, and specificity may be initially assessed using purified genomic DNA from target organisms and with non-target bacteria DNA. In some aspects, sets may be tested using DNA prepared from bacteria cultured in the presence of various food matrices. Nucleic Acid Reagents: Primers and Probes

In some embodiments, the kits and method disclosed herein use nucleic acid reagents, e.g., oligonucleotides, e.g., amplification primers and hybridization probes, for detection of the signature sequences. In some aspects, exemplary primers and probes are disclosed herein, e.g., in Table 1, and in some embodiments, the claimed kits and methods include the primers and probes disclosed in Tables 1. In some embodiments, the invention also include kits and methods using variant versions of the primers and probes disclosed herein, e.g., oligonucleotides that are shorter or longer or have at least about 80%, 81%, 82%, 83%, 84%, 85%, 86%, 88%, 89, 90%, 91%, 92%, 93%, 94%, 95%, 96%, 97%, 98%, or at least about 99% sequence identity, as long as the oligonucleotide accomplishes that same function, e.g., functions in the assay for the detection of the signature sequences. In some embodiments, the kit can comprise probes and or primers comprising about 80%, 81%, 82%, 83%, 84%, 85%, 86%, 88%, 89, 90%, 91%, 92%, 93%, 94%, 95%, 96%, 97%, 98%, or at least about 99% sequence identity to the probes and primers of Table 1. In some aspects, a primer comprises at least 15 contiguous bases that are at least 70%, 80%, 90%, 100% homologous with a sequence listed in Table 1.

In some embodiments, the length of a nucleic acid reagent, e.g., a primer or hybridization probe or oligonucleotide probe, will vary depending on the application. In some embodiments, the total length can be from about 5 to 80 nucleobases in length. In some embodiments, the primers, oligonucleotide probe and hybridization probes used in accordance with this invention may comprise from about 8 to about 80 nucleobases (i.e. from about 8 to about 80 linked nucleosides). One of ordinary skill in the art will appreciate that the invention embodies oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length. In some embodiments, the oligonucleotides are greater than 80 nucleobases in length.

In some embodiments, the kits include nucleic acid reagents that are sets of oligonucleotides for each target sequence to be detected. Each set has PCR primers, oligonucleotide probe and or hybridization probes for each target sequence. Exemplary embodiments include the PCR primers and oligonucleotide probes disclosed in Tables 1. In some embodiments the kit includes each of the PCR primers and oligonucleotide probes listed for the respective pathogen. In some embodiments, the kit includes a subset of the disclosed primer and probes. In some embodiments, the kit includes at least 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 40, or at least 50 of the primer pairs. In some embodiments, the kit includes at least 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 40, or at least 50 oligonucleotide probes.

In some embodiments, the kits includes reagents for detection of less than all pathogens, e.g., for detection of at least 1, 2, 3, 4, 5, 6, 7, 8, 9, 10, 11, 12, 13, 14, 15, 16, 17, 18, 19, or at least 20 of the pathogens. In some embodiments, TaqMan probes, can be detected by methods known in the art.

Internal Controls

When testing a biological sample for contamination by a pathogen or confirming that a sample is free from a pathogen, a problem may exist with interpreting a negative result. Without appropriate controls, it may not be possible to determine whether an absence of the contaminating pathogen being detected is a result of the failure of the assay, or as a result of the absence of any contaminating pathogens in the sample. If the negative result can be attributed to the former reason, the failure of the assay could have occurred at any stage. For example, in a nucleic acid assay, the failure may have occurred during nucleic acid extraction, handling, amplification or detection steps. Generally, for example and not to be limiting, four controls may be used in PCR based methods for the detection of nucleic acids. The first control may be an internal positive control for the nucleic acid extraction step. The second control may be for the detection of the PCR products. The third control may be for the amplification step. Finally, the fourth control may be a no template control to detect contamination during the assay.

In some aspects, amplification may be performed with an internal control. In some aspects, the internal control may be a negative control. In some aspects, the internal control may be a positive control.

In some aspects, the internal control may be a polynucleotide or oligonucleotide. In some aspects, the internal control may be an exogenous sequence. In some aspects, the internal control may be used as a universal internal control as it comprises unique primer and probe sites and does not exhibit homology with any known nucleic acid sequences that may interfere with this assay, i.e. does not anneal with known nucleic acid sequences during conventional PCR techniques.

In some aspects, the internal control may be DNA and or RNA molecules of natural or synthetic origin which may be single-stranded or double-stranded, and represent the sense or antisense strand. In some aspects, the internal control may be a sequence chosen as required in an amplification reaction. In some aspects, the internal control may be a sequence selected from, e.g., sequences that are suitable to detect and/or distinguish pathogenic material such as viruses, bacteria, fungi, parasites such as Plasmodium falciparum, ticks, E. coli etc. In some aspects, the internal control may contain known nucleotide analogs or modified backbone residues or linkages, and any substrate that can be incorporated into a polymer by DNA or RNA polymerase. Examples of such analogs include phospborothioates, phosphoramidates, methyl phosphonates, chiral-methyl phosphonates, 2-O-methyl ribonucleotides, peptide-nucleic acids (PNAs), and the like. In some aspects, the internal control may be isolated. In some aspects, the internal control may be substantially isolated or purified from the genomic DNA or RNA of the species from which the nucleic acid molecule was obtained.

In some aspects, the internal control may be readily prepared by conventional methods known in the art, for example, directly synthesizing the nucleic acid sequence using methods and equipment known in the art such as automated oligonucleotide synthesizers, PCR technology, recombinant DNA techniques, and the like. WO 2003075837 A2, WO 2012114312 A2 and WO 2012114312 A2 are herein incorporated by reference.

In some aspects, an internal control probe may be used to detect the presence and or absence of the internal control. In some aspects, the internal control probe may be an internal oligonucleotide probe. In some aspects, the internal oligonucleotide probe may be labeled at the 5′ end with an energy transfer donor fluorophore and labeled at the 3′ ends with an energy transfer acceptor fluorophore. In some aspects, the internal oligonucleotide probe specifically anneals between the forward and reverse primers of a target sequence. In some aspects, the internal oligonucleotide probe may be cleaved by the 5′ end during PCR amplification and the reporter molecule may then separate from the quencher molecule to generate a sequence specific signal. In some aspect, with each amplification cycle, additional reporter molecules may be separated from the quencher molecules. In some aspects, the intensity of a signal, such as fluorescence, may be monitored before, during, or after PCR amplification or a combination thereof.

In some aspects, the internal control may be used to distinguish a true negative result from a false negative result. As used herein, a “true negative” result correctly indicates that a sample lacks a target nucleic acid sequence. A “false negative” result incorrectly indicates the absence of a target nucleic acid sequence which may result from PCR inhibitors present in the sample or technical error.

In some embodiments, the detection methods disclose herein, may detect the presence or absence of one or more pathogens in a sample with an accurately in the range of between at least 1% to at least 99.9%. In some embodiments, the detection methods disclose herein, may detect the presence and/or absence of one or more pathogens in a sample with an accurately of at least 10%, 15%, 20%, 25%, 30%, 35%, 40%, 45%, 50%, 55%, 60%, 65%, 70%, 75%, 80%, 85%, 90%, 95%, 96%, 97%, 98% or at least 99%. In some embodiments, the detection methods disclose herein, may detect the presence and/or absence of one or more pathogens in 1/5, 2/5, 3/5, 4/5, or 5/5 replicates. In some embodiments, the detection methods disclose herein, may detect the presence and/or absence of one or more pathogens in 1/20, 2/20, 3/20, 4/20, 5/20, 6/20, 7/20, 8/20, 9/20, 10/20, 11/20, 12/20, 13/20, 14/20, 15/20, 16/20, 17/20, 18/20, 19/20, or 20/20 replicates. In some embodiments, the detection methods disclose herein, may detect the presence and/or absence of one or more pathogens in a range of between at least 10% and 99.9% of replicates. In some embodiments, the detection methods disclose herein, may detect the presence and/or absence of one or more pathogens in at least 10%, 15%, 20%, 25%, 30%, 35%, 40%, 45%, 50%, 55%, 60%, 65%, 70%, 75%, 80%, 85%, 90%, 95%, 96%, 97%, 98% or at least 99% of replicates.

### Effect

In some embodiments, the kits, devices and method disclosed herein overcome the problem of detecting multiple organisms that are considered incompatible with simultaneous enrichment. In some embodiments, the enrichment media and/or enrichment methods disclosed herein overcome the problem of detecting multiple organisms that are considered incompatible with simultaneous enrichment. In some embodiments, the lysis buffers and/or lysis methods disclosed herein overcome the problem of detecting multiple organisms that are considered incompatible with simultaneous enrichment. In some embodiments, the enrichment methods and/or enrichment media and lysis buffers and/or lysis procedures disclosed herein overcomes the problem of detecting multiple organisms that are considered incompatible with simultaneous enrichment.

In some embodiments, the enrichment media, enrichment procedures, lysis buffer, and lysis procedures may confer a synergistic effect on the sensitivity of the detection methods disclosed herein. In some embodiments, the enrichment media, enrichment procedures, lysis buffers, and lysis procedures disclosed herein may increase pathogen detection efficiency. In some embodiments, the enrichment media, enrichment procedures, lysis buffer, and lysis procedures may confer an additive effect on the sensitivity of the detection methods disclosed herein. In some embodiment, the sensitivity of the detection methods disclosed herein may increase when the enrichment media/procedure, lysis buffer/procedure, and assays disclosed herein are used in concert.

In some embodiments, the enrichment media, enrichment procedures, lysis buffer, and lysis procedures may confer a synergistic effect on the pathogen detection time of the detection methods disclosed herein. In some embodiments, the enrichment media, enrichment procedures, lysis buffers, and lysis procedures disclosed herein may decrease the pathogen detection time synergistically. In some embodiments, the enrichment media, enrichment procedures, lysis buffer, and lysis procedures may confer an additive effect on the detection time of the detection methods disclosed herein. In some embodiment, the pathogen detection time of the detection methods disclosed herein may decrease in an additive manner when the enrichment media/procedure, and lysis buffer/procedure, disclosed herein are used in concert.

TABLE 1
Probe Sequences and Primer Sequences.

| SEQ ID | | |
|---|---|---|
| NOs: | Probe Sequence | Sequence Name |
| 1 | ATTGCCAGGTAACGCAAGAAT | LM_HLY-BT_probe |
| 2 | AAGCARGMAKTTCGYTYCACAGGTTT | LSP_IAD-BT_probe |
| 3 | ACGGAAGCCAAAGCGCACT | EC_EAE-BT_probe |
| 4 | ATCTGGATTTAATGTCGCATAGCGT | EC_STX1-BT_Probe |
| 5 | ATTCCATGACAACGGACAGCT | EC_STX2-BT_Probe |
| 6 | AATCGGGCCGCGACTTCT | SE_INV-BT_Probe |
| 7 | AACGCAGTTAAAGTGGATACGAACGCTT | LG_IAD-BT_probe |
| 8 | AACTACATCCTCTCCGCAGCACACT | IC_Probe |
| | Primer Sequence | |
| 9 | AAGCTCATTTCACATCGTCCATCT | LM_HLY-BT_sense |
| 10 | ATCCACCATTCCCAAGCTAAACCT | LM_HLY-BT_antisense |
| 11 | AGTTGGMTTYGGTCGYGTATAAT | LSP_IAD-BT_sense |
| 12 | GAGTTTTTCATAAAGCACCGTCTTT | LSP_IAD-BT_antisense |
| 13 | AGGCGGCCAGATTCAGCATAGT | EC_EAE-BT_sense |
| 14 | AGCTACCACCTTGCACATAAGCT | EC_EAE-BT_antisense |
| 15 | ACAGTGCCCGGTGTGACAACT | EC_STX2-BT_sense |
| 16 | AGACACGTTGCAGAGTGGTATAACT | EC_STX2-BT_antisense |
| 17 | ACGGGCATACCATCCAGAGAAT | SE_INV_BT_sense |
| 18 | ACACCGTGGTCCAGTTTATCGTT | SE_INV_BT_antisense |
| 19 | ATGGCGGGACTATTCTGAATGAGT | IC_sense |
| 20 | ACATCTCGCTGCTGTCTTTCTTCT | IC_antisense |
| 21 | AATGCAGATAAATCGCCATTCGTTGAT | EC_STX1-BT_sense |
| 22 | AACATCGCTCTTGCCACAGACTGT | EC_STX1-BT_antisense |
| 23 | ATCGCCATTCGTTGACTACTTCT | EC_STX1-BT_sense |
| 24 | AACATCGCTCTTGCCACAGACTT | EC_STX1-BT_antisense |
| 25 | ACGGGCATACCATCCAGAGAAT | SE_INV_BT_sense |
| 26 | ACACCGTGGTCCAGTTTATCGTT | SE_INV_BT_antisense |
| 27 | ACCCACAAAGCAGAAGCAAAAGT | LG_IAD-BT_sense |
| 28 | ACAGGAACGCCATATTTGACAGT | LG_IAD-BT_antisense |
| 29 | ATTTTGTGGCGGGACTATTCTGAATGAGTAC | Synthetic template of |
| | TACAATCCTCTCCGCAGCACACTGCATGCAC | internal control |
| | CAAGCAAAAAAGATTCAAAGTTAGAGTAGG | |
| | GGAACGGGAACACCGAGAAGAAAGACAGCA | |
| | GCGAGATGGCGCAT | |

TABLE 2
Degenerate nucleotide code:

| | |
|---|---|
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

### EXAMPLES

Method 1

Enrichment procedure: A weight of 25 grams of sample was suspended in 225 ml selective enrichment media and stomached for 30 seconds. The bag was closed and incubated at 37° C. 23 hours+/−1 hour.

TABLE 3
Selective Enrichment Media

| | Amount Per Liter (gram) |
|---|---|
| Media Components | |
| Beef Heart Solids | 0-8.0 |
| Calf Brain Solids | 0-10.0 |
| Calf Brain-Beef Heart Infusion | 0-35 |
| Casein Peptone | 0-16.0 |
| Dextrose | 0-10.0 |
| Dipotassium Phosphate | 0-7.0 |
| Disodium Phosphate | 0-20.0 |
| Enzymatic Digest Of Soy | 0-8.0 |
| Esculin | 5-3.0 |
| Ferric Ammonium Citrate | 0-10 |
| Meat Peptone | 0-8.0 |
| Sodium Chloride | 0-10.0 |
| Pancreatic Digest Of Casein | 0-35.0 |
| Peptic Digest Of Animal Tissue | 0-10.0 |
| Porcine Brain Heart Infusion | 0-12 |
| Potassium Phosphate | 0-5.0 |
| Sodium Pyruvate | 0-4.0 |
| Yeast Extract | 0-14.0 |
| Selective Agents | |
| Acriflavine hydrochloride | 0-1 |
| Cycloheximide | 0-1 |
| Lithium Chloride | 0.0-10 |
| Nalidixic acid | 0-1 |

Lysis Procedure: 150 μl of lysis buffer solution was pipetted into each well of a deep-well block. An enrichment bag was placed in a stomacher for 30 seconds at 130 RPM. 50 μl of supernatant was removed from the enrichment bag and added to each well and lysed at 65° C. for 15 minutes shaking at 1350 RPM.

TABLE 4
Lysis buffer
Lysis Buffer

3-12 mM EDTA
30-120 mM TRIS
2-8% Triton X-100
10-40% Proteinase K
5-30 mg/ml Lysozyme
.1-3 grams/ml 100 μm zirconium lysis beads

Primer quantity per reaction: 10-100 nmol per sequence per target. 1 μl of lysate added to 19 μl PCR reaction. Reactions were run on Quantstudio 5, ABI 7500 Fast. With the following thermocycling conditions: Lid temperature of 105° C.; 20 μl reaction; Step 1: 25.0° C. for 2:00 minutes; Step 2: 53.0° C. for 10:00 minutes; Step 3: 95° C. for 2.0 minutes; Step 4: 95° C. for 10 seconds; Step 5: 58.5° C. for 45 seconds; Plate read step—Go to Step 4, repeat 49 more times.

TABLE 4
Mix

| | |
|---|---|
| 5.0 uL | Multiplex Mix |
| 0.2 uL | Fwd Primer (10-100 uM) STX-1 |
| 0.2 uL | Rev Primer (10-100 uM) STX-1 |
| 0.2 uL | Fwd Primer (10-100 uM) STX-2 |
| 0.2 uL | Rev Primer (10-100 uM) STX-2 |
| 0.2 uL | Fwd Primer (10-100 uM) EAE |
| 0.2 uL | Rev Primer (10-100 uM) EAE |
| 0.2 uL | Fwd Primer (10-100 uM) LG |
| 0.2 uL | Rev Primer (10-100 uM) LG |
| 0.2 uL | Fwd Primer (10-100 uM) LS |
| 0.2 uL | Rev Primer (10-100 uM) LS |
| 0.2 uL | Fwd Primer (10-100 uM) SE |
| 0.2 uL | Rev Primer (10-100 uM) SE |
| 0.2 uL | Fwd Primer (10-100 uM) IC |
| 0.2 uL | Rev Primer (10-100 uM) IC |
| 1.0 uL | Probe (0.5-5.0 uM) STX-1 |
| 1.0 uL | Probe (0.5-5.0 uM) STX-2 |
| 1.0 uL | Probe (0.5-5.0 uM) LG |
| 1.0 uL | Probe (0.5-5.0 uM) EAE |
| 1.0 uL | Probe (0.5-5.0 uM) LS |
| 1.0 uL | Probe (0.5-5.0 uM) SE |
| 1.0 uL | Probe (0.5-5.0 uM) IC |
| 0.3 uL | Internal Control DNA (300 DNA copies) |
| 3.9 uL | PCR-grade water |
| 1.0 uL | Lysate |
| 20.0 uL | Total per reaction |

Fresh Spinach

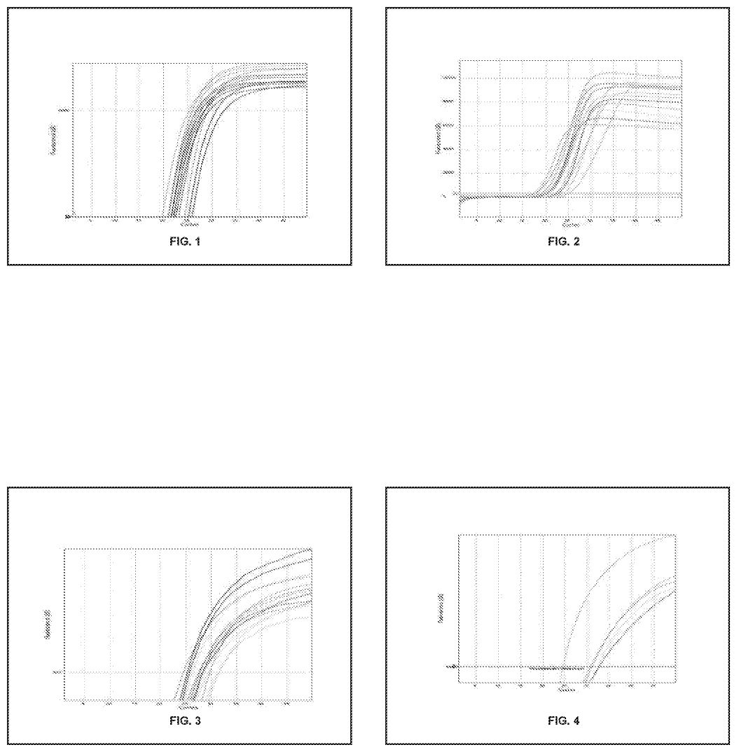

A low-level Inoculation of 20 replicates of 1 CFU/25 g fresh spinach was performed. All 3 target organisms were inoculated, stressed and enriched simultaneously with 0.93 CFU/25 g E. coli O157:H7 (ATCC:43895), 1.28 CFU/25 g S. enterica (ATCC:13076), and 1.25 CFU/25 g L. monocytogenes (ATCC: 13932). All bacteria inoculated into spinach were stored at 4° C. for 48 hours, as per AOAC guidelines. After 48 hours of incubation, samples were enriched at 37° C. for 24 hours in selective enrichment media. An aerobic plate count (APC) was performed on an uninoculated control which indicated 4.4×102 native bacteria per gram. A lysis procedure was performed to maximize recovery and detection of bacterial DNA. Amplification was performed using an Aria Mx Instrument to maximize recovery and detection of bacterial DNA. FIGS. 1-4 show the amplification curves for each of the targets. Primers and probes used are disclosed in Table 1. In FIG. 1—STX-1/STX-2 (2 targets): The method identified 15/20 (75%) replicates as positive for STX-1/STX-2. In FIG. 2—EAE (1 target): The method identified 15/20 (75%) replicates as positive for EAE. In FIG. 3—Salmonella spp. (1 target): The method identified 5/20 (25%) replicates as positive for S. enterica. In FIG. 4—L. monocytogenes (1 target): The method identified 14/20 (70%) replicates as positive for L. monocytogenes. FIG. 5—all targets. FIG. 6—Summary results table notes 25-75% detection of all targets at 1 CFU/25 g was observed, meeting AOAC requirements.

RTE Vegetable (Fresh Spinach) Validation (5 CFU/25 g)

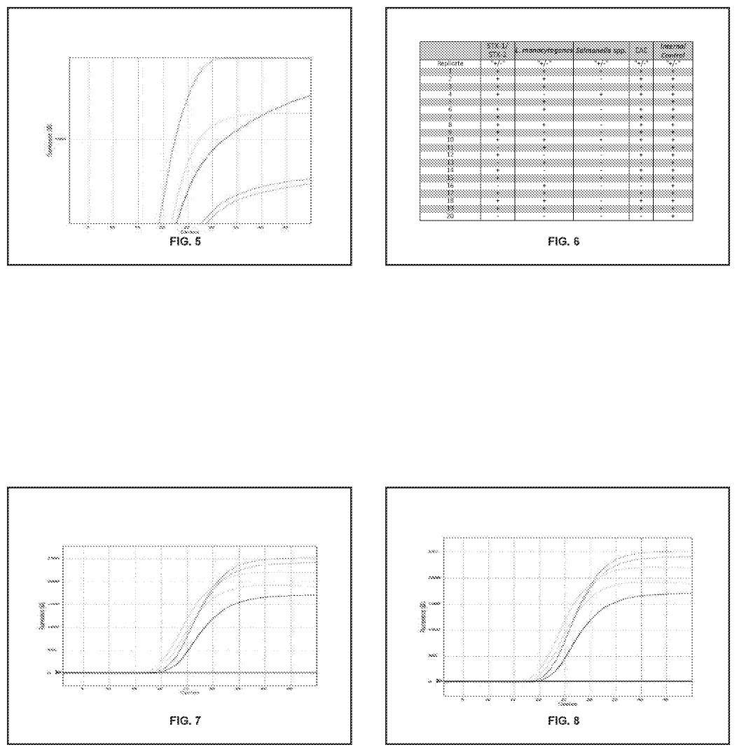

A low-level inoculation of 5 CFU/25 g fresh spinach was performed in 5 replicates. All 3 target organisms were inoculated, stressed and enriched simultaneously with 4.66 CFU/25 g E. coli O157:H7 (ATCC:43895), 6.40 CFU/25 g S. enterica (ATCC:13076), and 6.28 CFU/25 g L. monocytogenes (ATCC: 13932). All bacteria were inoculated into spinach and stored at 4° C. for 48 hours as per AOAC guidelines. After 48 hours of incubation, samples were enriched at 37° C. for 24 hours in the selective enrichment media and an aerobic plate count (APC) performed on uninoculated control indicated 4.4×10² native bacteria per gram. The lysis procedure was performed to maximize recovery and detection of bacterial DNA and amplification of the lysate was performed using an Agilent AriaMx instrument to maximize recovery and detection of bacterial DNA. Primers and probes used are disclosed in Table 1. FIGS. 7-12 show the amplification curves for each target. FIG. 7—Fresh Spinach 5 CFU/25 Gram Inoculation STX-1 and STX-2 Targets. The method detected STX-1 and STX-2 in 5/5 replicates (100% recovery). FIG. 8—Fresh Spinach 5 CFU/25 Gram Inoculation E. coli EAE Target. The method detected E. coli EAE in 5/5 replicates (100% recovery). FIG. 9—Fresh Spinach 5 CFU/25 Gram Inoculation E. coli EAE Target. The method detected E. coli EAE in 5/5 replicates (100% recovery). FIG. 10—Fresh Spinach 5 CFU/25 Gram Inoculation L. monocytogenes Target. The method detected L. monocytogenes in 5/5 replicates (100% recovery). FIG. 11—Fresh Spinach 5 CFU/25 Gram Inoculation S. enterica Target. The method detected S. enterica in 5/5 replicates (100% recovery). FIG. 12—Fresh Spinach 5 CFU/25 Gram inoculation—all targets present. The method detected all targets when present in the same reaction. FIG. 13—Fresh Spinach-5 CFU/25 g—Table of Results. The method detected all targets in 100% of replicates at a 5 CFU inoculation level. The internal control was detected in all replicates.

Raw Ground Beef Validation (1 CFU/25 g)

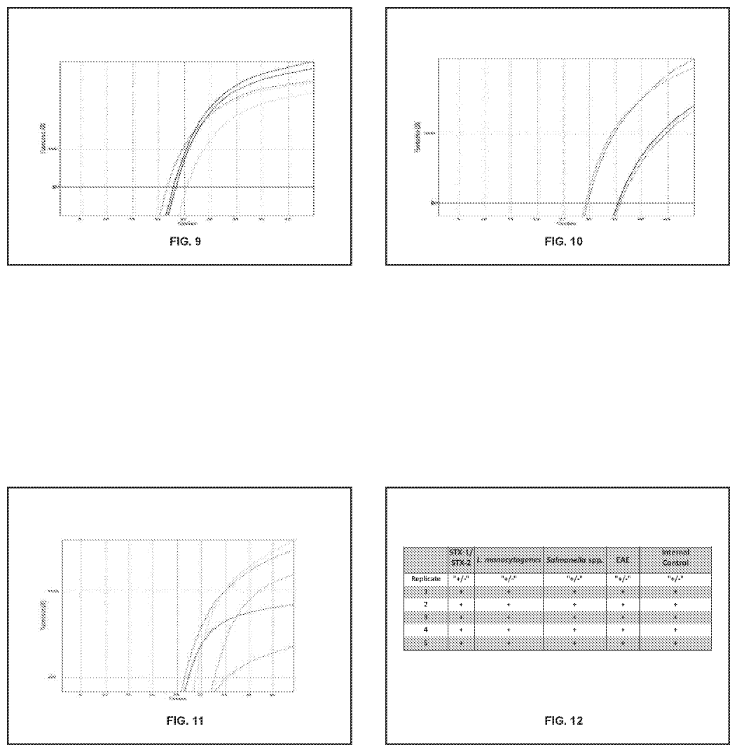

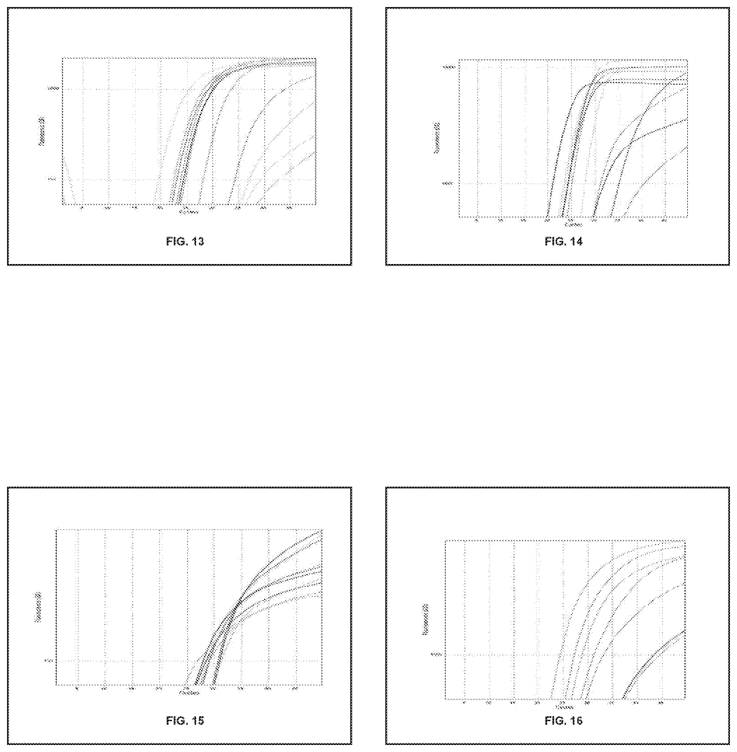

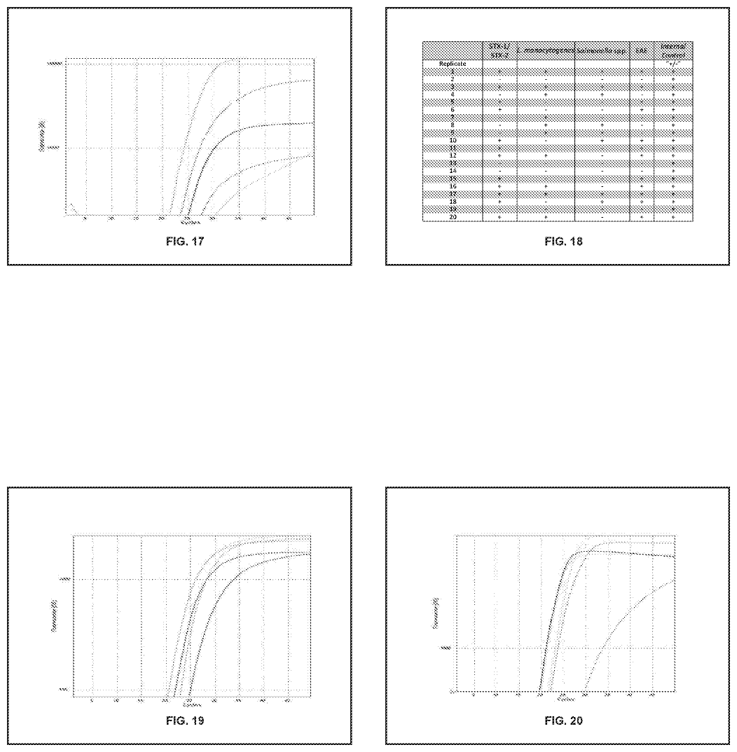

A low-level inoculation of 20 replicates of 1 CFU/25 g ground beef was performed (1 CFU inoculation is AOAC-required lower limit of detection). All 3 target organisms were inoculated, stressed and enriched simultaneously with 1.40 CFU/25 g E. coli O157:H7 (ATCC:43895), 1.09 CFU/25 g S. enterica (ATCC:13076), and 0.86 CFU/25 g L. monocytogenes (ATCC: 13932). All bacteria were inoculated into ground beef and stored at 4° C. for 48 hours as per AOAC guidelines. After 48 hours of incubation, samples were enriched at 37° C. for 24 hours in the selective enrichment media. An aerobic plate count (APC) was performed on an uninoculated control which indicated 5.1×10⁴ native bacteria per gram were present. A lysis procedure was performed to maximize recovery and detection of bacterial DNA. Amplification of the lysate was performed using an Agilent AriaMx Instrument to maximize recovery and detection of bacterial DNA. Primers and probes used are disclosed in Table 1. FIGS. 14-18 show the amplification curves for each target. FIG. 14—Raw Ground Beef 1 CFU/25 Gram Inoculation STX-1 and STX-2 Targets. The method detected STX-1 and STX-2 in 12/20 replicates (60% recovery). FIG. 15—Raw Ground Beef 1 CFU/25 Gram Inoculation E. coli EAE Target. The method detected E. coli EAE in 12/20 replicates (60% recovery). FIG. 16—Raw Ground Beef 1 CFU/25 Gram Inoculation L. monocytogenes Target. The method detected L. monocytogenes in 10/20 replicates (50% recovery). FIG. 17—Raw Ground Beef 1 CFU/25 Gram Inoculation S. enterica Target. The method detected S. enterica in 8/20 replicates (40% recovery). FIG. 18—Raw Ground Beef 1 CFU/25 Gram Inoculation All Targets. The method detected all targets when present in the same reaction. FIG. 19—Raw Ground Beef—1 CFU/25 g—Table of Results. 40-60% detection of all targets at 1 CFU/25 g meeting AOAC requirements.

Raw Ground Beef Validation (1 CFU/25 g)

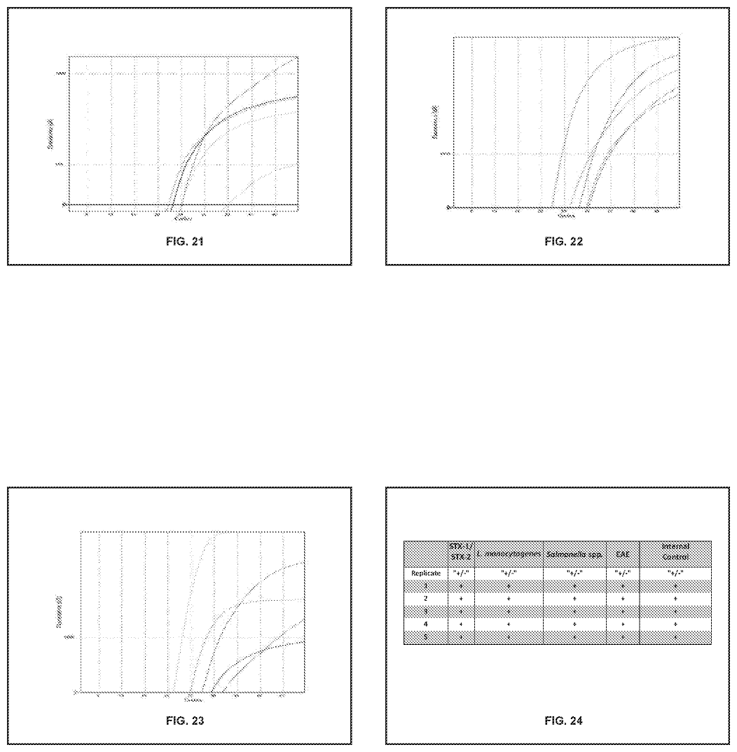

A low-level inoculation of 5 replicates of 5 CFU/25 g ground beef was performed (5 CFU inoculation is AOAC-required higher level of detection). All 3 target organisms were inoculated, stressed and enriched simultaneously with 7.00 CFU/25 g E. coli O157:H7 (ATCC:43895), 5.45 CFU/25 g S. enterica (ATCC:13076), 4.28 CFU/25 g L. monocytogenes (ATCC: 13932). All bacteria were inoculated into ground beef and stored at 4° C. for 48 hours as per AOAC guidelines. After 48 hours of incubation, samples were enriched at 37° C. for 24 hours in the selective enrichment media. An aerobic plate count (APC) was performed on an uninoculated control which indicated 5.1×10⁴ native bacteria per gram were present. A lysis procedure was performed to maximize recovery and detection of bacterial DNA, and amplification of the lysate was performed using an Agilent AriaMx Instrument to maximize recovery and detection of bacterial DNA. Primers and probes used are disclosed in Table 1. FIGS. 20-24 show the amplification curves for each target. FIG. 20—Raw Ground Beef 5 CFU/25 Gram Inoculation STX-1 and STX-2 Targets. The method detected STX-1 and STX-2 in 5/5 replicates (100% recovery). FIG. 21—Raw Ground Beef 5 CFU/25 Gram Inoculation E. coli EAE Target. The method detected E. coli EAE in 5/5 replicates (100% recovery). FIG. 22—Raw Ground Beef 5 CFU/25 Gram Inoculation L. monocytogenes Target. The method detected L. monocytogenes in 5/5 replicates (100% recovery). FIG. 23—Raw Ground Beef 5 CFU/25 Gram Inoculation S. enterica Target. The method detected S. enterica in 5/5 replicates (100% recovery). FIG. 24—Raw Ground Beef 5 CFU/25 Gram Inoculation All Targets Present. The method easily detects all targets when present in the same reaction. FIG. 25—Raw Ground Beef 5—CFU/25 g—Table of Results. The method detected all targets in 100% of replicates at a 5 CFU inoculation level.

Novel Listeria Spp. Target Set P.O.C. Cheese Study

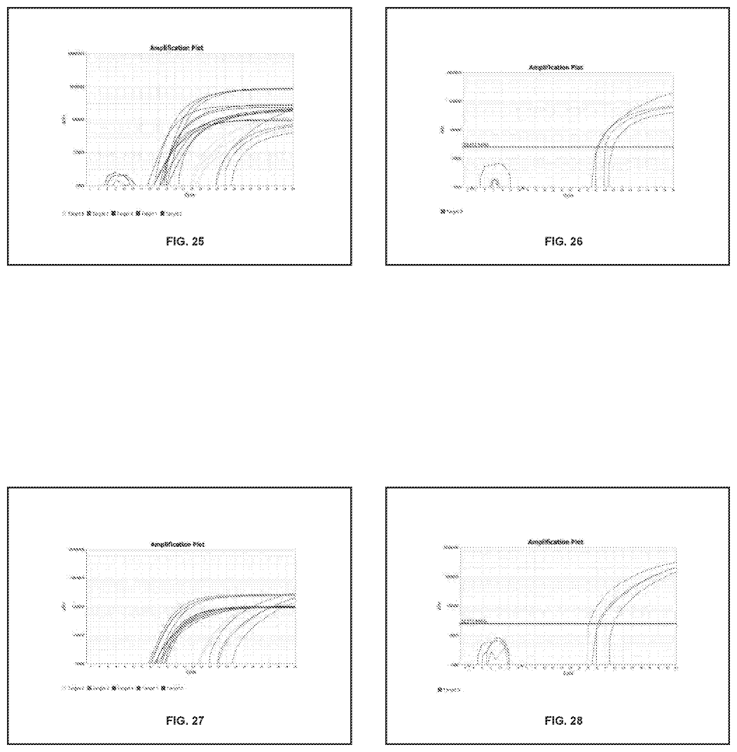

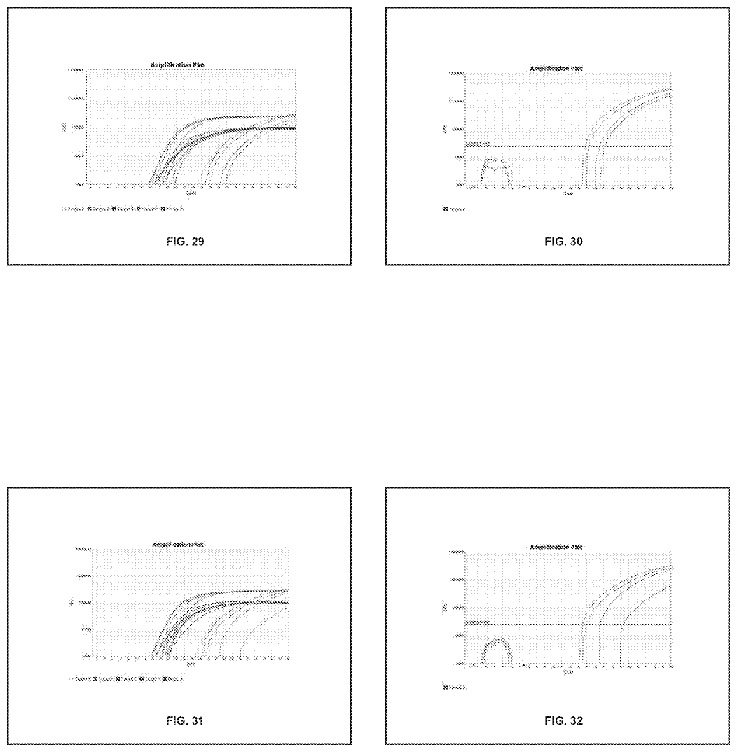

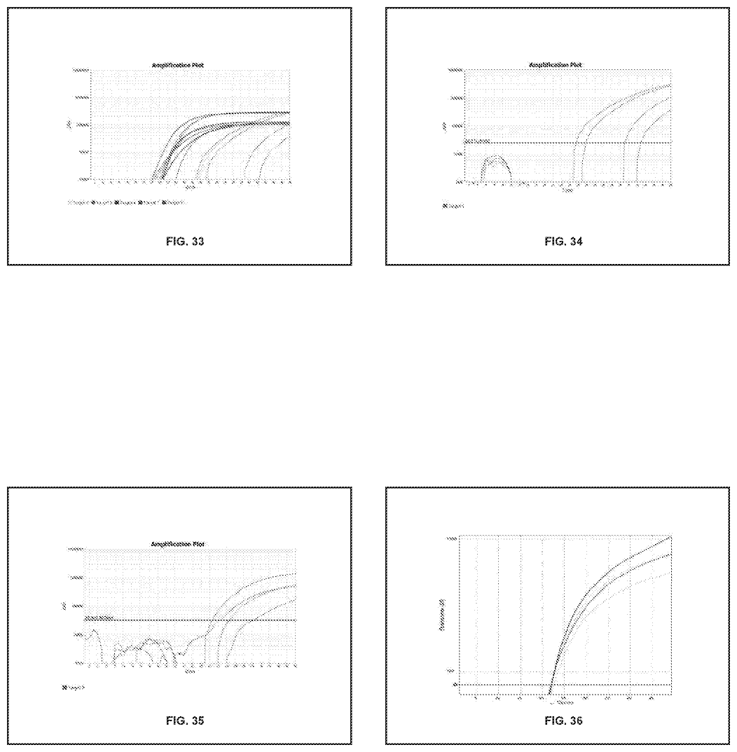

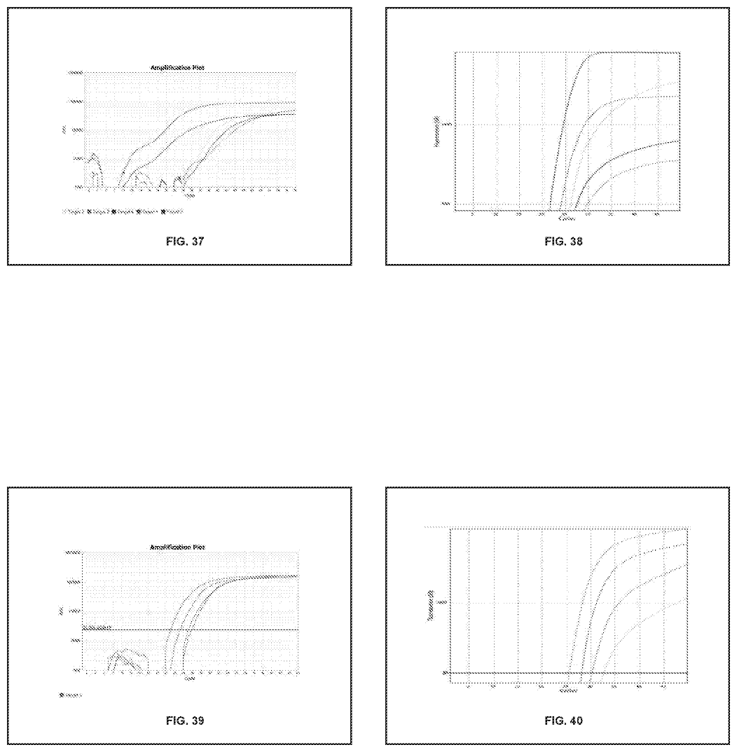

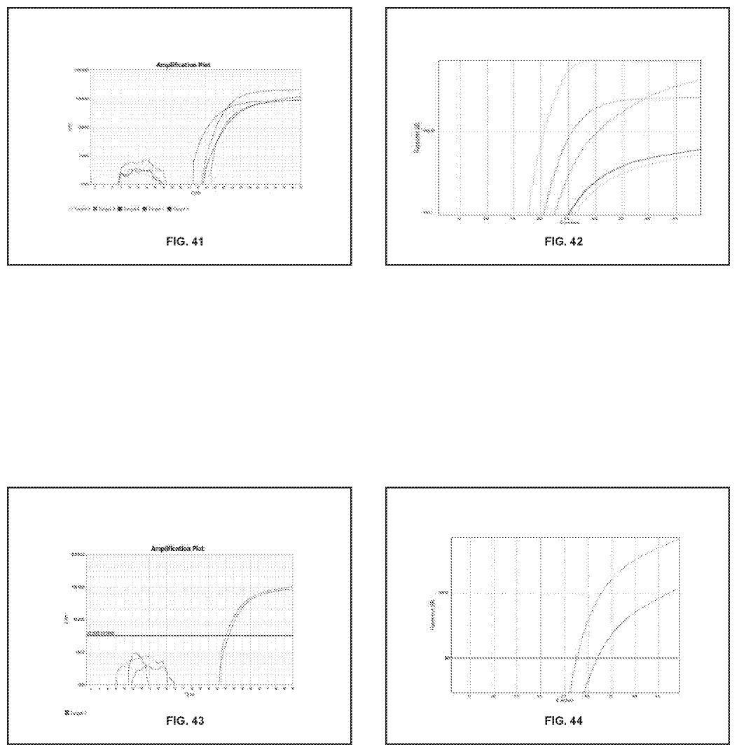

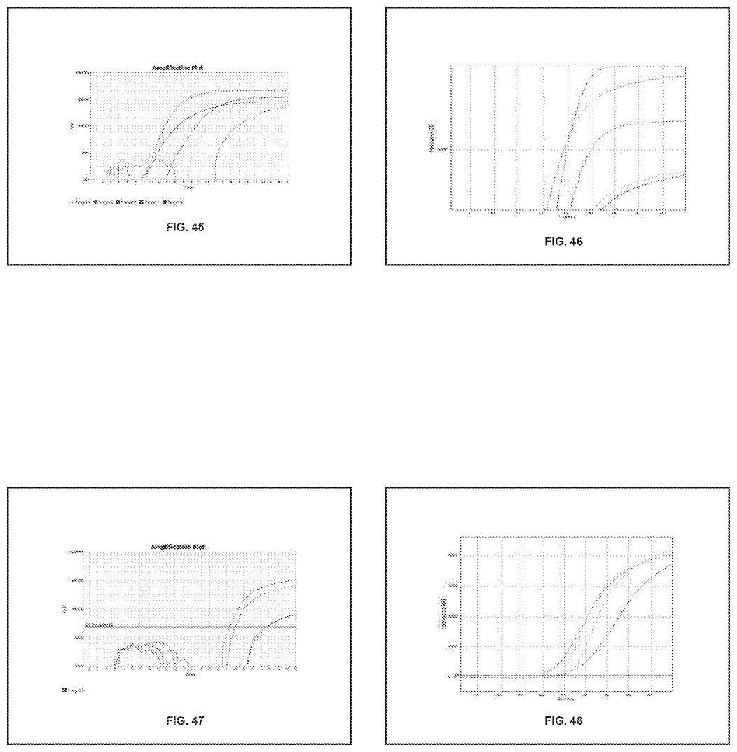

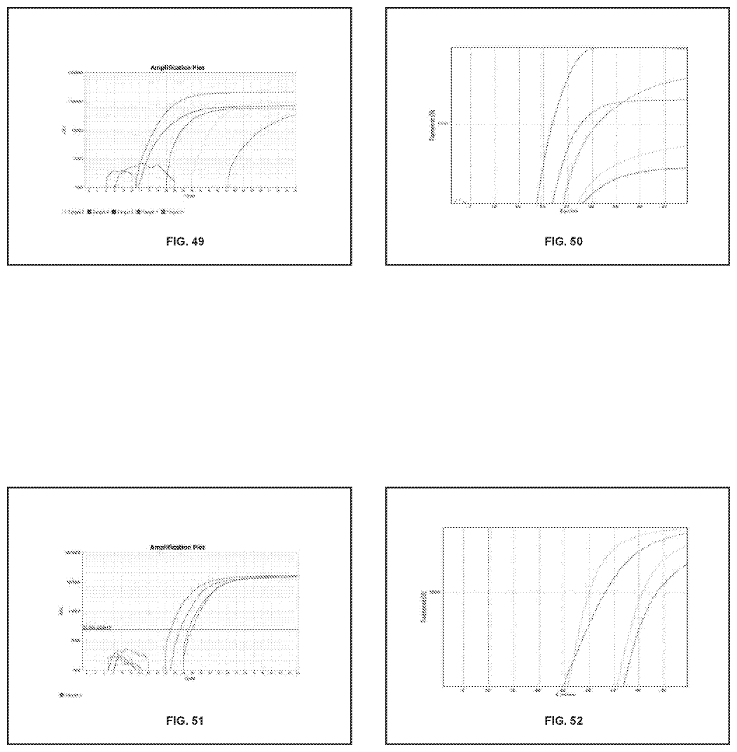

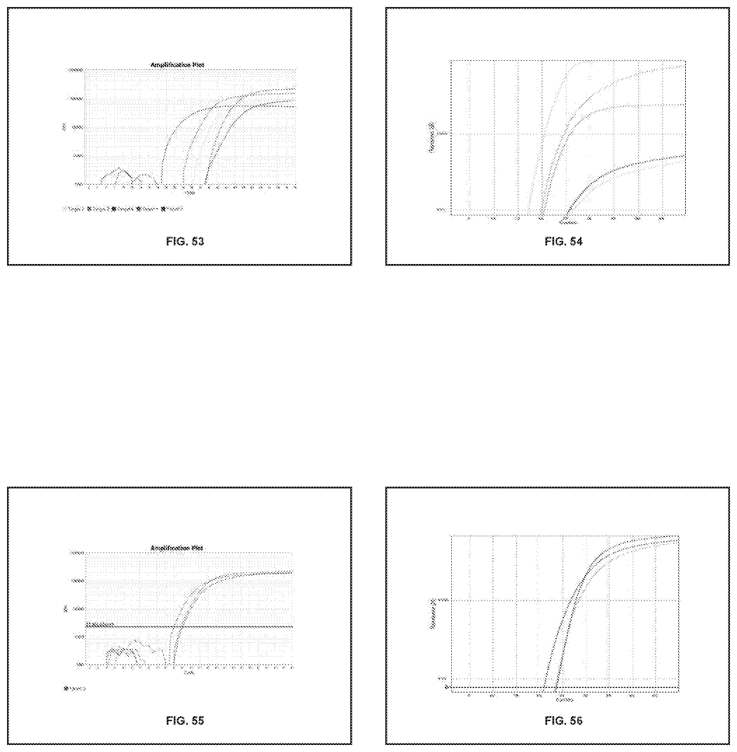

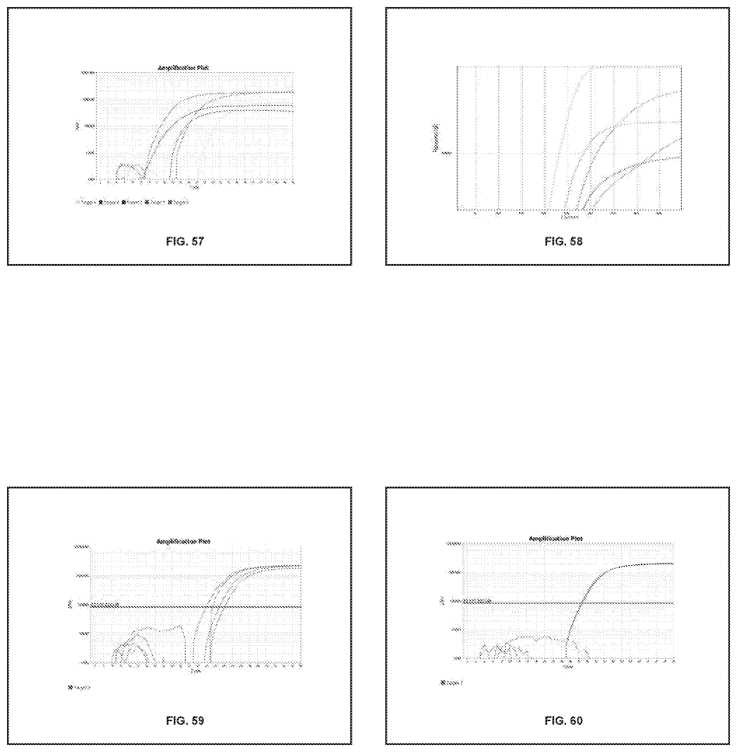

Novel Listeria spp. targets was liquid handling robotted into a multiplex (switchable with L. mono.) showing higher specificity, expanded scope necessary for expanded Listeria species, and 17 species total. These were the Sensu stricto: The original six species (L. mono, L. innocua, L. grayi, L. seeligeri, L. welshimeri, L. ivonovii), and Sensu lato: 11 new species described in recent years. All bacteria were inoculated into matrix and incubated at 37° C. All Listeria spp. target organisms were inoculated and enriched simultaneously; L. ivanovii (ATCC:19119), L. seeligeri (ATCC:35967), L. welshimeri (ATCC:35897), L. monocytogenes (ATCC:13932), and co-enrichment with all pathogen targets of the method; E. coli O157:H7 (ATCC:43895) and Salmonella enterica (ATCC:13076). A low-level inoculation was performed with 4 replicates of 2 CFU/25 g per matrix (2 CFU inoculation is within 0.2-2.0 CFU range of lower limit of detection) into 25 g matrix. For the high-level inoculation 4 replicates of 15 CFU/25 g per matrix were inoculated with 15 CFU to demonstrate efficacy in high-titer levels of pathogens into 25 g matrix. Samples were enriched at 37° C. for 24 hours and the lysis procedure performed to maximize recovery and detection of bacterial DNA. An assay was run on the lysate and real time PCR detection of nucleic acid targets was performed on a ThermoFisher ABI QuantStudio 5 (96-well format). Primers and probes used are disclosed in Table 1. FIG. 26—All four Listeria strains, 15 CFU with all targets selected. FIG. 27—All four Listeria strains, 15 CFU with Listeria spp. target only selected. FIG. 28—Listeria ivanovii, 2 CFU with all targets selected. FIG. 29—Listeria ivanovii only, 2 CFU. All targets selected. FIG. 30—Listeria monocytogenes, 2 CFU. Listeria spp. target only selected. FIG. 31—Listeria seeligeri, 2 CFU with all targets selected. FIG. 32—Listeria seeligeri, 2 CFU with Listeria spp. target only selected. FIG. 33—Listeria welshimeri, 2 CFU with all targets selected. FIG. 34—Listeria welshimeri, 2 CFU with Listeria spp. target only selected. The internal control was detected on all replicates, and 100% detection of all targets was achieved at 2 CFU/25 g & 15 CFU. FIG. 35—Cheddar—L. ivanovii 2 CFU. The method detected L. ivanovii in 4/4 replicates (100%). FIG. 36—Cheddar—L. ivanovii 2 CFU. The method detected L. ivanovii in 4/4 replicates (100%). FIG. 37—Cheddar—L. ivanovii 2 CFU. All targets selected. FIG. 38—Cheddar—L. ivanovii 2 CFU. All targets selected. FIG. 39—Cheddar—L. welshimeri 2 CFU. The method detected L. welshimeri in 4/4 replicates (100%). FIG. 40—Cheddar—L. welshimeri 2 CFU. The method detected L. welshimeri in 4/4 replicates (100%). FIG. 41—Cheddar—L. welshimeri 2 CFU. All targets selected. FIG. 42—Cheddar—L. welshimeri 2 CFU with all targets selected. FIG. 43—Cheddar L. monocytogenes 2 CFU. The method detected L. monocytogenes in 2/4 replicates (50%). FIG. 44—Cheddar—L. monocytogenes 2 CFU. The method detected L. monocytogenes in 2/4 replicates (50%). FIG. 45—Cheddar—L. monocytogenes 2 CFU with all targets selected. FIG. 46—Cheddar L. monocytogenes 2 CFU with all targets selected. FIG. 47—Ricotta L. ivanovii 2 CFU. The method detected L. ivanovii in 4/4 replicates (100%). FIG. 48—Ricotta L. ivanovii 2 CFU. The method detected L. ivanovii in 4/4 replicates (100%). FIG. 49—Ricotta L. ivanovii 2 CFU with all targets selected. FIG. 50—Ricotta—L. ivanovii 2 CFU with all targets selected. FIG. 51—Ricotta—L. welshimeri 2 CFU. The method detected L. welshimeri in 4/4 replicates (100%). FIG. 52—Ricotta—L. welshimeri 2 CFU. The method detected L. welshimeri in 4/4 replicates (100%). FIG. 53—Ricotta—L. welshimeri 2 CFU with all targets selected. FIG. 54—Ricotta—L. welshimeri, 2 CFU with all targets selected. FIG. 55—Ricotta—L. monocytogenes, 2 CFU. The method detected L. monocytogenes in 3/4 replicates (75%). FIG. 56—Ricotta—L. monocytogenes, 2 CFU. The method detected L. monocytogenes in 3/4 replicates (75%). FIG. 57—Ricotta—L. monocytogenes, 2 CFU with all targets selected. FIG. 58—Ricotta—L. monocytogenes, 2 CFU with all targets selected. FIG. 59—L. innocua Data—Deli Turkey. The method detected L. innocua in 4/4 replicates (100%). FIG. 60—L. innocua Data—Ricotta Cheese. The method detected L. innocua in 3/4 replicates (75%). FIG. 61—L. welshimeri Data—Deli Turkey. The method detected L. welshimeri in 3/4 replicates (75%). FIG. 62—L. welshimeri Data—Ricotta Cheese. The method detected L. welshimeri in 4/4 replicates (100%). FIG. 63—P.O.C. Cheese Study—The method Results.

QuantStudio5 and AriaMX Matrix Validation Set

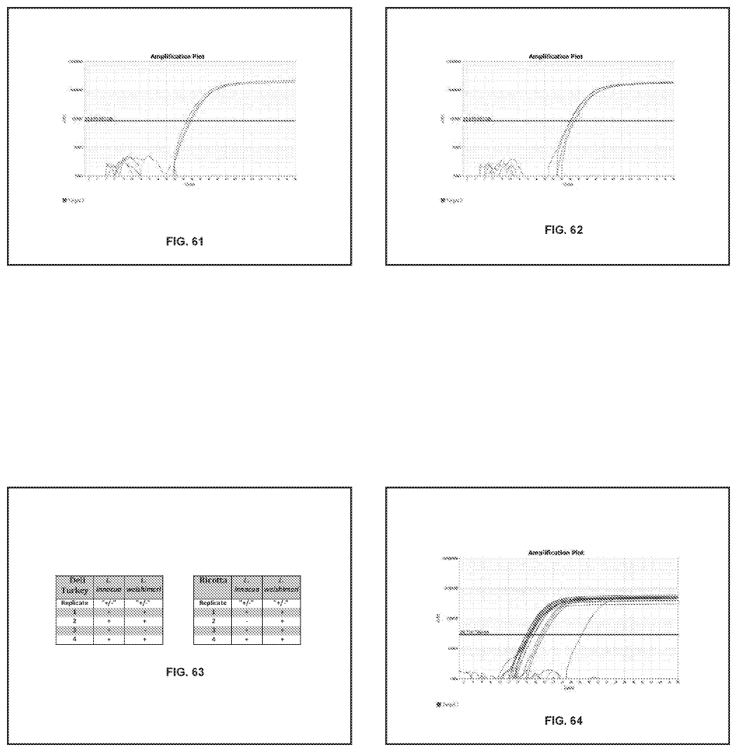

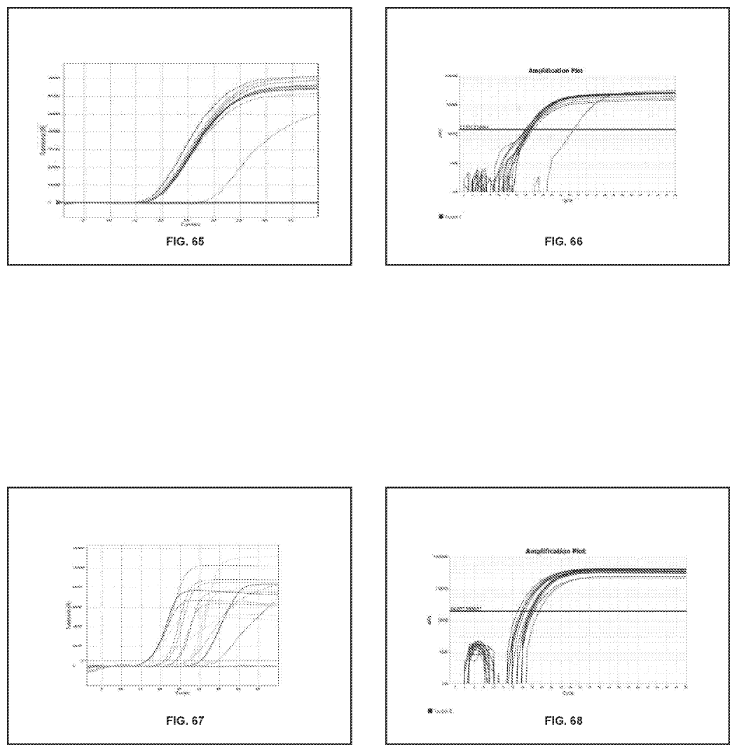

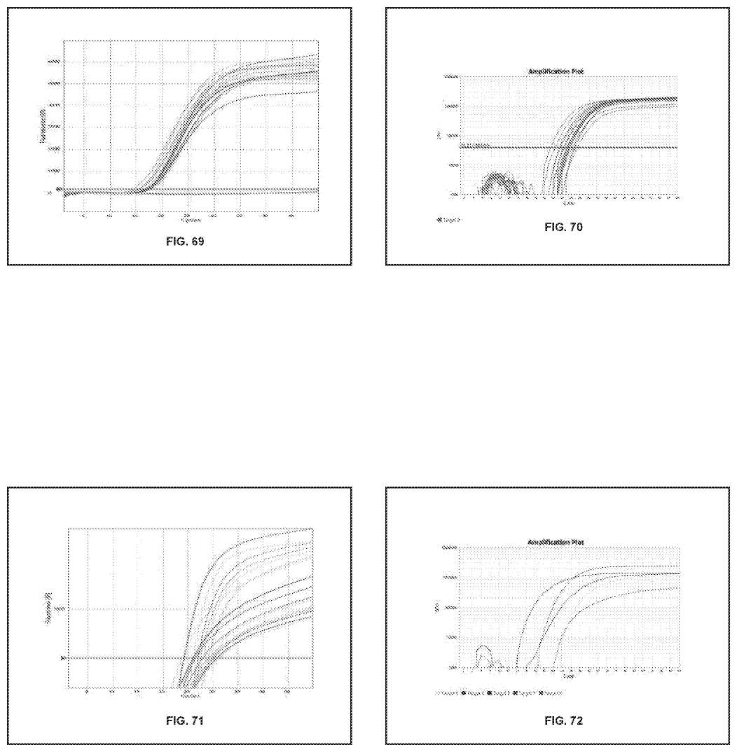

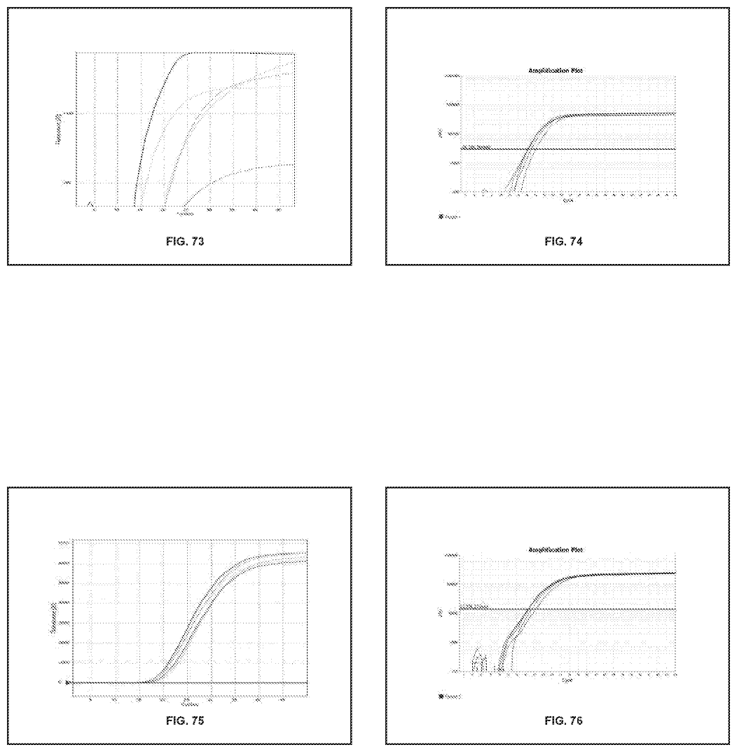

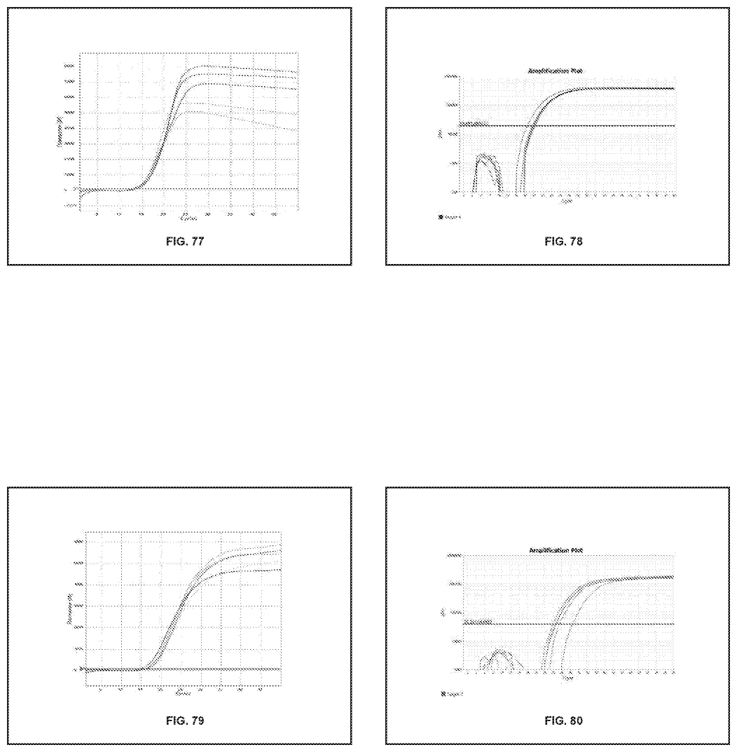

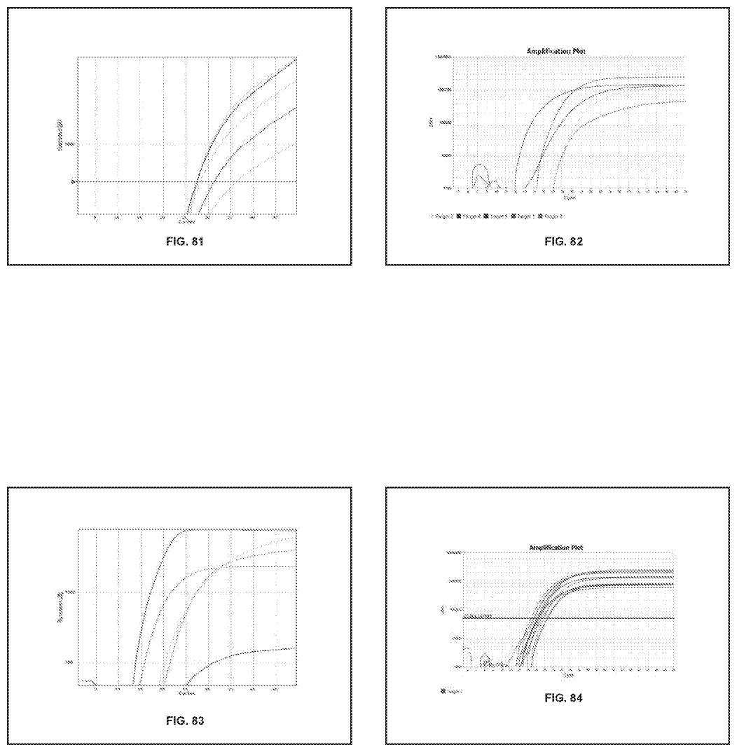

The AOAC mandates a two-tiered fractional recovery procedure. 1) A 1 CFU/25 g low level recovery target between 25%-75%, where results <25% or >75% are considered invalid. A 5 CFU/25 g high level recovery target where 100% recovery is required for 5 CFU inoculations. All bacteria were inoculated into the matrix and incubated at 37° C. All target organisms were inoculated and enriched simultaneously; L. welshimeri (ATCC:35897), E. coli O157:H7 (ATCC:43895), and S. enterica (ATCC:13076). For the low-level inoculation: 20 replicates of 1 CFU/25 g were inoculated per matrix where 1 CFU inoculation is within 0.2-2.0 CFU range of lower limit of detection, inoculated into 25 g matrix. All 3 target organisms were inoculated and incubated simultaneously with 1.28 CFU/25 g E. coli O157:H7 (ATCC:43895), 2.08 CFU/25 g S. enterica (ATCC:13076), and 2.33 CFU/25 g L. welshimeri (ATCC: 13932). For the high-level Inoculation 5 replicates of 5 CFU/25 g per matrix were used with 5 CFU inoculation to demonstrate efficacy in high-titer levels of pathogens, inoculated into 25 g matrix. All 3 target organisms inoculated and incubated simultaneously with 6.4 CFU/25 g E. coli O157:H7 (ATCC:43895), 10.4 CFU/25 g S. enterica (ATCC:13076), and 11.66 CFU/25 g L. welshimeri (ATCC: 13932). The samples were enriched at 37° C. for 24 hours in the selective enrichment media and an aerobic plate count (APC) performed on an uninoculated control to determine native bacteria per gram. A lysis procedure was performed to maximize recovery and detection of bacterial DNA and the assay run on lysate with real time PCR detection of nucleic acid targets on each of an ABI QuantStudio5 and an Agilent AriaMX. Primers and probes used are disclosed in Table 1. FIG. 64—QuantStudio5, Deli Turkey—1 CFU E. coli O157:H7 STEC STX-1 and STX-2. The method detected STX-1 and STX-2 targets in 15/20 replicates (75% recovery). FIG. 65—AriaMX, Deli Turkey—1 CFU E. coli O157:H7 STEC STX-1 and STX-2. The method detected STX-1 and STX-2 targets in 15/20 replicates (75% recovery). FIG. 66—QuantStudio5, Deli Turkey—1 CFU E. coli O157:H7 STEC EAE target. The method detected E. coli EAE in 15/20 replicates (75% recovery). FIG. 67—AriaMX, Deli Turkey—1 CFU E. coli O157:H7 STEC EAE target. The method detected E. coli EAE in 15/20 replicates (75% recovery). FIG. 68—QuantStudio5, Deli Turkey 1 CFU S. enterica target. The method detected the S. enterica target in 18/20 replicates (90% recovery). FIG. 69—AriaMX, Deli Turkey—1 CFU S. enterica target. The method detected the S. enterica target in 18/20 replicates (90% recovery). FIG. 70—QuantStudio5, Deli Turkey 1 CFU Listeria spp. target. The method detected the L. welshimeri target in 19/20 replicates (95% recovery). FIG. 71—AriaMX, Deli Turkey—1 CFU Listeria spp. Target. The method detected the L. welshimeri target in 19/20 replicates (95% recovery). FIG. 72—QuantStudio5, Deli Turkey 1 CFU All Targets. FIG. 73—AriaMX, All Targets—1 CFU. FIG. 74—QuantStudio5, Deli Turkey 5 CFU E. coli O157:H7 STEC STX-1 and STX-2. The method detected STX-1 and STX-2 in 5/5 replicates (100% recovery). FIG. 75—AriaMX, Deli Turkey. 5 CFU E. coli O157:H7 STEC STX-1 and STX-2. The method detected STX-1 and STX-2 in 5/5 replicates (100% recovery). FIG. 76—QuantStudio5, Deli Turkey 5 CFU E. coli O157:H7 STEC EAE target. The method detected E. coli EAE in 5/5 replicates (100% recovery). FIG. 77—AriaMX, Deli Turkey 5 CFU E. coli O157:H7 STEC EAE target. The method detected E. coli EAE in 5/5 replicates (100% recovery). FIG. 78—QuantStudio5, Deli Turkey 5 CFU S. enterica target. The method detected S. enterica in 5/5 replicates (100% recovery). FIG. 79—AriaMX, Deli Turkey—5 CFU S. enterica target. The method detected S. enterica in 5/5 replicates (100% recovery). FIG. 80—QuantStudio5, Deli Turkey 5 CFU Listeria spp. Target. The method detected L. welshimeri in 5/5 replicates (100% recovery). FIG. 81—AriaMX, Deli Turkey5 CFU Listeria spp. target. The method detected L. welshimeri in 5/5 replicates (100% recovery). FIG. 82—QuantStudio5, Deli Turkey 5 CFU All Targets Present. FIG. 83—AriaMX, Deli Turkey 5 CFU. All Targets Present.

Hemp (2 CFU/1 g & 15 CFU/1 g) Validation Data

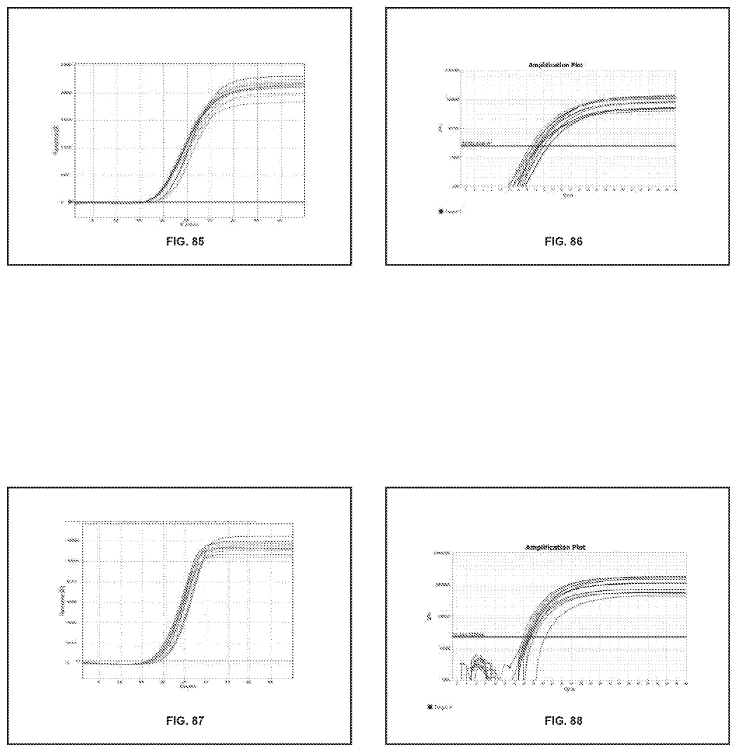

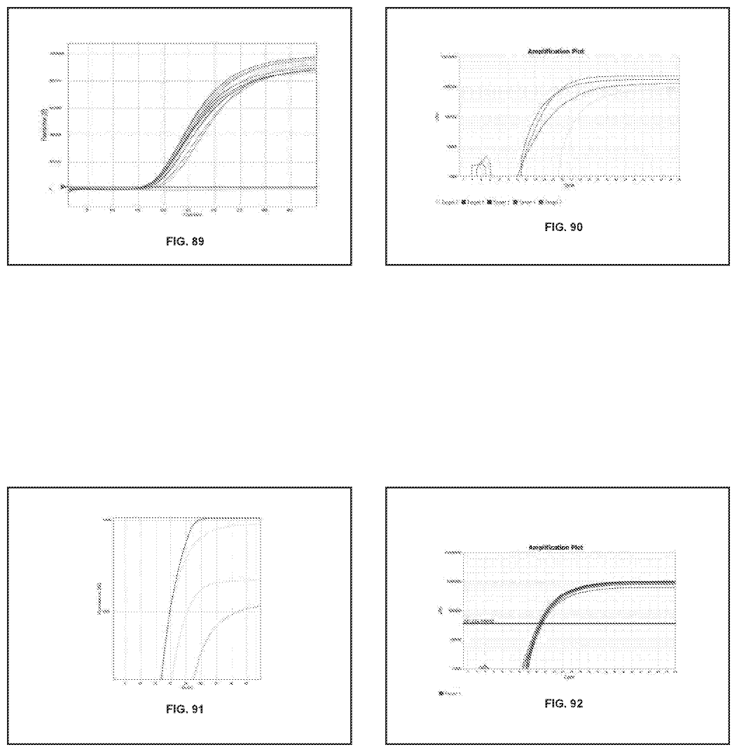

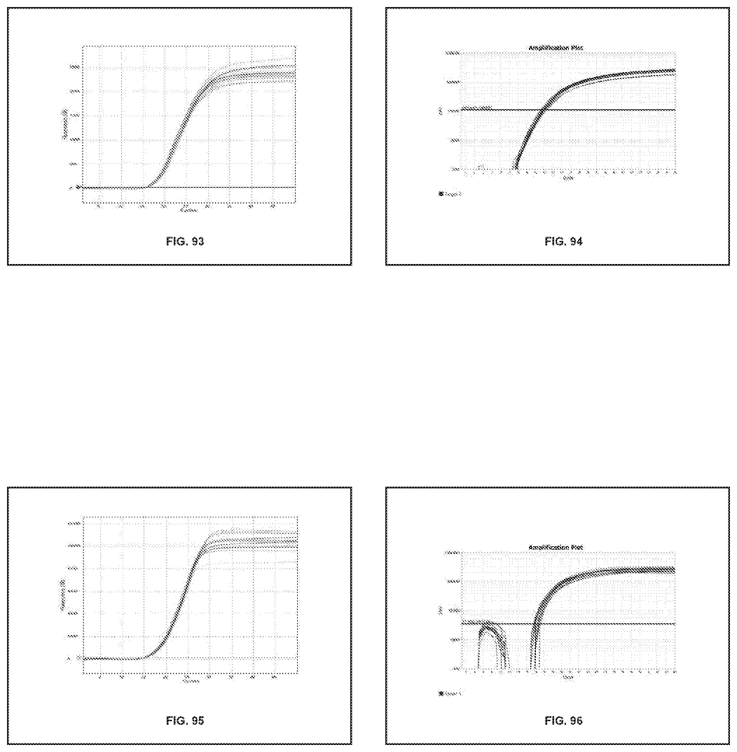

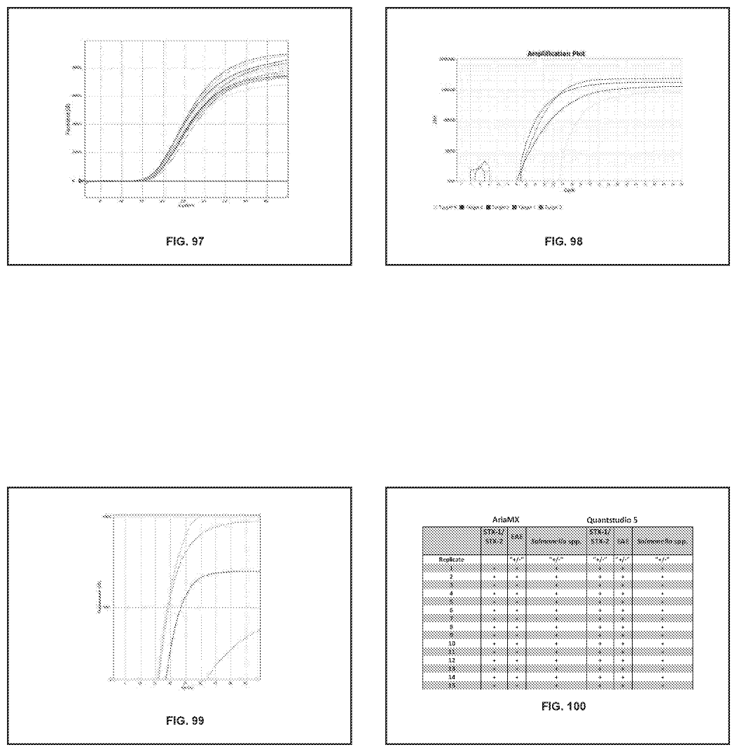

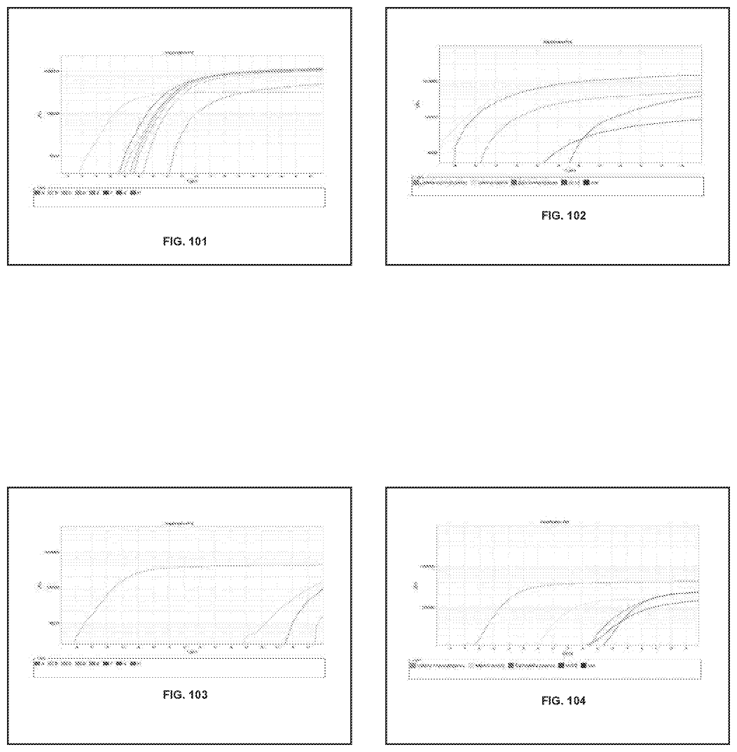

Bacteria were inoculated into a CBD-containing hemp strain (Suver Haze, Tweedle Farms) as a surrogate for Cannabis leaves and incubated at 37° C. All target organisms were inoculated and enriched simultaneously; E. coli O157:H7 (ATCC:43895), S. enterica (ATCC:13076), Low-level Inoculation: 15 replicates of 2 CFU/1 g per matrix, 2 CFU inoculation is within 0.2-2.0 CFU range of lower limit of detection, inoculated into 1 g of matrix. For high-level 15 replicates of 15 CFU/1 g were inoculated per matrix (to demonstrate efficacy in high-titer levels of pathogens) into 1 g matrix. Samples were enriched at 37° C. for 24 hours in enrichment media and a lysis procedure performed to maximize recovery and detection of bacterial DNA. The assay was run on the lysate with Real Time PCR detection of nucleic acid targets run on a ThermoFisher ABI QuantStudio 5 (96-well format) and an Agilent AriaMX (96-well format) in parallel. Primers and probes used are disclosed in Table 1. FIGS. 84-99 show the amplification curves. FIG. 84—QuantStudio5, Hemp—2 CFU E. coli O157:H7 STEC STX-1 and STX-2. The method detected STX-1 and STX-2 targets in 14/15 replicates (93% recovery). FIG. 85—AriaMX, Hemp—2 CFU E. coli O157:H7 STEC STX-1 and STX-2. The method detected STX-1 and STX-2 targets in 14/15 replicates (93% recovery). FIG. 86—QuantStudio5, Hemp—2 CFU E. coli O157:H7 STEC EAE target. The method detected E. coli EAE in 14/15 replicates (93% recovery). FIG. 87—AriaMX, Hemp—2 CFU E. coli O157:H7 STEC EAE target. FIG. 88—ABI QuantStudio5 Hemp—2 CFU S. enterica target. The method detected the S. enterica target in 14/15 replicates (93% recovery). FIG. 89—AriaMX, Hemp—2 CFU S. enterica target. The method detected the S. enterica target in 14/15 replicates (93% recovery). FIG. 90—QuantStudio5 All Targets in a Single Reaction—2 CFU. Example of the method detecting all pathogen targets in a single enrichment and PCR reaction. FIG. 91—AriaMX, Hemp All Targets in a Single Reaction—2 CFU. Example of detecting all pathogen targets in a single enrichment and PCR reaction. FIG. 92—QuantStudio5, Hemp—15 CFU E. coli O157:H7 STEC STX-1 and STX-2. The method detected STX-1 and STX-2 in 15/15 replicates (100% recovery). FIG. 93—AriaMX, Hemp—15 CFU E. coli O157:H7 STEC STX-1 and STX-2. The method detected STX-1 and STX-2 in 15/15 replicates (100% recovery). FIG. 94—QuantStudio5, Hemp—15 CFU E. coli O157:H7 STEC EAE target. The method detected E. coli EAE in 5/5 replicates (100% recovery). FIG. 95—AriaMX, Hemp—15 CFU E. coli O157:H7 STEC EAE target. The method detected E. coli EAE in 15/15 replicates (100% recovery). FIG. 96—QuantStudio5, Hemp—15 CFU S. enterica target. The method detected S. enterica in 15/15 replicates (100% recovery). FIG. 97—AriaMX, Hemp—15 CFU S. enterica target. The method detected S. enterica in 15/15 replicates (100% recovery). FIG. 98—QuantStudio5, Hemp 15 CFU All Targets Present. The method easily detects all targets when present in the same reaction. FIG. 99—AriaMX, Hemp 15 CFU All Targets Present. The method easily detects all targets when present in the same reaction. FIG. 100—Results Table AriaMx and Quantstudio5, 15 CFU/g. 100% agreement on endpoint detection for all targets across both instruments.

Hemp (2 CFU/1 g & 15 CFU/1 g) Validation Data

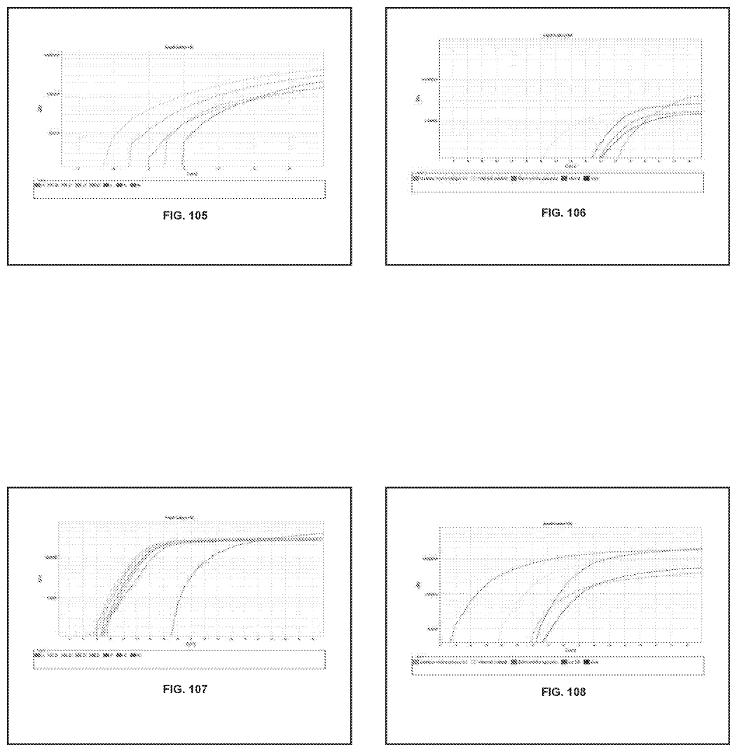

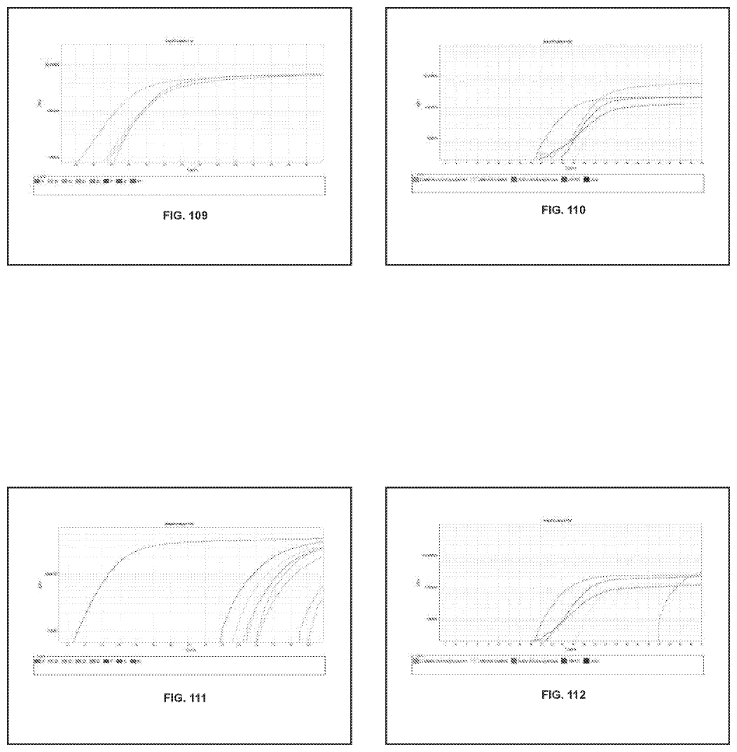

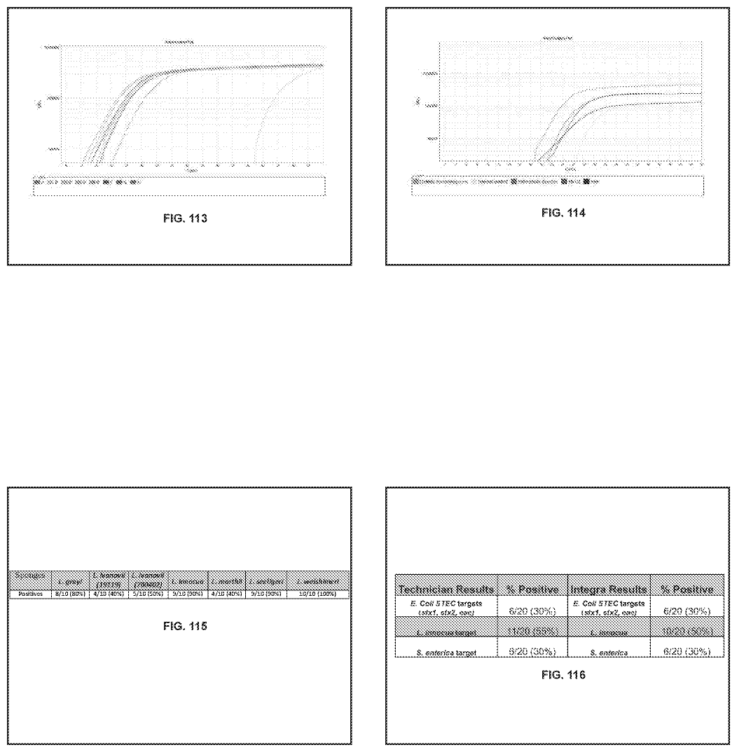

All bacteria were inoculated in a matrix and incubated at 37° C. All Listeria spp. target organisms were inoculated and enriched simultaneously which included: L. grayi (ATCC:19120), L. ivanovii (ATCC:19119), L. ivanovii (ATCC:700402), L. innocua, (ATCC:33090), L. marthii(BPBAA 1595), L. seeligeri (ATCC:35967), L. welshimeri (ATCC:35897). Co-enrichment was performed with all pathogen targets of the method which included: E. coli O157:H7 (ATCC:43895) and Salmonella enterica (ATCC:13076). A low-level inoculation was performed of 10 replicates of 1 CFU per sponge (1 CFU inoculation is within 0.2-2.0 CFU range of lower limit of detection). Samples were enriched at 37° C. for 24 hours in the selective enrichment media. Samples were lysed using the lysis procedure to maximize recovery and detection of bacterial DNA. The method was performed on the lysate using real time PCR detection of nucleic acid targets on a ThermoFisher ABI 7500 Fast (96-well format) machine. Primers and probes used are disclosed in Table 1. FIGS. 100-115 show the amplification curves. FIG. 101—Sponge—1 CFU L. grayi ATCC 19120, 8/10 replicates were positive for L. grayi at 1 CFU. FIG. 102—Sponge—1 CFU L. grayi ATCC19120. All targets present in one reaction FIG. 103—Sponge—1 CFU L. ivanovii ATCC 19119. 4/10 replicates positive for L. ivanovii at 1 CFU. FIG. 104—Sponge—1 CFU L. ivanovii ATCC 19119. All targets present in one reaction. FIG. 105—Sponge—1 CFU L. ivanovii ATCC 700402. 5/10 replicates positive for L. ivanovii at 1 CFU. FIG. 106—Sponge—1 CFU L. ivanovii ATCC 700402. All targets present in a single reaction. FIG. 107—Sponge—1 CFU L. innocua ATCC 33090. 9/10 replicates positive for L. innocua at 1 CFU. FIG. 108—Sponge—1 CFU L. innocua ATCC 33090. All targets present in a single reaction. FIG. 109—Sponge—1 CFU L. marthii BPBAA 1595. 4/10 replicates positive for L. marthii at 1 CFU. FIG. 110—Sponge—1 CFU L. marthii BPBAA 1595. All targets present in a single reaction. FIG. 111—Sponge—1 CFU L. seeligeri ATCC 35967. 9/10 replicates positive for L. seeligeri at 1 CFU. FIG. 112—Sponge—1 CFU L. seeligeri ATCC 35967. All targets present in a single reaction. FIG. 113—Sponge—1 CFU L. welshimeri ATCC 35897. 10/10 replicates positive for L. welshimeri at 1 CFU. FIG. 114—Sponge—1 CFU L. welshimeri ATCC 35897. All targets present in a single reaction. FIG. 115—Sponge Study—Listeria spp. on ABI 7500. Table of results for Listeria spp. on ABI 7500.

Pork Sausage (1 CFU/25 g) Validation Data

All target organisms were inoculated and enriched simultaneously; E. coli O157:H7 (ATCC:43895), S. enterica (ATCC:13076) and L. innocua (ATCC:33090). All bacteria were inoculated into pork sausage and stored at 4° C. for 48 hours, as per AOAC guidelines. Aerobic plate counts (APC) had less than 10 CFU/gram. Samples were enriched at 37° C. for 24 hours in 22 ml of PMEM. A lysis procedure as previously described herein was performed to maximize recovery and detection of target bacteria DNA. Amplification and detection was performed on a multiplex qPCR assay run on an Applied Biosystems™ 7500 Fast machine. Up to 288 tests were run on each 96-well PCR plate. 1,150 tests were run using 384 well format.

A low-level inoculation was performed of 20 replicates of 1 CFU/25 g of pork sausage: 1.37 CFU/25 g E. coli O157:H7 (ATCC:43895), 1.13 CFU/25 g S. enterica (ATCC:13076), 1.5 CFU/25 g L. innocua (ATCC:33090). 1 CFU inoculation is AOAC-required lower limit of detection.

A high-level inoculation was performed of 5 replicates of 5 CFU/25 g of pork sausage: 6.85 CFU/25 g E. coli O157:H7 (ATCC:43895), 5.65 CFU/25 g S. enterica (ATCC:13076), 7.5 CFU/25 g L. innocua (ATCC:33090).

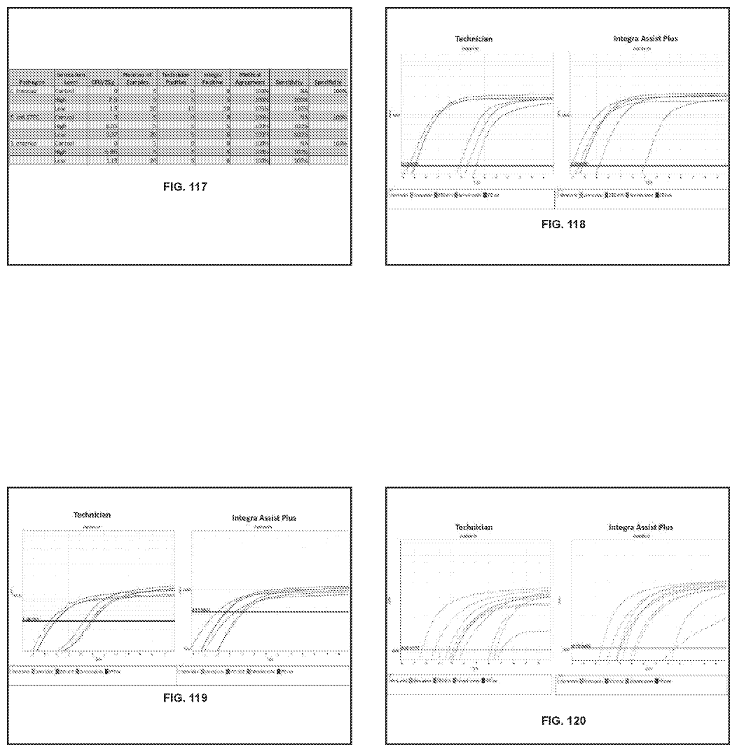

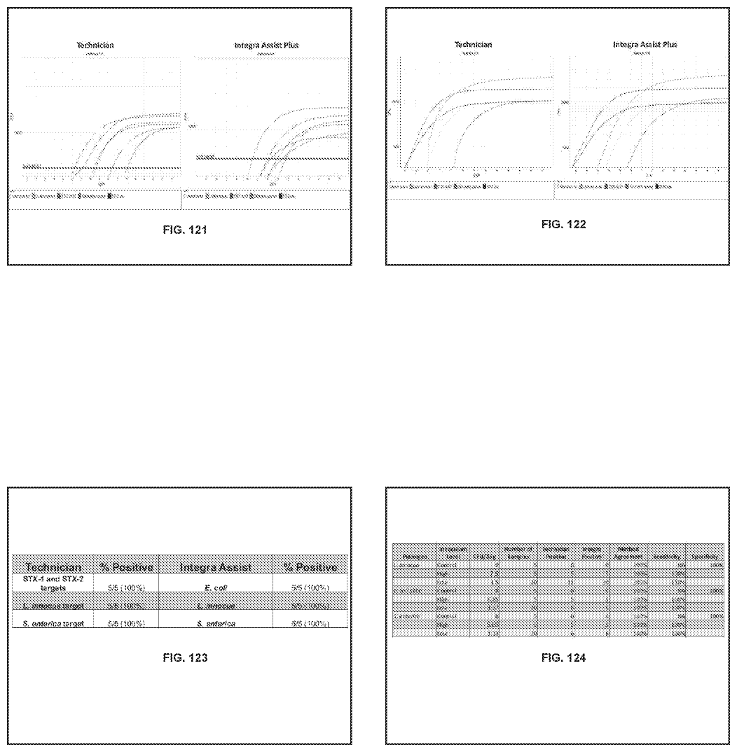

Samples were enriched at 37° C. for 24 hours in the selective enrichment media. Samples were lysed using the lysis procedure to maximize recovery and detection of bacterial DNA. The method was performed on the lysate using real time PCR detection of nucleic acid targets on a ThermoFisher™ ABI 7500 Fast (96-well format) machine. Primers and probes used are disclosed in Table 1. FIG. 116 shows a table summarizing the results for Liquid handling robot and Technician run for samples of 1 CFU/25 g Pork Sausage on QuantStudio 5 and ABI 7500 Fast. FIG. 117 shows a table of results for Liquid handling robot Validation. FIG. 118 shows Liquid handling robot Validation—1 CFU Pork Sausage E. coli O157:H7 ABI 7500 Fast. FIG. 119 shows Liquid handling robot Validation—1 CFU Pork Sausage E. coli O157:H7 ABI 7500 Fast. FIG. 120 shows Liquid handling robot Validation—1 CFU Pork Sausage L. innocua target ABI 7500 Fast. FIG. 121 shows Liquid handling robot Validation—1 CFU Pork Sausage S. enterica target ABI 7500 Fast. FIG. 122 shows Liquid handling robot Validation—1 CFU Pork Sausage All Targets Present Quantstudio ABI 7500 Fast.

Pork Sausage (5 CFU/25 g) Validation Data

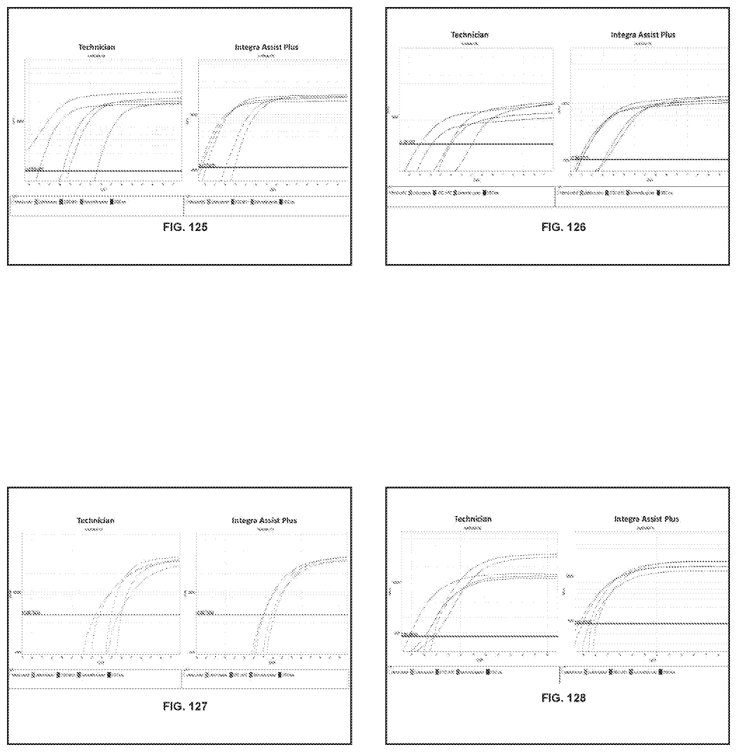

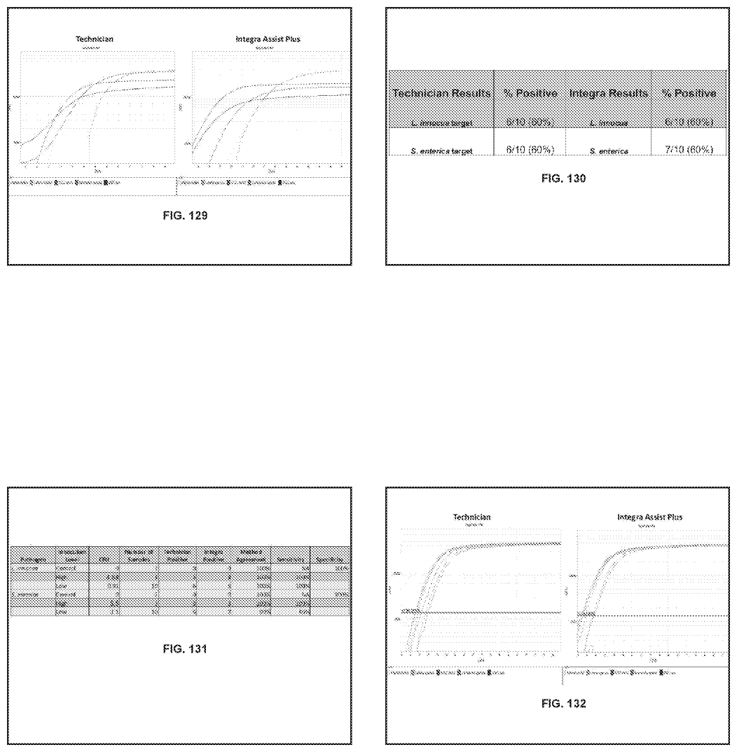

A high-level inoculation was performed of 5 replicates of 5 CFU/25 g of pork sausage (5 CFU is AOAC required higher level of inoculation): 3.42 CFU/25 g E. coli O157:H7 (ATCC:43895), 4.3 CFU/25 g S. enterica (ATCC:13076), 5.5 CFU/25 g L. innocua (ATCC:33090). All target organisms were inoculated and enriched simultaneously; E. coli O157:H7 (ATCC:43895), S. enterica (ATCC:13076) and L. innocua (ATCC:33090). All bacteria were inoculated into pork sausage and stored at 4° C. for 48 hours, as per AOAC guidelines. Aerobic plate counts (APC) had less than 10 CFU/gram. Samples were then enriched at 37° C. for 24 hours in 22 ml of enrichment media. A lysis procedure as previously described herein was performed to maximize recovery and detection of target bacteria DNA. Amplification and detection was performed on a multiplex qPCR assay run on an ABI 750 Fast™ machine. Up to 288 tests were run on each 96-well PCR plate. 1,150 tests were run using 384 well format. FIGS. 123 and 124 show amplification curves of the targets. FIG. 123 shows a table of Results for Liquid handling robot and Technician Run Samples. FIG. 124 shows a table of results for Liquid handling robot Validation. In conclusion, for Technician PCR Results with E. coli (STEC) targets (3 targets), for STX-1/STX-2: the method identified 5/5 (100%) replicates as positive for STX-1/STX-2, for EAE: the method identified 5/5 (100%) replicates as positive EAE, for S. enterica (1 target): the method identified 5/5 (100%) replicates as positive for S. enterica. For L. innocua (1 target): the method identified 5/5 (100%) replicates as positive for L. innocua. For Liquid handling robot PCR Results, E. coli (STEC) targets (3 targets), STX-1/STX-2: the method identified 5/5 (100%) replicates as positive for STX-1/STX-2, EAE: the method identified 5/5 (100%) replicates as positive EAE, for S. enterica (1 target): the method identified 5/5 (100%) replicates as positive for S. enterica, for L. innocua (1 target): the method identified 5/5 (100%) replicates as positive for L. innocua. Overall 100% detection of all targets at 5 CFU/25 g was obtained, meeting AOAC recovery requirements. FIGS. 125-12 show amplification curves of the targets. FIG. 125 shows results for Liquid handling robot Validation—5 CFU Pork Sausage E. coli O157:H7 on ABI 7500 Fast. FIG. 126 shows Liquid handling robot Validation—5 CFU Pork Sausage E. coli O157:H7 ABI 7500 Fast. FIG. 127 shows Liquid handling robot Validation—5 CFU Pork Sausage L. innocua target ABI 7500 Fast. FIG. 128 shows Liquid handling robot Validation—5 CFU Pork Sausage S. enterica target ABI 7500 Fast. FIG. 129 shows Liquid handling robot Validation—5 CFU Pork Sausage All Targets Present Quantstudio ABI 7500 Fast.

Environmental Sponge Validation Study

All target organisms were inoculated and enriched simultaneously; S. enterica (ATCC:13076) and L. innocua (ATCC:33090). All bacteria were inoculated directly onto environmental sponges. 90 ml PMEM was used for each enrichment bag. Aerobic plate counts (APC) had less than 10 CFU/gram. Samples were enriched at 37° C. for 24 hours in 22 ml of PMEM. A lysis procedure as previously described herein was performed to maximize recovery and detection of target bacteria DNA. Amplification and detection was performed on a multiplex qPCR assay run on an ABI 750 Fast™ machine. Up to 288 tests were run on each 96-well PCR plate. 1,150 tests were run using 384 well format.

A low-level inoculation was performed of 20 replicates of 1 CFU/25 g of pork sausage: 1.37 CFU/25 g E. coli O157:H7 (ATCC:43895), 1.13 CFU/25 g S. enterica (ATCC:13076), 1.5 CFU/25 g L. innocua (ATCC:33090). 1 CFU inoculation is AOAC-required lower limit of detection.

A high-level inoculation was performed of 5 replicates of 5 CFU/25 g of pork sausage: 6.85 CFU/25 g E. coli O157:H7 (ATCC:43895), 5.65 CFU/25 g S. enterica (ATCC:13076), 7.5 CFU/25 g L. innocua (ATCC:33090).

Samples were enriched at 37° C. for 24 hours in the selective enrichment media. Samples were lysed using the lysis procedure to maximize recovery and detection of bacterial DNA. The method was performed on the lysate using real time PCR detection of nucleic acid targets on a ThermoFisher ABI 7500 Fast (96-well format) machine. Primers and probes used are disclosed in Table 1.

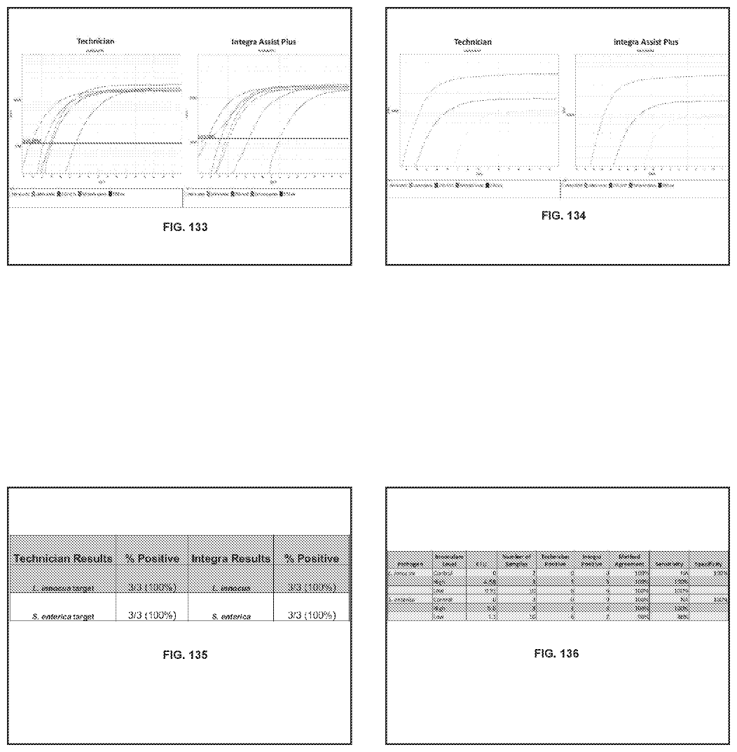

FIG. 130 shows Results for Liquid handling robot and Technician Run Samples 1 CFU/sponge on ABI 7500 Fast 5 CFU/25 g Pork Sausage on ABI 7500 Fast. FIG. 131 shows a table of results for Liquid handling robot Validation.

In conclusion, for the Liquid handling robot Validation at 1 CFU/sponge, the technician PCR results showed that for S. enterica with 1 target, the method identified 6/10 (60%) replicates as positive for S. enterica. For L. innocua (1 target): the method identified 6/10 (60%) replicates as positive for L. innocua. For the Liquid handling robot PCR Results S. enterica (1 target): the method identified 7/10 (70%) replicates as positive for S. enterica. L. innocua (1 target): the method identified 6/10 (60%) replicates as positive for L. innocua. Overall 60-70% detection of all targets at 1 CFU was achieved, meeting AOAC requirements. FIG. 132 shows Liquid handling robot Validation—1 CFU Sponge L. innocua target ABI 7500 Fast. FIG. 133 shows Liquid handling robot Validation—1 CFU Sponge S. enterica target. FIG. 134 shows Liquid handling robot Validation—1 CFU Sponge All Targets Present Quantstudio ABI 7500 Fast.

Liquid Handling Robot Validation Study—Environmental Sponges 5 CFU/Sponge

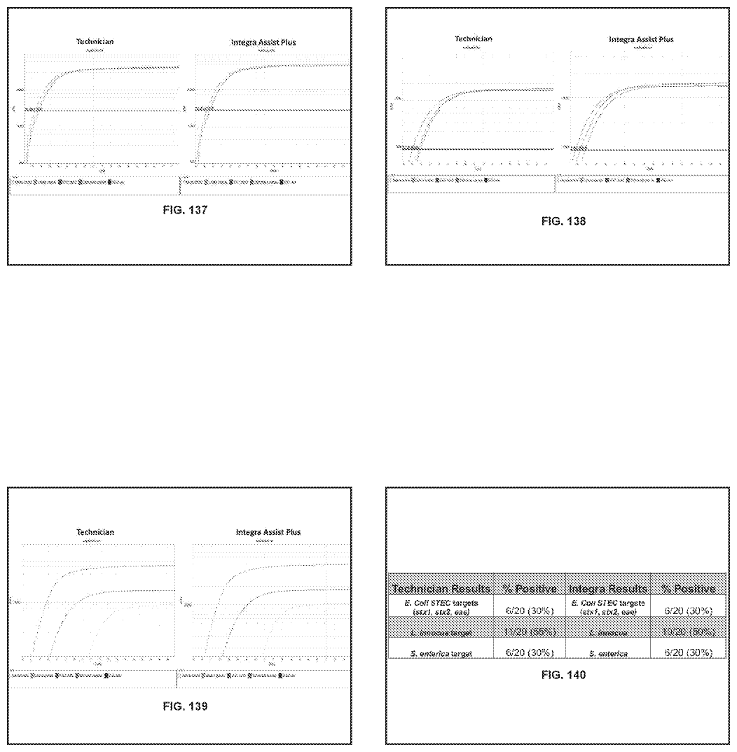

FIG. 135 shows Results for Liquid handling robot and Technician Run Samples 5 CFU/sponge on ABI 7500 Fast. FIG. 136 shows a table of results for Liquid handling robot Validation. The table shows 100% method agreement in detection of all targets at CFU with sensitivity and specificity meeting AOAC requirements. In conclusion for the technician PCR results S. enterica with one target, the method identified 3/3 (100%) replicates as positive for S. enterica. For L. innocua with one target the method identified 3/3 (100%) replicates as positive for L. innocua. For the Liquid handling robot PCR results S. enterica with one target the method identified 3/3 (100%) replicates as positive for S. enterica. For L. innocua with one target the method identified 3/3 (100%) replicates as positive for L. innocua. Overall the method showed 100% detection of all pathogens at 5 CFU. FIG. 137 shows Liquid handling robot Validation—5 CFU Sponge L. innocua target ABI 7500 Fast. The Liquid handling robot and technician-run samples both detected the Listeria spp. target in 3/3 replicates (100% recovery). FIG. 138 shows Liquid handling robot Validation—5 CFU Sponge S. enterica target ABI 7500 Fast. The Liquid handling robot and technician-run samples both detected the S. enterica target in 3/3 replicates (100% recovery). FIG. 139 shows Liquid handling robot Validation—5 CFU Sponge All Targets Present Quantstudio ABI 7500 Fast. All targets were present in a single reaction.

Liquid Handling Robot Validation Study—RTE Pork Sausage and Environmental Sponges

A liquid handling robot was used to prepare reactions. A novel Listeria spp. target set was used with Salmonella spp. and STEC E. coli multiplex in place of a L. monocytogenes-specific target. The Listeria spp. target set showed broad scope, detecting a large range of Listeria species including L. monocytogenes, L. innocua, L. grayi, L. welshimeri, L. marthii, L. ivanovii, and L. seeligeri. The target set was shown to work across multiple instrument platforms including QuantStudio 5, and ABI 7500 Fast. Liquid robot handling samples were directly compared to the same set of samples run by laboratory personnel. Both sets of samples were run on same 96-well PCR plate.

A low level inoculation was performed for 20 replicates at 1 CFU/25 g of cooked pork sausage. 1 CFU inoculation is the AOAC-required lower limit of detection. All three target organisms were inoculated and incubated simultaneously. 1.37 CFU/25 g of E. coli O157:H7 (ATC:43895), 1.13 CFU/25 g for S. enterica (ATCC:13076), and 1.5 CFU/25 g L. innocua (ATCC:33090). A high level inoculation was performed with 5 replicates of 5 CFU/25 g of pork sausage. All three target organisms were inoculated and incubated simultaneously. 6.85 CFU/25 g E. coli O157:H7 (ATCC:43895), 5.65 CFU/25 g S. enterica (ATCC:13076), 7.5 CFU/25 g L. innocua (ATCC:33090). All bacteria were inoculated into pork sausage and stored at 4° C. for 48 hours, as per AOAC guidelines. An aerobic plate count (APC) showed less than 10 CFU/gram. Samples were enriched at 37° C. for 24 hours in 225 ml of enrichment media. The lysis method was performed to maximize recovery and detection of target bacteria DNA. The multiplex qPCR assay was run on an ABI 7500 Fast. Up to 288 tests were performed per 96-well PCR plate. Up to 1,150 tests were performed using 384 well format.

FIG. 140 shows Results for Liquid handling robot and Technician Run Samples for 1 CFU/25 g Pork Sausage on Quantstudio 5 and ABI 7500 Fast. FIG. 141 shows a table of results for the liquid handling robot validation. 100% method agreement in detection of all targets at 1 CFU can be observed with sensitivity and specificity meeting validation requirements. In conclusion for the technician obtained PCR results, E. coli (STEC) targets (3 targets) STX-1/STX-2: the method identified 6/20 (30%) replicates as positive for STX-1/STX-2, for EAE: the method identified 6/20 (30%) replicates as positive EAE, for S. enterica (1 target): the method identified 6/20 (30%) replicates as positive for S. enterica, and for L. innocua with 1 target, the method identified 11/20 (55%) replicates as positive for L. innocua. In summary, 30-55% detection of all targets at 1 CFU/25 g was obtained, meeting AOAC fractional recovery requirements.

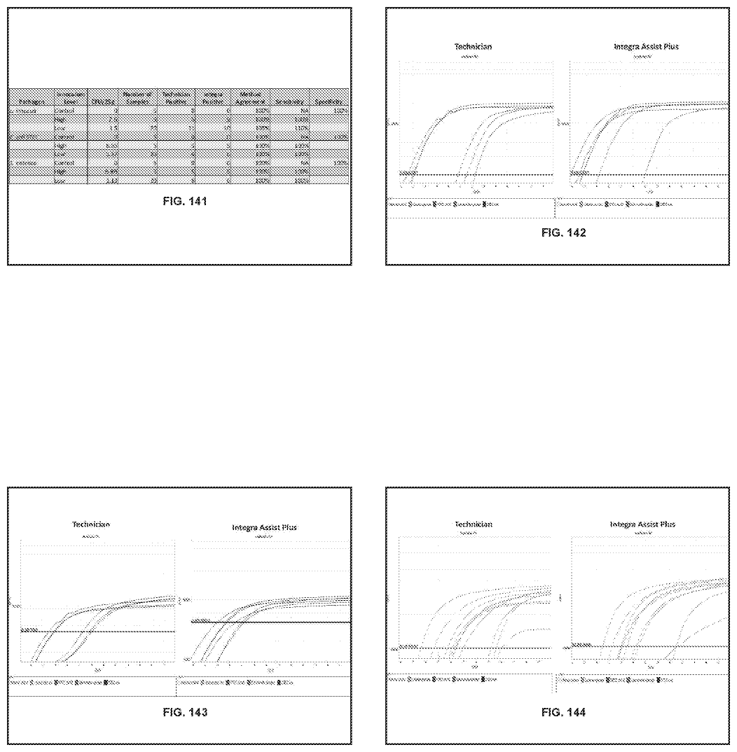

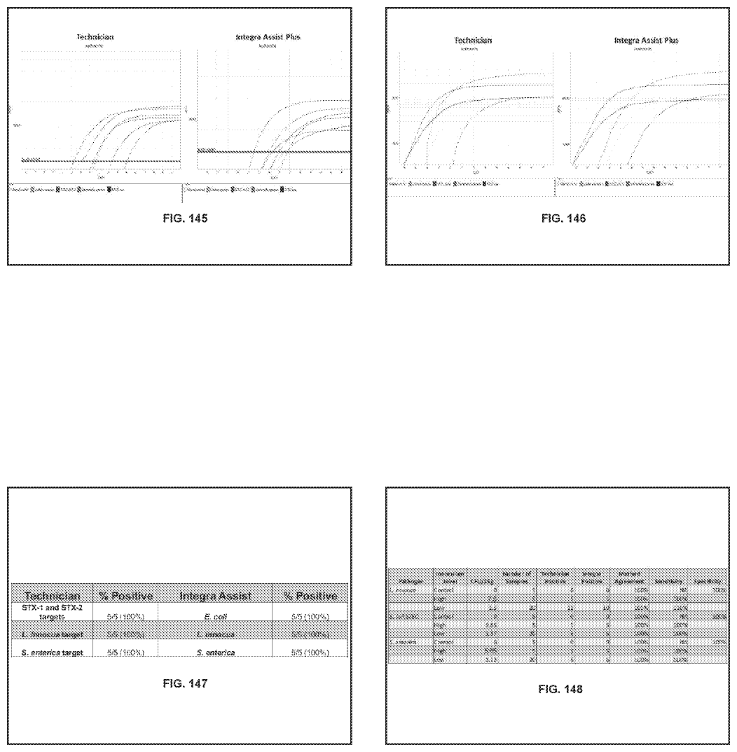

FIG. 142 shows Liquid handling robot Validation—1 CFU Pork Sausage E. coli O157:H7 ABI 7500 Fast. FIG. 143 shows Liquid handling robot Validation—1 CFU Pork Sausage E. coli O157:H7 ABI 7500 Fast. FIG. 144 shows Liquid handling robot Validation—1 CFU Pork Sausage L. innocua target ABI 7500 Fast. FIG. 145 shows Liquid handling robot Validation—1 CFU Pork Sausage S. enterica target ABI 7500 Fast. FIG. 146 shows Liquid handling robot Validation—1 CFU Pork Sausage All Targets Present Quantstudio ABI 7500 Fast.

Liquid Handling Robot Validation Study ABI 7500 Fast Pork Sausage 5 CFU/25 g

A high level inoculation was performed with 5 replicates of 5 CFU/25 g of pork sausage. 5 CFU in inoculation is the AOAC-required higher level of inoculation. All three target organisms were inoculated, stressed, and enriched simultaneously. 3.42 CFU/25 g E. coli O157:H7 (ATCC:43895), 4.30 CFU/25 g S. enterica (ATCC:13076), 5.50 CFU/25 g L. innocua (ATCC:33090). All bacteria were inoculated into pork sausage and stored at 4° C. for 48 hours, as per AOAC guidelines. Samples were enriched at 37° C. for 24 hours in 225 ml of enrichment media. An aerobic plate count (APC) showed less than 10 CFU/gram. The lysis method was performed to maximize recovery and detection of target bacteria DNA. The multiplex qPCR assay was run on the lysate using an ABI 7500 Fast. Up to 288 tests were performed per 96-well PCR plate. Up to 1,150 tests were performed using 384 well format.

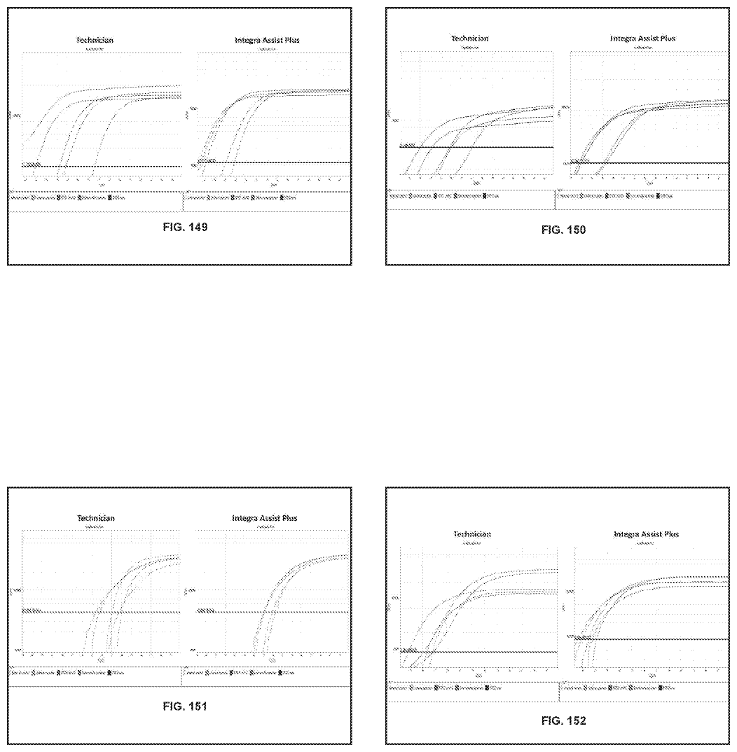

FIG. 147 shows results for Liquid handling robot and Technician Run Samples for 5 CFU/25 g Pork Sausage run on an ABI 7500 Fast. FIG. 148 shows Liquid handling robot Validation Table of Results. 100% method agreement in detection of all targets at 5 CFU was observed, with sensitivity and specificity meeting validation requirements. FIG. 149—Liquid handling robot Validation—5 CFU Pork Sausage E. coli O157:H7 ABI 7500 Fast. FIG. 150 shows Liquid handling robot Validation—5 CFU Pork Sausage E. coli O157:H7 ABI 7500 Fast. FIG. 151 shows Liquid handling robot Validation—5 CFU Pork Sausage L. innocua target ABI 7500 Fast. FIG. 152 shows Liquid handling robot Validation—5 CFU Pork Sausage S. enterica target ABI 7500 Fast. FIG. 153 shows Liquid handling robot Validation—5 CFU Pork Sausage All Targets Present Quantstudio ABI 7500 Fast.

Liquid Handling Robot Validation—Environmental Sponges

A low level inoculation was performed for 10 replicates at 1 CFU/sponge. 1 CFU inoculation is the AOAC-required lower limit of detection. Two target organisms were inoculated and incubated simultaneously. 1.10 CFU/sponge for S. enterica (ATCC:13076), and 0.91 CFU/sponge L. innocua (ATCC:33090). A high level inoculation was performed with 3 replicates of 5 CFU/sponge. Two target organisms were inoculated and incubated simultaneously. 5.5 CFU/sponge enterica (ATCC:13076), and 4.58 CFU/sponge L. innocua (ATCC:33090). All bacteria were inoculated directly onto the sponge and enriched in 90 ml of enrichment media per enrichment bag. An aerobic plate count (APC) showed less than 10 CFU/gram.

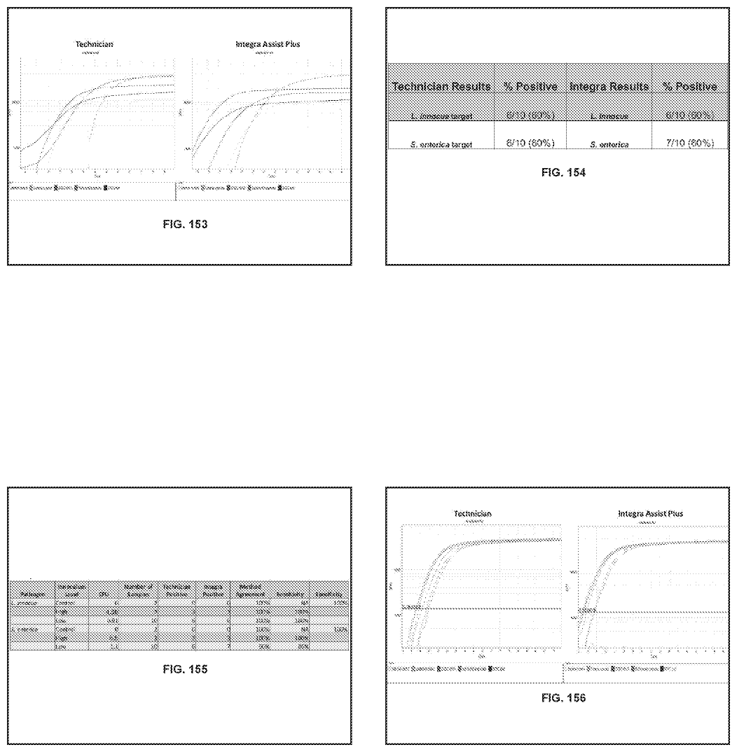

FIG. 154 shows a table of results for Liquid handling robot and Technician Run Samples 1 CFU/sponge on ABI 7500 Fast. FIG. 155 shows a table of results for Liquid handling robot Validation. 90-100% method agreement in detection of all targets at 1 CFU was observed, with sensitivity and specificity meeting AOAC requirements. In conclusion, the technician PCR results for S. enterica with one target showed the method identified 6/10 (60%) of replicates as positive for S. enterica. The results for L. innocula with one target showed the method identified 6/10 (60%) of replicates as positive for L. innocula. In summary, 60-70% detection of all targets at 1 CFU was observed, meeting AOAC requirements.

FIG. 156 shows Liquid handling robot Validation—1 CFU Sponge L. innocua target ABI 7500 Fast. FIG. 157—Liquid handling robot Validation—1 CFU Sponge S. enterica target. FIG. 158—Liquid handling robot Validation—1 CFU Sponge All Targets Present Quantstudio ABI 7500 Fast.

Liquid Handling Robot Validation Study—Environmental Sponges 5 CFU/Sponge

FIG. 159 shows results for Liquid handling robot and Technician Run Samples 5 CFU/sponge on ABI 7500 Fast. FIG. 160 shows a table of results for Liquid handling robot Validation. 100% method agreement in detection of all targets at 5 CFU with sensitivity and specificity meeting AOAC requirements. In conclusion the technician PCR results showed that for S. enterica with one target the method identified 3/3 (100%) of replicates as positive for S. enterica. The results showed that for L. innocua with one target, the method identified 3/3 (100%) of replicates as positive for L. innocua. The liquid handling robot PCR results showed that for S. enterica with one target the method identified 3/3 (100%) of replicates as positive for S. enterica. The results showed that for L. innocua with one target, the method identified 3/3 (100%) of replicates as positive for L. innocua. In summary 100% detection of all pathogens at 5 CFU was observed. FIG. 161—Liquid handling robot Validation 5 CFU Sponge L. innocua target ABI 7500 Fast. FIG. 162 shows Liquid handling robot Validation—5 CFU Sponge S. enterica target ABI 7500 Fast. FIG. 163 shows Liquid handling robot Validation—5 CFU Sponge All Targets Present Quantstudio ABI 7500 Fast.

AOAC Method Comparison Study for Environmental Sponges at 1 CFU/Sponge & 5 CFU/Sponge

A novel Listeria spp. target set were integrated into the existing target sets for Salmonella spp. and STEC E. coli multiplex in place of L. monocytogenes-specific target. The novel target set had high specificity, broad scope, and detect large ranges of Listeria species, including L. monocytogenes, L. innocua, L. grayi, L. welshimeri, L. marthii, L. ivanovii, and L. seeligeri. The target set was shown to work across multiple instrument platforms including QuantStudio 5, and ABI 7500 Fast. A low-level inoculation was performed with 10 replicates of 1 CFU/sponge. Two target organisms were inoculated and incubated simultaneously: 1.10 CFU/sponge of S. enterica (ATCC:13076), and 0.91 CFU/sponge L. innocua (ATCC:33090).

A high level inoculation was performed for 3 replicates of 5 CFU/storage with both organisms inoculated and incubated simultaneously: 5.5 CFU/sponge S. enterica (ATCC:13076), and 4.58 CFU/sponge L. innocua (ATCC:33090). All bacteria were inoculated directly onto the sponge. 18 hour and 24 hour time points were taken for the method and reference methods. 90 ml enrichment medium was used per enrichment bag. The aerobic plate count (APC) was less than 10 CFU/gram.

Samples were enriched at 37° C. for 24 hours in 225 ml of enrichment media. An aerobic plate count (APC) showed less than 10 CFU/gram. The lysis method was performed to maximize recovery and detection of target bacteria DNA. The multiplex qPCR assay was run on the lysate using an ABI 7500 Fast. Up to 288 tests were performed per 96-well PCR plate. Up to 1,150 tests were performed using 384 well format.

In a simultaneous test, FDA BAM and USDA MLG reference methods were used. S. enterica method comparison (USDA MLG 4.08) sponges enriched in 90 ml buffered peptone water (BPW) at 37° C. for 24 hours. After 18 and 24 hours, replicates were streaked on Hektoen chromogenic agar and incubated at 37° C. for 24 hours. Listeria spp. method comparison (USDA MLG 8.1) sponges enriched in 90 ml BLEM at 30° C. for 24 hours. After 18 and 24 hours, replicates were streaked on Modified Oxford Medium (MOX) chromogenic plates and incubated for 24 hours.

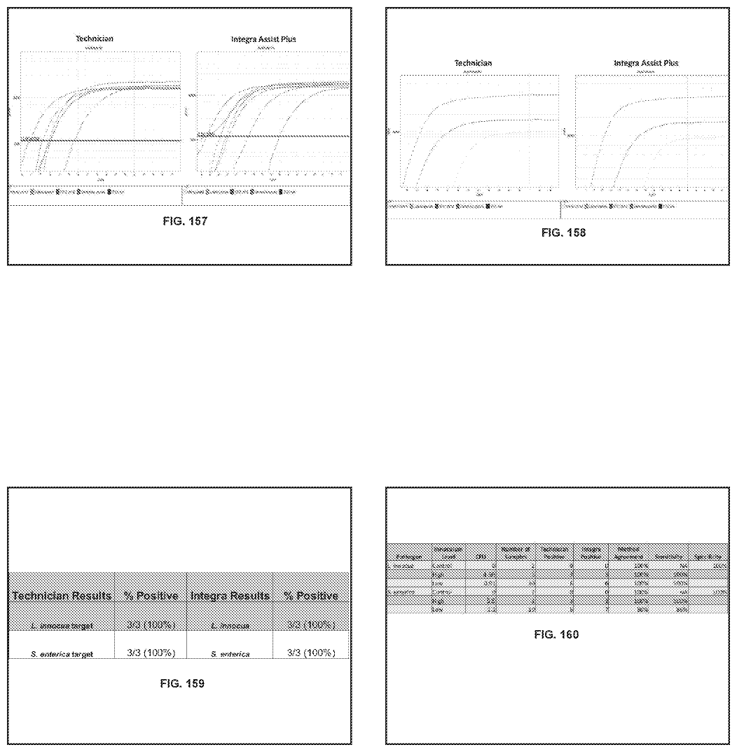

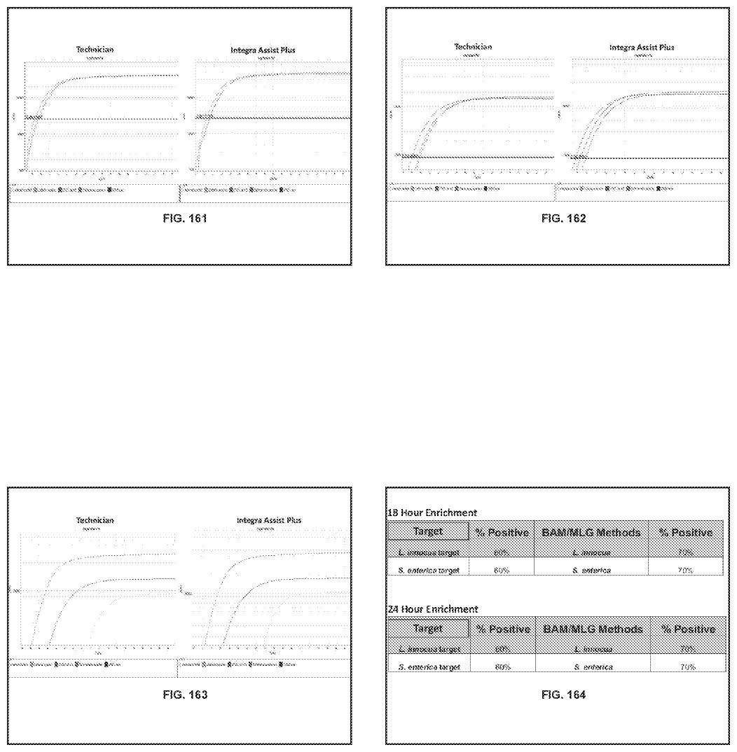

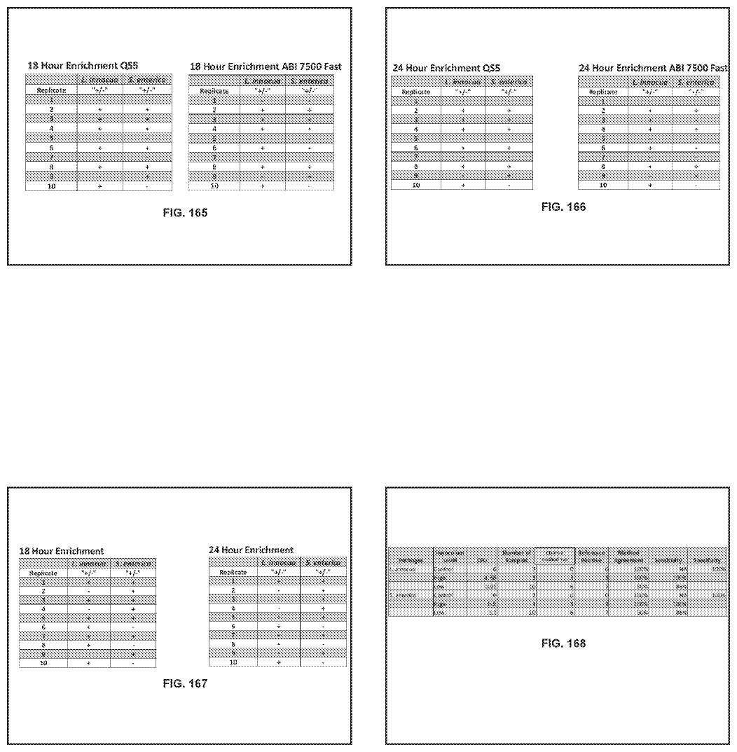

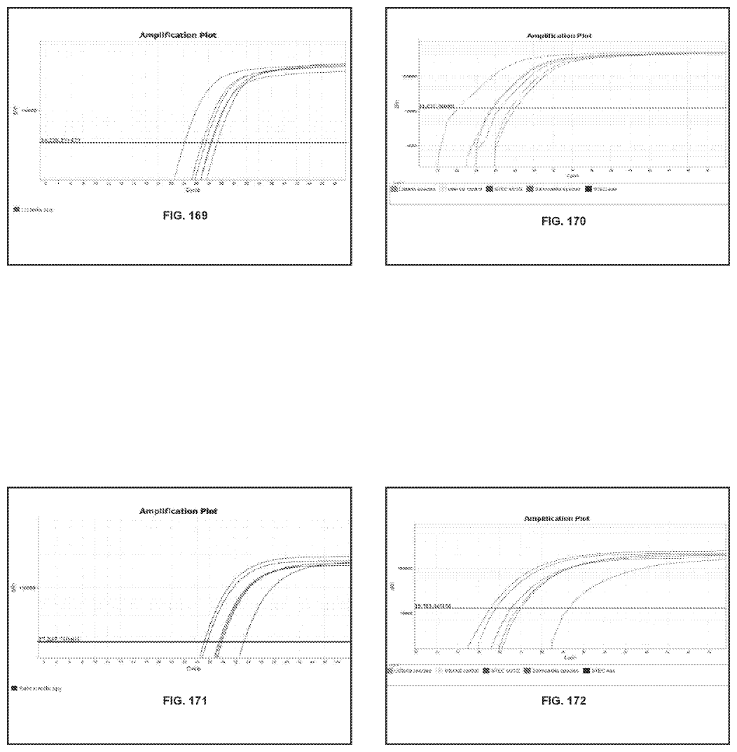

FIG. 164 shows PCR and Method Comparison Results* 1 CFU/sponge. FIG. 165 shows Sponges—1 CFU Quantstudio 5 and ABI 7500 Fast at 18 Hours. 100% agreement at 18 hours was observed for this method on both the QS5 platform and the ABI 7500 Fast platform. FIG. 166—Sponges—1 CFU Quantstudio 5 and ABI 7500 Fast at 24 Hours. 100% agreement at 24 hours was observed for this method on both the QS5 platform and the ABI 7500 Fast platform. FIG. 167—Sponges—1 CFU AOAC BAM/MLG Method Comparison Results at 18 and 24 Hours. FIG. 168 shows a table of results for AOAC Method Comparison. 90-100% method agreement in detection of all targets at 1 CFU was observed, with sensitivity and specificity meeting AOAC requirements.

In conclusion the PCR results showed that for S. enterica with one target, the method identified 6/10 (60%) replicates as positive for S. enterica. For L. innocua with one target, the method identified 6/10 (60%) replicates as positive for L. innocua. For the methods comparison results, S. enterica was identified by USDA MLG 4.08 in 7/10 (70%) of replicates as positive for S. enterica. For L. innocua USDA MLG 8.09 identified 7/10 (70%) of replicates as positive for L. innocua. In summary 60-70% detection of all targets was observed at 1 CFU, meeting AOAC requirements.

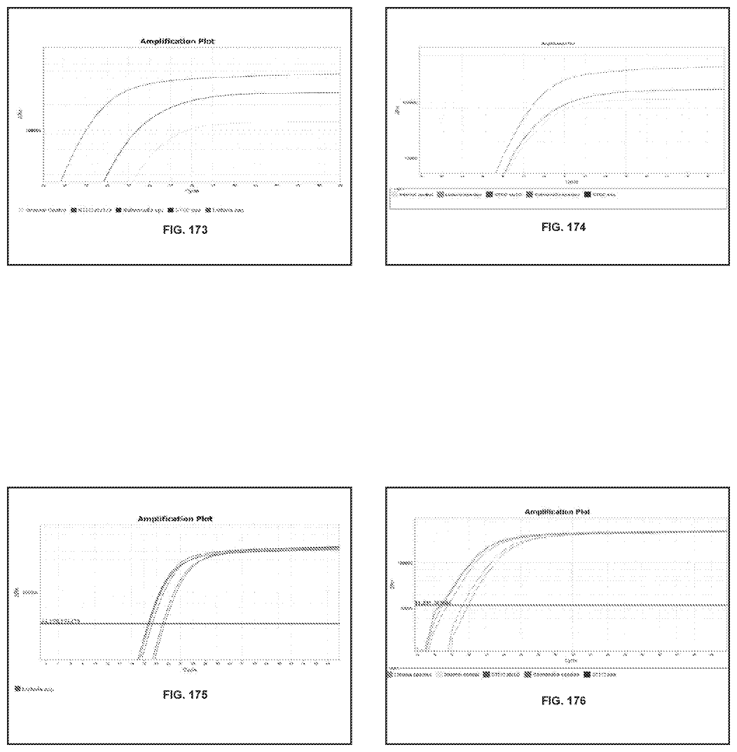

FIG. 169 shows AOAC Method Comparison Validation—1 CFU L. innocua target Quantstudio 5 at 18 Hours. FIG. 170 shows AOAC Method Comparison Validation—1 CFU L. innocua target ABI 7500 Fast at 18 Hours. FIG. 171 shows AOAC Method Comparison Validation—1 CFU S. enterica target Quantstudio 5 at 18 Hours. FIG. 172 shows AOAC Method Comparison Validation—1 CFU S. enterica target ABI 7500 Fast at 18 Hours. FIG. 173 shows AOAC Method Comparison Validation—1 CFU Both Targets present on Quantstudio 5 at 18 Hours. FIG. 174 AOAC Method Comparison Validation—1 CFU Both Targets present on ABI 7500 Fast at 18 Hours. FIG. 175 shows AOAC Method Comparison Validation—1 CFU

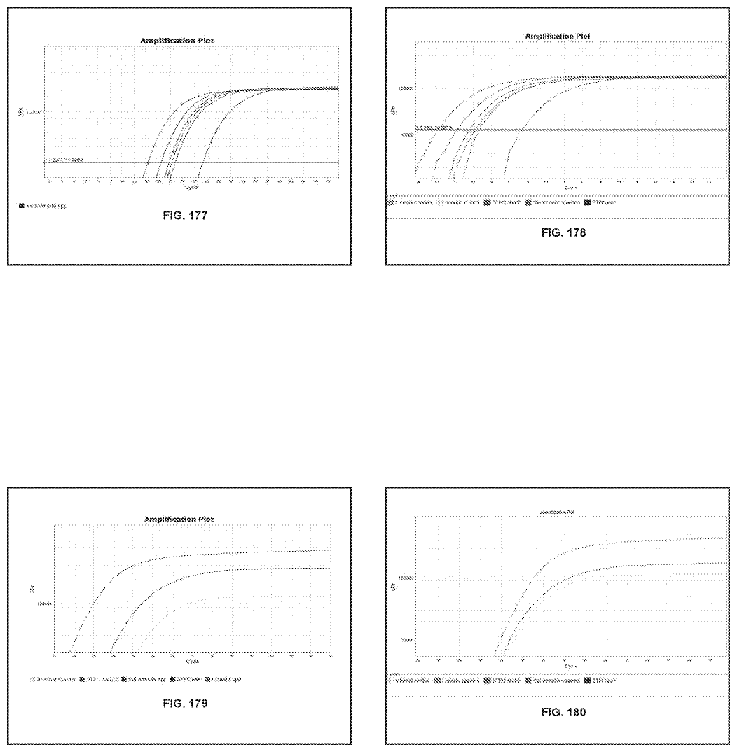

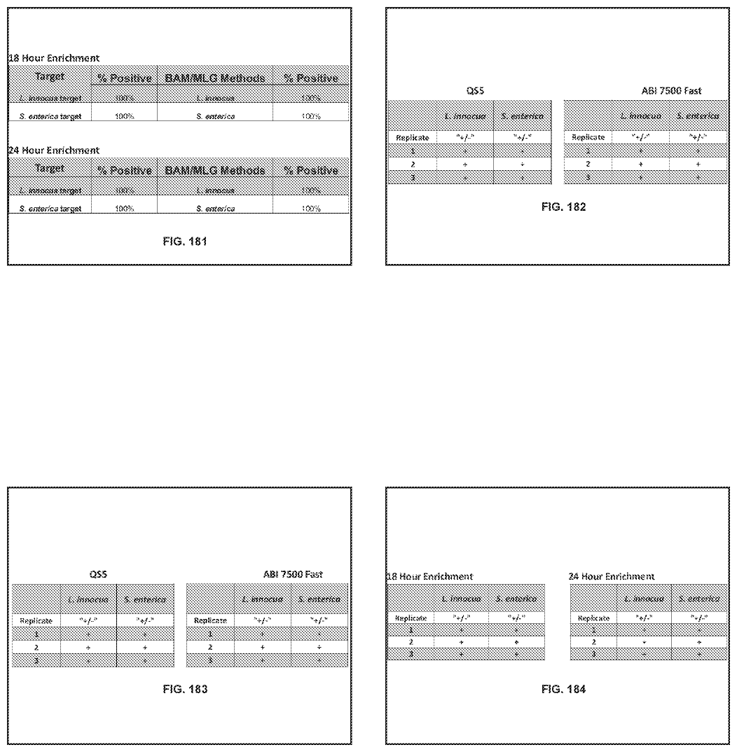

L. innocua target Quantstudio 5 at 24 Hours. FIG. 176 shows AOAC Method Comparison Validation—1 CFU L. innocua target ABI 7500 Fast at 24 Hours. FIG. 177 shows AOAC Method Comparison Validation—1 CFU S. enterica target Quantstudio 5 at 24 Hours. FIG. 178 shows AOAC Method Comparison Validation—1 CFU S. enterica target ABI 7500 Fast at 24 Hours. FIG. 179 shows AOAC Method Comparison Validation—1 CFU Both Targets present on Quantstudio 5 at 24 Hours. FIG. 180 shows AOAC Method Comparison Validation—1 CFU Both Targets present on ABI 7500 Fast at 24 Hours.

AOAC Method Comparison Study—Environmental Sponges—5 CFU/Sponge

A high level inoculation was performed for 3 replicates of 5 CFU/storage with both organisms inoculated and incubated simultaneously: 5.5 CFU/sponge S. enterica (ATCC:13076), and 4.58 CFU/sponge L. innocua (ATCC:33090). All bacteria were inoculated directly onto the sponge. 18 hour and 24 hour time points were taken for the method and reference methods. 90 ml enrichment medium was used per enrichment bag. The aerobic plate count (APC) was less than 10 CFU/gram.

Samples were enriched at 37° C. for 18 and 24 hours in 225 ml of enrichment media. An aerobic plate count (APC) showed less than 10 CFU/gram. The lysis method was performed to maximize recovery and detection of target bacteria DNA. The multiplex qPCR assay was run on the lysate using an ABI 7500 Fast. Up to 288 tests were performed per 96-well PCR plate. Up to 1,150 tests were performed using 384 well format.

FIG. 181 shows PCR and Method Comparison Results* 5 CFU/sponge. As this was an unpaired study the positive replicates were not numerically matched when comparing the PCR data to the BAMIMLG methods. FIG. 182 Environmental Sponge 5 CFU—QuantStudio 5 and ABI 7500 Fast Results at 18 Hours. The method detected all targets in 100% of replicates at a 5 CFU inoculation level on both platforms. FIG. 183 Environmental Sponge 5 CFU—QuantStudio 5 and ABI 7500 Fast Results at 24 Hours. The method detected all targets in 100% of replicates at a 5 CFU inoculation level on both platforms. FIG. 184 shows Sponges—5 CFU AOAC BAM/MLG Method Comparison Results at 18 and 24 Hours. FIG. 185 shows a table of results for AOAC Method Comparison. Conclusion. 100% method agreement in detection of all targets at 5 CFU at 18 and 24 hours was observed, with sensitivity and specificity meting AOAC requirements. In conclusion for the environmental sponge at 5 CFU, with S. enterica the method identified 3/3 (100%) of replicates as positive for S. enterica. For L. innocua with one target the method identified 3/3 (100%) replicates as positive for L. innocua. For the methods comparison results the S. enterica with USDA MLG 4.08 identified 3/3 (100%) of replicates as positive for S. enterica. For L. innocua USDA MLG 8.09 identified 3/3 (100%) of replicates as positive for L. innocua. In summary 100% detection of all pathogens was observed at 5 CFU.

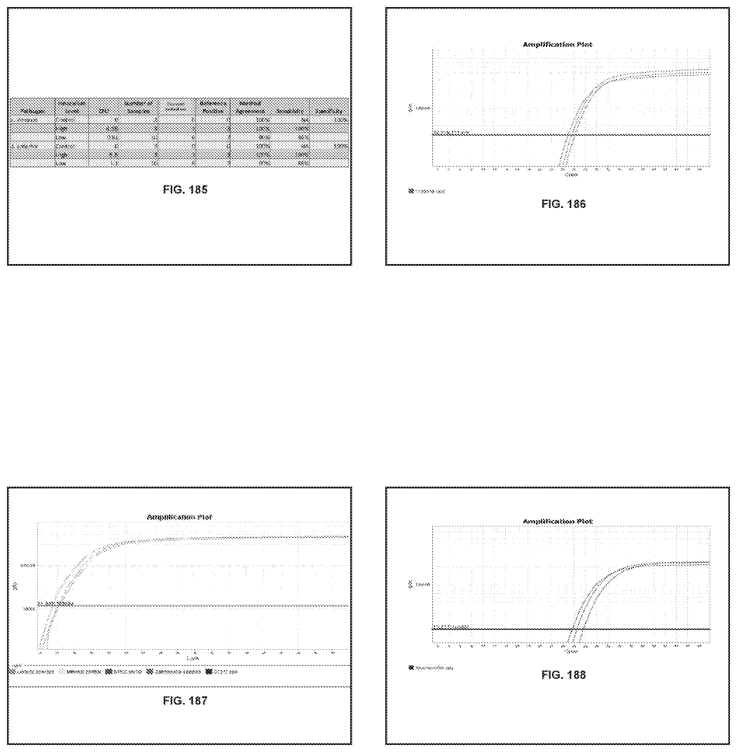

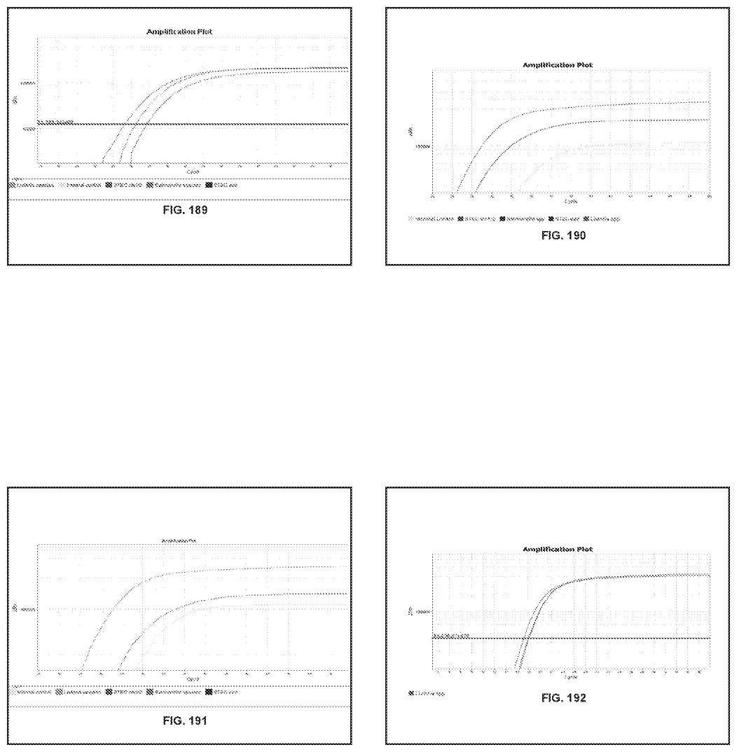

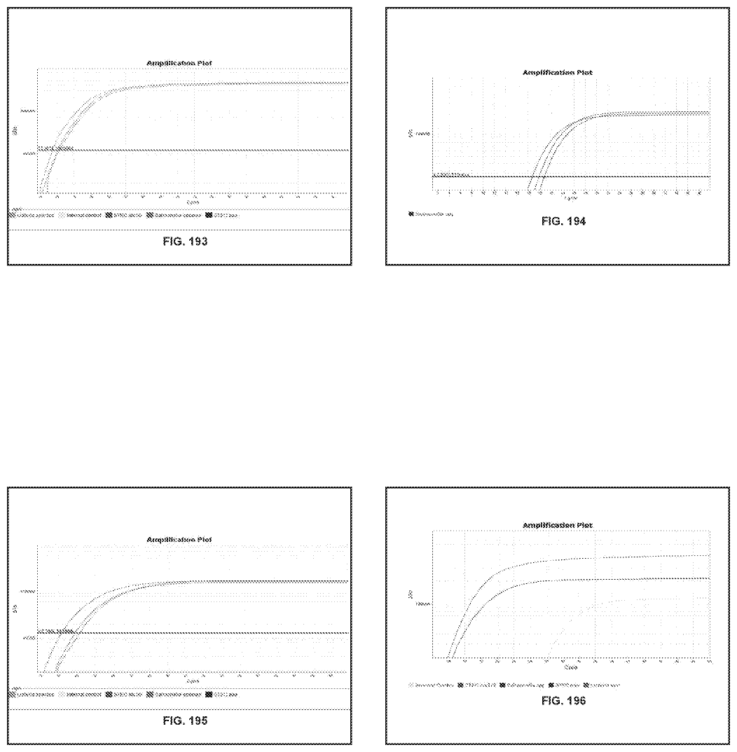

FIG. 186 shows AOAC Method Comparison Validation—5 CFU L. innocua target Quantstudio 5 at 18 Hours. The method detected L. innocua in 3/3 replicates (100% recovery). FIG. 187 shows AOAC Method Comparison Validation—5 CFU L. innocua target ABI 7500 Fast at 18 Hours. The method detected L. innocua in 3/3 replicates (100% recovery). FIG. 188 shows AOAC Method Comparison Validation—5 CFU S. enterica target Quantstudio 5 at 18 Hours. The method detected S. enterica in 3/3 replicates (100% recovery). FIG. 189 shows AOAC Method Comparison Validation—5 CFU S. enterica target ABI 7500 Fast at 18 Hours. The method detected S. enterica in 3/3 replicates (100% recovery). FIG. 190 shows AOAC Method Comparison Validation—5 CFU Both Targets present on Quantstudio 5 at 18 Hours. The method detected both pathogens in the same replicate. FIG. 191 shows AOAC Method Comparison Validation—5 CFU Both Targets present on ABI 7500 Fast at 18 Hours. The method detected both pathogens in the same replicate. FIG. 192 shows AOAC Method Comparison Validation—5 CFU L. innocua target Quantstudio 5 at 24 Hours. The method detected S. enterica in 3/3 replicates (100%) recovery. FIG. 193 shows AOAC Method Comparison Validation—5 CFU L. innocua target ABI 7500 Fast at 24 Hours. The method detected S. enterica in 3/3 replicates (100%) recovery. FIG. 194 shows AOAC Method Comparison Validation—5 CFU S. enterica target Quantstudio 5 at 24 Hours. The method detected S. enterica in 3/3 replicates (100%) recovery. FIG. 195 shows AOAC Method Comparison Validation—5 CFU S. enterica target ABI 7500 Fast at 24 Hours. The method detected S. enterica in 3/3 replicates (100%) recovery. FIG. 196 shows AOAC Method Comparison Validation—5 CFU Both Targets present on Quantstudio 5 at 24 Hours. The method detected both targets in the same replicate. FIG. 197 shows AOAC Method Comparison Validation—5 CFU Both Targets present on ABI 7500 Fast at 24 Hours. The method detected both targets in the same replicate.

Validation Data for Hemp—2 CFU/1 g & 15 CFU/1 g—ABI QuantStudio5

All bacteria were inoculated into CBD-containing hemp strain (Suver Haze, Tweedle Farms) and incubated at 37° C. This was also used as a surrogate for Cannabis leaves. Two target organisms were inoculated and enriched simultaneously: E. coli)157:H7 (ATCC:43859), and S. enterica (ATCC:13076). A low-level inoculation was performed with 15 replicates of 2 CFU/1 g per matrix. 2 CFU is within 0.2-2.0 CFU range of lower limit of detection. These were inoculated into 1 g matrix. The amounts used were 2.65 CFU/25 g E. coli, and 3.3 CFU/25 g S. enterica. A high-level inoculation showed 15 replicates of 15 CFU/1 g per matrix. 15 CFU inoculation was used to demonstrate efficacy in high-titer levels of pathogens. These were inoculated into 1 g of matrix. The amounts used were 19.8 CFU/25 g E. coli, and 24.7 CFU/25 g S. enterica.

Samples were enriched at 37° C. for 24 hours in 225 ml of enrichment media. An aerobic plate count (APC) showed less than 10 CFU/gram. The lysis method was performed to maximize recovery and detection of target bacteria DNA. The multiplex qPCR assay was run on the lysate using an ABI QuantStudio (96-well format).

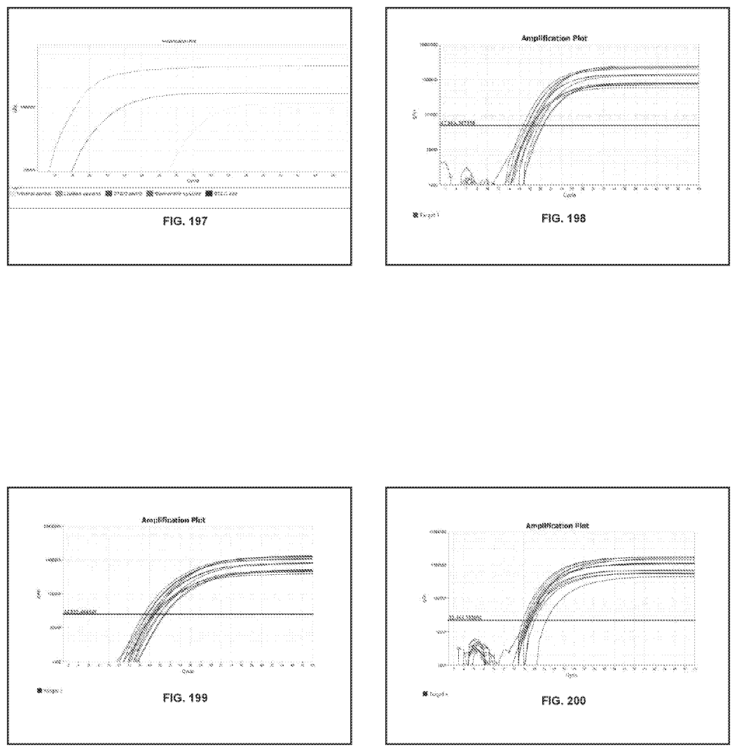

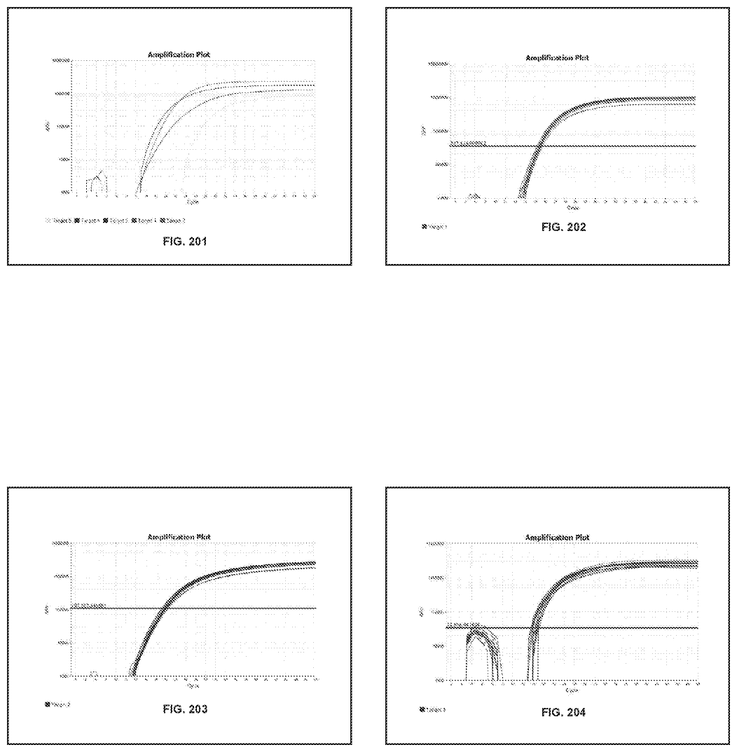

FIG. 198 shows QuantStudio5, Hemp—2 CFU E. coli O157:H7 STEC STX-1 and STX-2. The method detected STX-1 and STX-2 targets in 14/15 replicates (93% recovery). FIG. 199 shows QuantStudio5, Hemp—2 CFU E. coli O157:H7 STEC EAE target. The method detected E. Coli EAE in 14/15 replicates (93% recovery). FIG. 200 shows ABI QuantStudio5 Hemp—2 CFU S. enterica target. The method detected the S. enterica target in 14/15 replicates (93% recovery). FIG. 201 shows QuantStudio5 All Targets in a Single Reaction—2 CFU. This figure is an example of the method detecting all pathogen targets in a single enrichment and PCR reaction. The results for the Shiga Toxin E. coli (STEC) targets show that for STX-1/STX-2 the method identified 93% of replicates as positive for STX-1/STX-2. For EAE the method identified 93% of replicates as positive for EAE. For Salmonella enterica the method identified 93% of replicates as positive for S. enterica. The internal control was detected on all replicates. In summary 93% detection of all targets at 2 CFU/1 g was observed. FIG. 202 shows QuantStudio5, Hemp—15 CFU E. coli O157:H7 STEC STX-1 and STX-2. The method detected STX-1 and STX-2 in 15/15 replicates (100% recovery). FIG. 203 shows QuantStudio5, Hemp—15 CFU E. coli O157:H7 STEC EAE target. The method detected E. coli EAE in 5/5 replicates (100% recovery). FIG. 204 shows QuantStudio5, Hemp—15 CFU S. enterica target. The method detected S. enterica in 15/15 replicates (100% recovery). FIG. 205 shows QuantStudio5, Hemp 15 CFU All Targets Present. This figure shows that the method can detect all targets when present in the same reaction. In conclusion, for the Shiga Toxin E. coli (STEC) targets the method identified 100% of replicates as positive for STX-1/STX-2. For the EAE the method identified 100% replicates as positive for EAE. For Salmonella enterica the method identified 100% of replicates as positive for S. enterica. The internal control was detected on all replicates. In summary 100% detection of all targets at 15 CFU/1 g was observed.

Liquid Handling Robot Validation—RTE Pork Sausage and Environmental Sponges

A liquid handling robot was used to prepare reactions. A novel Listeria spp. target set was used with Salmonella spp. and STEC E. coli multiplex in place of a L. monocytogenes-specific target. The Listeria spp. target set showed broad scope, detecting a large range of Listeria species including L. monocytogenes, L. innocua, L. grayi, L. welshimeri, L. marthii, L. ivanovii, and L. seeligeri. The target set was shown to work across multiple instrument platforms including QuantStudio 5, and ABI 7500 Fast. Liquid robot handling samples were directly compared to the same set of samples run by laboratory personnel. Both sets of samples were run on same 96-well PCR plate.

A low level inoculation was performed for 20 replicates at 1 CFU/25 g of cooked pork sausage. 1 CFU inoculation is the AOAC-required lower limit of detection. All three target organisms were inoculated and incubated simultaneously. 1.37 CFU/25 g of E. coli O157:H7 (ATC:43895), 1.13 CFU/25 g for S. enterica (ATCC:13076), and 1.5 CFU/25 g L. innocua (ATCC:33090). A high level inoculation was performed with 5 replicates of 5 CFU/25 g of pork sausage. All three target organisms were inoculated and incubated simultaneously. 6.85 CFU/25 g E. coli O157:H7 (ATCC:43895), 5.65 CFU/25 g S. enterica (ATCC:13076), 7.5 CFU/25 g L. innocua (ATCC:33090). All bacteria were inoculated into pork sausage and stored at 4° C. for 48 hours, as per AOAC guidelines. An aerobic plate count (APC) showed less than 10 CFU/gram. Samples were enriched at 37° C. for 24 hours in 225 ml of enrichment media. The lysis method was performed to maximize recovery and detection of target bacteria DNA. The multiplex qPCR assay was run on an ABI 7500 Fast. Up to 288 tests were performed per 96-well PCR plate. Up to 1,150 tests were performed using 384 well format.

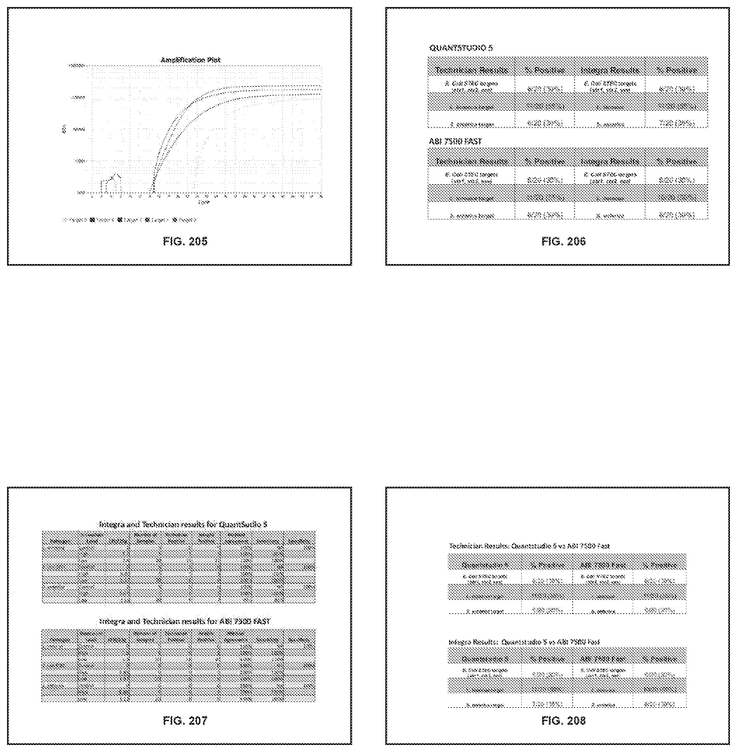

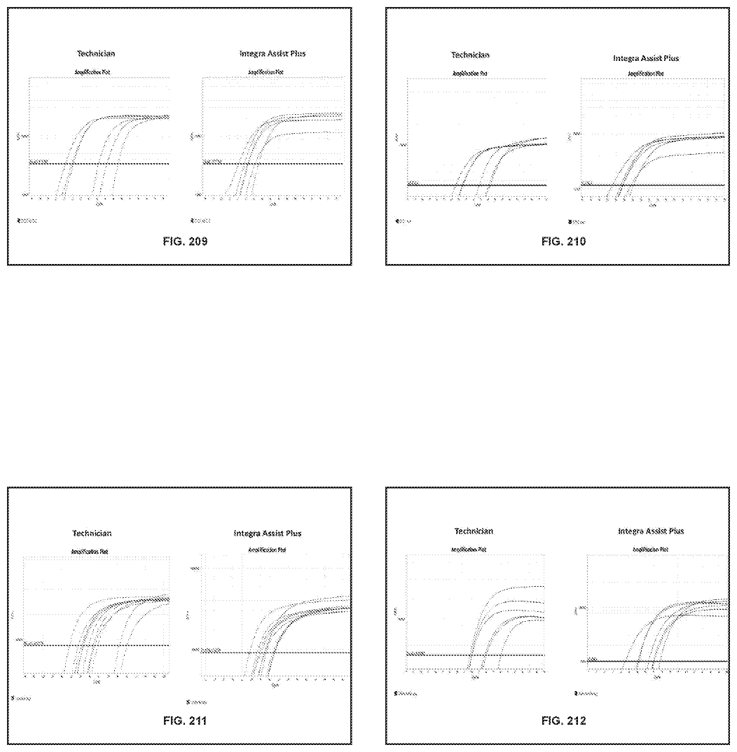

FIG. 206 shows Results for Liquid handling robot and Technician Run Samples 1 CFU/25 g Pork Sausage on QuantStudio 5 and ABI 7500 Fast. FIG. 207 shows a table of results for Liquid handling robot Validation. FIG. 208 shows Results for Liquid handling robot and Technician Run Samples1 CFU/25 g Pork Sausage on QuantStudio 5 and ABI 7500 Fast.

In conclusion the technician generated results showed that for E. coli (STEC) targets, STX-1/STX-2 the method identified 6/20 (30%) of replicates as positive for STX-1/STX-2, for EAE the method identified 6/20 (30%) of replicates as positive for EAE. For S. enterica with one target the method identified 6/20 (30%) replicates as positive for S. enterica. For L. innocua with one target the method identified 11/20 (55%) replicates as positive for L. innocua. The liquid handling robot generated results showed that for E. coli (STEC) targets, STX-1/STX-2 the method identified 6/20 (30%) of replicates as positive for STX-1/STX-2, for EAE the method identified 6/20 (30%) of replicates as positive for EAE. For S. enterica with one target the method identified 7/20 (35%) replicates as positive for S. enterica. For L. innocua with one target the method identified 11/20 (55%) replicates as positive for L. innocua. In summary, 30-55% detection of all targets at 1 CFU/25 g was observed, meeting AOAC fractional recovery requirements.

FIG. 209 shows Liquid handling robot Validation—1 CFU Pork Sausage E. coli O157:H7 Quantstudio 5. The liquid handling robot and technician-run samples both detected the stx1 and stx2 targets in 6/20 replicates (30% recovery). FIG. 210 shows Liquid handling robot Validation—1 CFU Pork Sausage E. coli O157:H7 Quantstudio 5. The liquid handling robot and technician-run samples both detected the eae target in 6/20 replicates (30% recovery). FIG. 211 shows Liquid handling robot Validation—1 CFU Pork Sausage L. innocua target Quantstudio 5. The liquid handling robot and technician run samples both detected the Listeria spp. target in 11/20 replicates (55% recovery). FIG. 212 shows Liquid handling robot Validation—1 CFU Pork Sausage S. enterica target Quantstudio 5. The technician-run samples detected the S. enterica target in 6/20 replicates (30% recovery). The liquid handling robot samples detected the S. enterica target in 7/20 replicates (35% recovery). FIG. 213 shows Liquid handling robot Validation—1 CFU Pork Sausage All Targets Present Quantstudio 5 All targets present in a single reaction.

Liquid Handling Robot Validation Study with QuantStudio 5—Pork Sausage—5 CFU/25 g

All target organisms were inoculated and enriched simultaneously; E. coli O157:H7 (ATCC:43895), S. enterica (ATCC:13076) and L. innocua (ATCC:33090). All bacteria were inoculated into pork sausage and stored at 4° C. for 48 hours, as per AOAC guidelines. Aerobic plate counts (APC) had less than 10 CFU/gram. Samples were enriched at 37° C. for 24 hours in 22 ml of PMEM. A lysis procedure as previously described herein was performed to maximize recovery and detection of target bacteria DNA. Amplification and detection of lysate was performed on a multiplex qPCR assay run on a ThermoFisher™ Quantstudio 5 instrument. Up to 288 tests were run on each 96-well PCR plate. 1,150 tests were run using 384 well format.

A high-level inoculation was performed of 5 replicates of 5 CFU/25 g of pork sausage: 3.42 CFU/25 g E. coli O157:H7 (ATCC:43895), 4.30 CFU/25 g S. enterica (ATCC:13076), 5.5 CFU/25 g L. innocua (ATCC:33090).

Samples were enriched at 37° C. for 24 hours in the selective enrichment media. Samples were lysed using the lysis procedure to maximize recovery and detection of bacterial DNA. The method was performed on the lysate using real time PCR detection of nucleic acid targets on a ThermoFisher™ ABI 7500 Fast (96-well format) machine. Primers and probes used are disclosed in Table 1.

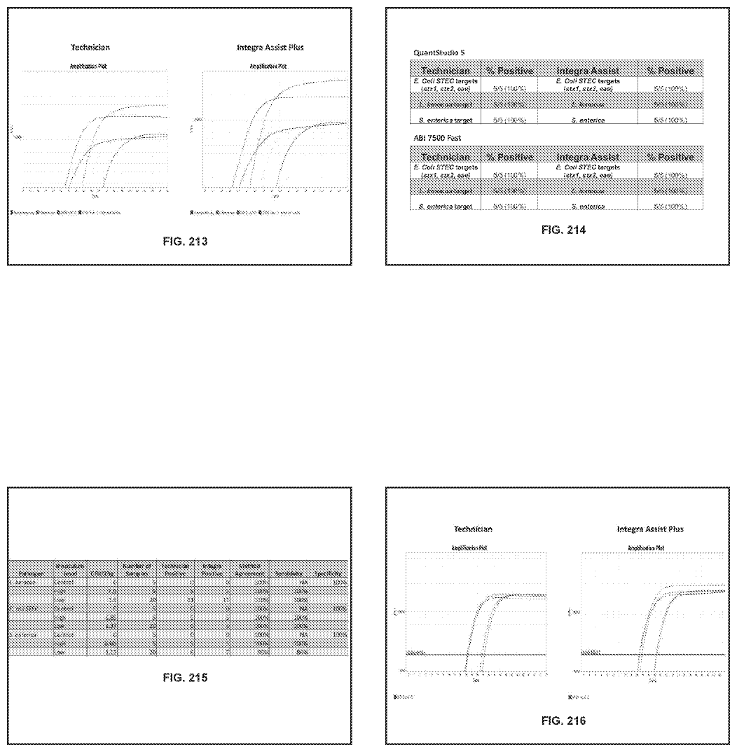

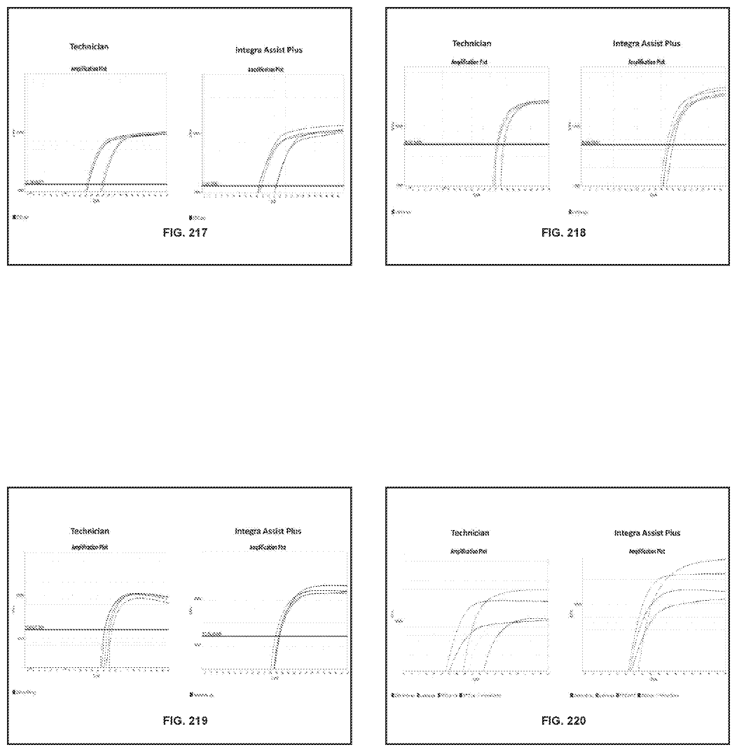

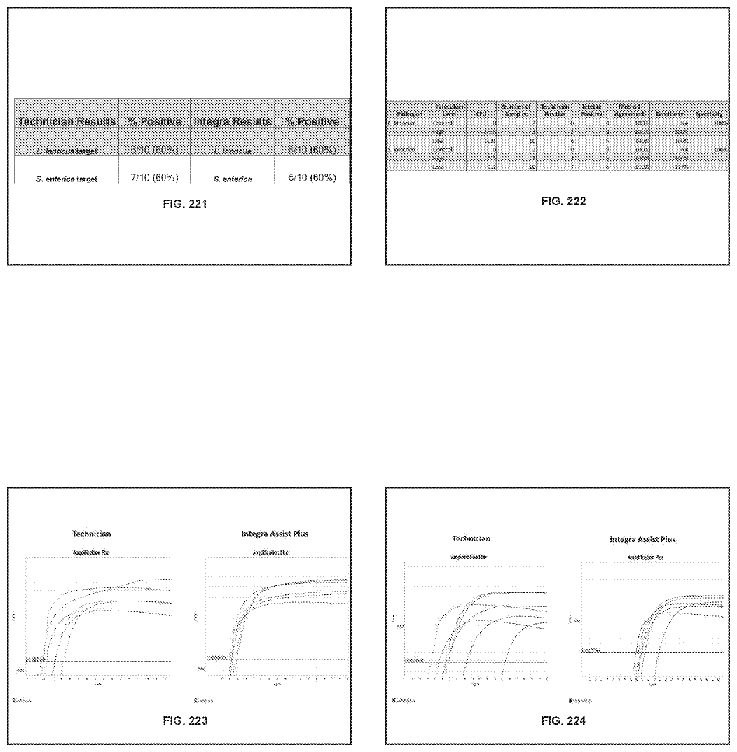

FIG. 214 shows Results for Liquid handling robot and Technician Run Samples 5 CFU/25 g Pork Sausage. FIG. 215 shows Liquid handling robot Validation table of Results. 100% method agreement in detection of all targets at 5 CFU was observed, with sensitivity and specificity meeting AOAC requirements. FIG. 216 shows Liquid handling robot Validation—5 CFU Pork Sausage E. coli O157:H7 Quantstudio 5. The liquid handling robot and technician-run samples both detected the stx1 and sbc2 targets in 5/5 replicates (100% recovery). FIG. 217 shows Liquid handling robot Validation—5 CFU Pork Sausage E. coli O157:H7 Quantstudio 5. The integra and technician-run samples both detected the eae target in 5/5 replicates (100% recovery). FIG. 218 shows Liquid handling robot Validation—5 CFU Pork Sausage L. innocua target Quantstudio 5. The integra and technician-run samples both detected the Listeria spp. target in 5/5 replicates (100% recovery). FIG. 219 shows Liquid handling robot Validation—5 CFU Pork Sausage S. enterica target Quantstudio 5. The integra and technician-run samples both detected the S. enterica target in 5/5 replicates (100% recovery). FIG. 220 shows Liquid handling robot Validation—5 CFU Pork Sausage All Targets Present Quantstudio 5. All targets were present and detected in a single reaction.

Liquid Handling Robot Validation—Environmental Sponges

Two target organisms were inoculated and enriched simultaneously; S. enterica (ATCC:13076) and L. innocua (ATCC:33090). All bacteria were inoculated directly onto environmental sponges. Samples were enriched in 90 ml of enrichment medium per enrichment bag. Aerobic plate counts (APC) had less than 10 CFU/gram. A lysis procedure as previously described herein was performed to maximize recovery and detection of target bacteria DNA. Amplification and detection of lysate was performed on a multiplex qPCR assay run on a ThermoFisher™ Quantstudio 5 instrument. Up to 288 tests were run on each 96-well PCR plate. 1,150 tests were run using 384 well format.

FIG. 221 shows Results for Liquid handling robot and Technician Run Samples 1 CFU/sponge on QuantStudio 5. FIG. 222 shows a table of results for Liquid handling robot Validation. 100% method agreement in detection of all targets at 1 CFU was observed, with sensitivity and specificity meeting AOAC requirements. In conclusion the technician results showed that for S. enterica with one target, the method identified 7/10 (70%) of replicates as positive for S. enterica. For L. innocua the method identified 6/10 (60%) replicates as positive for L. innocua. For the liquid handling robot PCR results, for S. enterica with one target, the method identified 6/10 (60%) of replicates as positive for S. enterica. For L. innocua the method identified 6/10 (60%) replicates as positive for L. innocua. In summary, 60-70% detection of all targets at 1 CFU was observed, meeting AOAC requirements.

FIG. 223 shows Liquid handling robot Validation—1 CFU Sponge L. innocua target Quantstudio 5. The liquid handling robot and the technician run samples both detected the Listeria spp. target in 6/10 replicates (60% recovery). FIG. 224 shows Liquid handling robot Validation—1 CFU Sponge S. enterica target. The technician-run samples detected the S. enterica target in 7/10 replicates (70% recover). The liquid handling robot samples detected the S. enterica target in 6/10 replicates (60% recovery). FIG. 225 shows Liquid handling robot Validation—1 CFU Sponge All Targets Present Quantstudio 5. All targets were present in a single reaction.

FIG. 226 shows Results for Liquid handling robot and Technician Run Samples 5 CFU/sponge on QuantStudio 5. FIG. 227 shows a table of results for Liquid handling robot Validation. 100% method agreement in detection of all targets at 5 CFU was observed, with sensitivity and specificity meeting AOAC requirements.

In conclusion the technician generated PCR results showed that for S. enterica with one target, the method identified 3/3 (100%) of replicates as positive for S. enterica. For L. innocular with one target, the method identified 3/3 (100%) of replicates as positive for L. innocua. The liquid handling robot results showed that for S. enterica with one target, the method identified 3/3 (100%) of replicates as positive for S. enterica. For L. innocular with one target, the method identified 3/3 (100%) of replicates as positive for L. innocua. In summary, 100% detection of all pathogens at 5 CFU was observed.

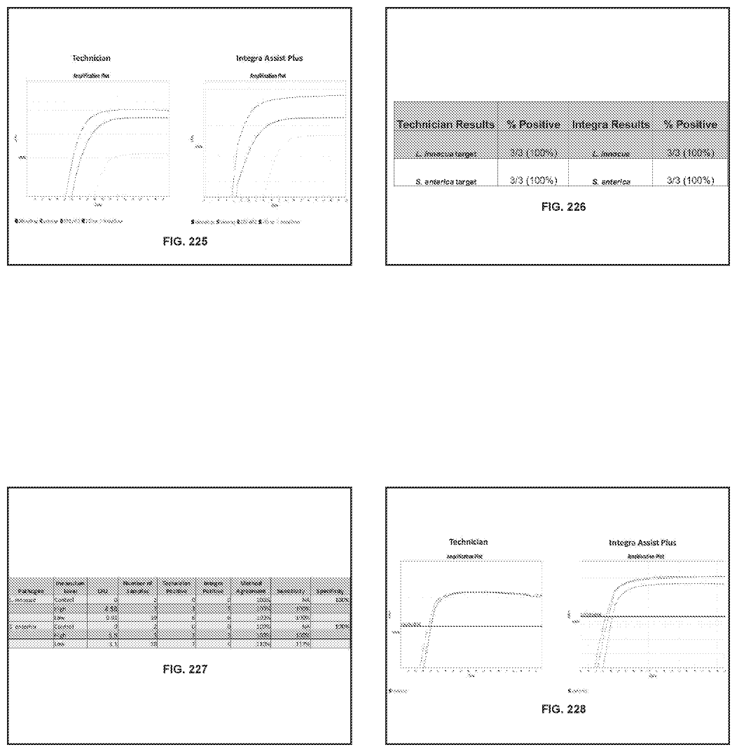

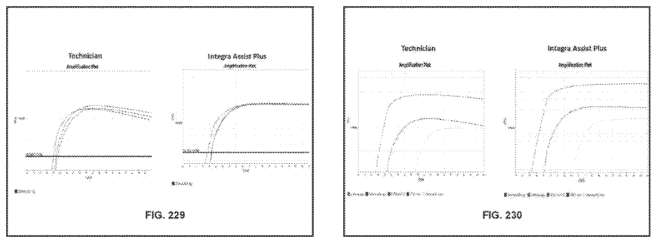

FIG. 228 shows Liquid handling robot Validation—5 CFU Sponge L. innocua target Quantstudio 5. The liquid handling robot and technician-run samples both detected the Listeria spp. target in 3/3 replicates (100% recovery). FIG. 229 shows Liquid handling robot Validation—5 CFU Sponge S. enterica target Quantstudio 5. The liquid handling robot and technician-run samples bot detected the S. enterica target in 3/3 replicates (100% recovery). FIG. 230 shows Liquid handling robot Validation—5 CFU Sponge All Targets Present Quantstudio 5. All targets are present and detected in a single reaction.

Primers and Buffers

Disclosed herein are primers, oligonucleotides probes, methods, materials, compositions, kits, and components that can be used for, can be used in conjunction with, can be used in preparation for, or are products of methods and compositions disclosed herein. It is understood that when combinations, subsets, interactions, groups, etc. of these materials are disclosed and while specific reference of each various individual and collective combinations and permutation of these molecules and compounds cannot be explicitly disclosed, each is specifically contemplated and described herein. For example, if a nucleotide or nucleic acid is disclosed and discussed and a number of modifications that can be made to a number of molecules including the nucleotide or nucleic acid are discussed, each and every combination and permutation of nucleotide or nucleic acid and the modifications that are possible are specifically contemplated unless specifically indicated to the contrary. This concept applies to all aspects of this application including, but not limited to, steps in methods of making and using the disclosed methods and compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that in some embodiments, the kits disclosed herein may comprise instructions to combine and or use the contents of said kits. In some embodiments, the instructions may comprise instructions of how to combine and or use a lysis buffer. In some embodiments, the instructions may comprise instructions of how to combine and or use a buffering component. In some embodiments, the instructions may comprise instructions of how to combine and or use a metal chelating agent. In some embodiments, the instructions may comprise instructions of how to combine and or use a surfactant. In some embodiments, the instructions may comprise instructions of how to combine and or use a precipitant. In some embodiments, the instructions may comprise instructions of how to combine and or use lysing moieties. In some embodiments, the instructions may comprise instructions of how to combine and or use amplification primers. In some embodiments, the instructions may comprise instructions of how to combine and or use a lysis buffer and amplification primers. In some embodiments, the instructions may comprise instructions of how to combine and or use an internal oligonucleotide probe. In some embodiments, the instructions may comprise instructions of how to combine and or use a lysis buffer, amplification primers and an internal oligonucleotide probe.

While some embodiments described herein have been shown and described herein, such embodiments are provided by way of example only. Numerous variations, changes, and substitutions will now occur to those skilled in the art without departing from the disclosure provided herein. It should be understood that various alternatives to the embodiments described herein can be employed in practicing the methods described herein.

1-50. (canceled)

51. A method of detecting the presence or absence of two or more pathogens in a sample, the method comprising:
(a) performing an amplification of a selective enrichment media contacted sample; and
(b) detecting the presence or absence of the two or more pathogens, wherein the two or more pathogens comprises:
(i) at least one pathogen from
(1) Escherichia,
(2) Salmonella; and
(ii) at least one pathogen from:
(1) Listeria species, wherein the Listeria species is selected from the group consisting of L. aquatica, L. booriae, L. cornellensis, L. costaricensis, L. goaensis, L. fleischmannii, L. floridensis, L. grandensis, L. grayi, L. innocua, L. ivanovii, L. marthii, L. newyorkensis, L. riparia, L. rocourtiae, L. seeligeri, L. thailandensis, L. weihenstephanensis, and L. welshimeri.

52. The method of claim 51, wherein the amplification is performed with a set of amplification primers.

53. The method of claim 51, wherein the method is performed within a positive total time of about 28 hrs.

54. The method of claim 51, wherein the sample is suspended in the selective enrichment media such that the two or more pathogens are isolated from the sample,
wherein the two or more pathogens are isolated from the sample by stomaching.

55. The method of claim 54, wherein the sample is stomached for at least about 30 seconds.

56. The method of claim 51, wherein the sample is enriched at a temperature in the range of about 30° C. to about 45° C.

57. The method of claim 54, wherein the sample is incubated for a positive amount of time less than or equal to about 24 hours following stomaching.

58. The method of claim 51, wherein the sample is lysed by incubating the sample with a lysis buffer, wherein the lysis buffer comprises:
(a) a buffering component;
(b) a metal chelating agent;
(c) a surfactant;
(d) a precipitant; and
(e) at least two lysing moieties.

59. The method of claim 58, wherein the buffering component comprises tris (hydroxymethyl) aminomethane (TRIS), wherein tris (hydroxymethyl) aminomethane (TRIS) is present at a concentration in the range of about 60 mM to about 100 mM.

60. The method of claim 58, wherein the metal chelating agent comprises ethylenediamine-tetraacetic acid (EDTA), and wherein ethylenediaminetetraacetic acid (EDTA) is present at a concentration in the range of about 1 mM to about 18 mM.

61. The method of claim 58, wherein the surfactant comprises polyethylene glycol p-(1, 1, 3, 3-tetramethylbutyl)-phenyl ether (Triton-X-100), and wherein the polyethylene glycol p-(1, 1, 3, 3-tetramethylbutyl)-phenyl ether (Triton-X-100) is present at a concentration in the range of about 0.1% to about 10%.

62. The method of claim 58, wherein the precipitant comprises proteinase K, and wherein proteinase K is present at a concentration in the range of about 17.5% to about 37.5%.

63. The method of claim 58, wherein the at least two lysing moieties comprises a lysis bead, wherein the lysis bead comprises 100 μm zirconium lysis beads, and wherein the 100 μm zirconium lysis beads are present at a concentration in the range of about 0.1 grams/ml to about 2.88 grams/ml.

64. The method of claim 58, wherein the lysing moiety comprises a lysozyme, wherein the lysozyme is present at a concentration in the range of about 10 mg/ml to about 30 mg/ml.

65. The method of claim 51, wherein the detecting is reported by a communication medium.

66. The method of claim 51, wherein the sample comprises cannabis.

67. The method of claim 51, wherein the sample comprises Hemp.

68. The method of claim 51, wherein the sample comprises CBD oil.

69. The method of claim 51, wherein the method is performed without extracting nucleic acids from the two or more pathogens, and wherein the nucleic acids comprise DNA, RNA or a combination thereof.

70. The method of claim 51, wherein the two or more pathogens comprise Escherichia, Salmonella and Listeria species.
